SEQUENCE LISTING (66 sequences)

SEQ ID NO: 1 - protein, 496 aa, Escherichia coli K-12 MG1655
MSTTTQNIPWYRHLNRAQWRAFSAAWLGYLLDGFDFVLIALVLTEVQGEFGLTTVQAASLISAAFISRWFGGLMLGAMGDRYGRRLAMVTSIVLFSAGTLACGFAPGYITMFIARLVIGMGMAGEYGSSATYVIESWPKHLRNKASGFLISGFSVGAVVAAQVYSLVVPVWGWRALFFIGILPIIFALWLRKNIPEAEDWKEKHAGKAPVRTMVDILYRGEHRIANIVMTLAAATALWFCFAGNLQNAAIVAVLGLLCAAIFISFMVQSAGKRWPTGVMLMVVVLFAFLYSWPIQALLPTYLKTDLAYNPHTVANVLFFSGFGAAVGCCVGGFLGDWLGTRKAYVCSLLASQLLIIPVFAIGGANVWVLGLLLFFQQMLGQGIAGILPKLIGGYFDTDQRAAGLGFTYNVGALGGALAPIIGALIAQRLDLGTALASLSFSLTFVVILLIGLDMPSRVQRWLRPEALRTHDAIDGKPFSGAVPFGSAKNDLVKTKS

SEQ ID NO: 2 - protein, 508 aa, Yersinia enterocolitica subsp. palearctica serotype O3
MSTSVQPQTDKPPRGDKPLRWYKQLTPAQWKAFIAAWIGYALDGFDFVLITLVLTDIKQEFGLTLIQATSLISAAFISRWFGGLVLGAMGDRYGRKLAMITSIVLFSFGTLACGLAPGYTTLFIARLIIGIGMAGEYGSSSTYVMESWPKNMRNKASGFLISGFSIGAVLAAQAYSYVVPAFGWRMLFYIGLLPIIFALWLRKNLPEAEDWEKAQSKHQQSKQAKDRNMVDILYRSKLSYRNIALTLFAAAALYACFTGMVSTPVVVLLGILCAAVFIYFMVQTSGDRWPTGVMLMVVVFCAFLYSWPIQALLPTYLKMDLGYDPHTVGNVLFFSGFGAAVGCCVGGFLGDWLGTRKAYVTSLLISQLLIIPLFAIQGSSILFLGGLLFLQQMLGQGIAGLLPKLLGGYFDTEQRAAGLGFTYNVGALGGALAPILGASIAQHLSLGTALGSLSFSLTFVVILLIGFDMPSRVQRWVRPSGLRMVDAIDGKPFSGAIGPVGASVVTQK

SEQ ID NO: 3 - protein, 494 aa, Buttiauxella brennerae ATCC 51605
MNNTTQQIPWYRHLTKPQWKAFSAAWIGYLLDGFDFVLIALVLTEIQSEFGLTTIQAASLISAAFISRWFGGLVLGAMGDRYGRKLAMITSIVLFSFGTLACGFAPGFTTMFFARLLIGMGMAGEYGSSATYVIESWPKQMRNKASGFLISGFSVGAVIAAQVYSVVVPVWGWRALFFIGILPIIFALWLRKNIPEAEDWKEKFADKKPTKTMVDILYRGEHRLINIGLTIVVAAALYFCFAGNLGNALLIAVLGLICAAIFISFMVQSSGKRWPTGVTLMIVVFFAFLYSWPIQALLPTYLKTELAYDPATVAHVLFFSGFGAAVGCCVGGFLGDWLGTRKAYVCSLLASQLLIIPVFAIGGGNIWVLGLLLFLQQMLGQGISGILPKLIGGYFDTEQRAAGLGFTYNVGALGGALAPVIGATIAQRLDLGTALASLSFSLTFVVILLIGLDMPSRIQRWIHPEAVRTHDAIDGKPFSGAVVSGSHKTKLIGN

SEQ ID NO: 4 - protein, 490 aa, Escherichia coli KTE75
MDSCIQKKQVWYKHLSPRQWKIFGAAWTGYLLDGFDFVLISLVLTEVQHEFGLTTIEAASLISAAFISRWFGGLAIGALSDKMGRRMAMVLSIVLFSLGTLACGLAPGYAVMFIARIVIGLGMAGEYGSSVTYVIESWPVHLRNKASGFLISGFSIGGGLAAQVYSIVVPLWGWRSLFFVGMLPILFAFYLRKNLPESDDWQKRQQENKPVRTMVDILYREKNKYINILLSCIAFACLYVCFSGVTANAALITVMALCCAAVFISFIYQGMGKRWPTGIMLMLVVMFCFLYGWPLQAFLPTWLKVDMQYSPETVALIFMLAGFGSAAGSCIGGFMGDWLGTRKAYVISLLIGQLVIIPVFLVDRDYVWLLGLLIFTQQVFGQGIGALVPKIISGYFNVEQRAAGLGFIYNVGSLGGACAPILGAVVASHTSLGTAMCSLAFILTFVVLVLIGFDMPSRVQRWIHPEAALEYDTVDGKPFYGARKKNVAGE

SEQ ID NO: 5 - protein, 572 aa, Mycoplasma alligatoris A21JP2
MVDTANIVKEASKKLIAKSNFTALDFVVMAAYILAILGMGIWFSIAAKKAKETNSDDYFTGGSKIPTWVIGFSIWATTLSAITFIALPANAYNNGWLFAFSQLTIIIFAPVLIKFVIPFFRRMKESTAYAYLHARFHYALRAFSSILFILFHIFRMGIVLYIPANALSLVVPLDPWVLVIIMGVVVVITTYLGGQRGVLWQDAIQGIILLLGIVLIIVFSLYNIFGTGNGTNGKESIKYAQLLKASDLHVTLVGIGIPLILVSRYIEVIYTYVGGQDVVQRYKASKKTTNINKSIWINAGLALITILLFYGAGSALYTYYDGMAITQIDPAIKHTNQLVPYFIFTVLPTGIAGLIISAVFAASQSTMSSSLSSLVNSIIVDFVKVFAKKFSNNEKKLFLLSKILIGAFGAFGVICGIAFTLTNLGDIINYFLGVVGLFGAPTAAIFMLGMFTKRTSWISALIGMVTGFLVGLIIWIFSTPDFVGGKEFVKFNSGWVGVFGFFVTLLVGYLTSFIFPNKKNITNLSWHTLTPEFKELIALEKTIEKAAKKKQNADKEILRYDELIRKLEMSAE

SEQ ID NO: 6 - protein, 570 aa, Mycoplasma capricolum subsp. capricolum
MNSLILFTEKTTNAAIKSFHWVDYVVLVIYLLFTLSIGLYFQLKSKLQKANNTDEYFKAGGKTPGWVAGFSIYATTLSAITYMATPAKAFSSDWLFAFGNLTIFIATPLLIRFIIPFFKKLNDVSGYGFLEKRFSYFLRVSASLMFILFHVIRVGLIIYLPTIALTAVTNINPYLIAIIIGVFCVISTVLGGLNGVIWSDFIQAVVLIGGILLAIIFALVKIENLSEVNNIVINNHKLLEKESIIPQNWAKPWILVLFFGQLINTFYQYIGSQDVVQRYQSNQSFKQVKKGLWTNAILALITILLFYGMGTLLYAFYVQKTGLTNIEDIMRSIGIKANNQILPYFIVTVLPVGISGLIIAGIYSASMSTISSSLHSAATCIVEDILVRIKPNLKDKEKMYWAKGLILGIGLLGTIASIILIISKADNLLDLFAAVIGLFGTSVTVIYLLGIFTKRTSNIGAILGSISSFIIVFAIFIVNKTSKTTVVSDFYGIILAFIVGLLLGYLSSFIFKNKKIDKLEGLTIHTFSKQDFNKMIEDGEKEWQEIKANENQFKKDLIKKIKQKKVAKSN

SEQ ID NO: 7 - protein, 510 aa, Staphylococcus aureus
MKEVGFGTLNWVAVIIYLLAMLFIGVYFTKRASQSTNSFFTASGRLPSWVVGFSIYATTLSAITFMSTPEKAFLTDWSYIAGNIAIVAIIPLLIYFYVPFFKKLKVTSAYEYLEARFGPSIRVIGSLLFVVYHLGRVAIVIYLPTLAITSVSDMNPYIVASLVGLLCILYTFLGGFEGVVWSDFIQGVILLGGALVIIILGVVNIKGGFGTVFADAIEHKKLISADNWKLNTAAAAIPIIFLGNIFNNLYQYTASQDVVQRYQASDSLKETNKSLWTNGILALISAPLFYGMGTMLYSFYTHEAVLPKGFNTSSVVPYFILTEMPPFVAGLLIAAIFAAAQSTISSSLNSISACISIDIKQRFFGKGSERHEVNFARFIIIIAGIFGFGMSLYLIASNSNDLWDLFLFVTGLFGVPLAGVFAVGIFTKRTNTFGVICGLILGIIFAYVYNGVGKGNSPFYVSTISFTVAFVFAYILSFIVPSKHKKDITGLTIFEKDKPSTYISKTATKK

SEQ ID NO: 8 - protein, 517 aa, Gemella sp.
MDKIGFGTLNSIVLAIYLIAMLAIGVYFTKKAGESTDEFFKAGGNIPSWAVGFSIYATTLSAITFMATPEKAFNGDWTYAAGNFSIIAIIPILIHYYIPFFRKLNVTTAYEYLEERFNPSVRVVGSVLFSLFHIGRVAIVIYLPTVAITSVSTLNPFLVCAIIGLLCVVYSFLGGVEGVIWTDVIQGIILLGGAVLVIILGAYHVGGFGQITQDALANGKIVTAEKFSWSLTASTIPIIFIGSVFNSLQQYTASQDVVQRYSTTESVKETNKSIWTNGLLAFISIPIFYGMGTVLYSFYKGSHAVTKLPDFMNAEVVGKAANTTALVPYFILTALPAGVAGLVIAAILAAAQSTIASSLNSISACITVDIKQRFFGLSKDAKQDVLLARIIIIIAGVLGTIAALYFVNSNQKETWDLFLKYIGLLGVPLAGTFALGIFTKRANGAGALLGLVVAGLVCWYIQDFTTYAKQSPFIFSGFSFLSALVFGYICSFFFKSTKNITGLTIHTKDQEYVKEAK

SEQ ID NO: 9 - protein, 500 aa, Clostridioides difficile ATCC 9689 = DSM 1296
MVGFTWIDFAILVVYLLAVLFAGLLFSKKEMKGKEFFKGDGTIPWWVTSVSIFATLLSPISFLSLAGNSYGGTWILWFAQLGMFIAIPLTIKFFLPLYSRLEIDTAYEYLEMRYESKGLRVLGALMFIIYQIGRMSIIMYLPSVVLAGLTGIPVNVLIVVMGVIAIIYSYTGGLKAVLWTDFIQGMVLIVGVTGTLFYLIASINGGFGTVMDTLTTGHKFLASNEVMFDKNILSTSAFIIFIGAGLNTFSSYVSSQDIVQRFTTTTDIKQLNKMTYGNGVLSMGLATVFYLIGTCLFIYYTQNPDLAQTVQQDQVFASYIAYELPVGITGILLAAIYAASQSTLSTGLNSVATSWTLDIQTIITKDISMEKQTKIAQYISLGVGILSIAVAIVLANGEIKSAYEWFNSFMGLVLGVLAGIFVLGAFCKKATKMGAYVGFIVSAILVVYLKYRMPEVTSWAYSLITITTSVVVGTIVSAIEAKVKAKVSLPKAETTVYYEK

SEQ ID NO: 10 - protein, 513 aa, Peptostreptococcus russellii
MDKIGFGFANGVVLVLYLLAMTAIGGLFAKKAGKNTDAFFKASGSLPSWAVGFSIYATTLSAITFMATPEKAFNGDWAYAAGNLSIIVIIPILIRFYVPFFRKLNVTTAYEYLEARFNPAMRVFGSVLFSLFHIGRIAIVIYLPTVAITSVSTINPYLVCGAIGILCVIYSFLGGMEGVIWTDFIQGVILIGGALLVIAVGVFNVGGFGNVAADAVAHKKILSNETLQWSFTAATVPIIFLGSIFNSLQQYTASQDVVQRYNTTKNQKETNKSLWTNGLLAFITIPIFYGMGTVLYSFYKGAHAAGSLPNFMNADVVGAAANTTALVPYFILTSLPAGVAGIVIAAILAAAQSTISSSLNSISACVVVDIKQRFFGKGNDQEDVRLARLVIIASGLLGTIAALYFIKANQKETWDLFLKFIGLFGVPIAGIFALGIFSKRANGTGALIGLLASAFSCYWIQGNTTWAANPFVFSIFSFLSAVVFGYIFSLIFKKDKKNIDGLTIYTKDQEYIG

SEQ ID NO: 11 - protein, 511 aa, Bacillus obstructivus
MEKISFGTANWMVLILYLLAMLAVGVYFTKKSSKNTDAFFTAGGKIPAWAAGFSIYATTLSAITYMSTPEQAFLSDWSYSAGNLTIFAIIPILIYFYVPFFRKLNVTTAYEYLEERFGVSMRFIGSLLFTLFHIGRVAIVIYLPTLAITLVSDINPIIIATAVGGLCIIYTFLGGMEGVIWSDVIQGIILLGGAILLVVLGINAIDGGFTTVIQNAMDNDKFLSAKNFQFGGVAAAIPIIFLGSIFNNLHQYTASQDVVQRYQTTSSIKETNKSLWTNGILALITIPVFFGLGTVLFSYYTNVEALPEGFNTSALVPYFIVSSLPAGVAGLLIAAIFAACQSTISSSLNSISACVTVDFKQRFFGKDDKKDVWLARIIVIVAGILGMAAALYLISTNRAETWNLFLSITGLFGVPIAGIFALGIFTKRANAPGVLIGLFLSALTSYFVQKTNLTPFTVSIVSFFSSFILGYIVSFLFPKYNKNIIGLTIYTKNESYTKTKGSDPHRITAVK

SEQ ID NO: 12 - protein, 510 aa, Staphylococcus aureus (strain MW2)
MKEIGFGTLNWVAVIIYLLAMLFIGVYFTKRASQSTNSFFTASGRLPSWVVGFSIYATTLSAITFMSTPEKAFLTDWSYIAGNIAIVAIIPLLIYFYVPFFKKLKVTSAYEYLEARFGPSIRVIGSLLFVVYHLGRVAIVIYLPTLAITSVSDMNPYIVASLVGLLCILYTFLGGFEGVVWSDFIQGVILLGGALVIIILGVMNIKGGFGTVFADAIEHKKLISADNWKLNTAAAAIPIIFLGNIFNNLYQYTASQDVVQRYQASDSLKETNKSLWTNGILALISAPLFYGMGTMLYSFYTHEAVLPKGFNTSSVVPYFILTEMPPFVAGLLIAAIFAAAQSTISSSLNSISACISIDIKQRFFGKGSERHEVNFARFIIIIAGIFGFGMSLYLIASNSNDLWDLFLFVTGLFGVPLAGVFAVGIFTKRTNTFGVICGLILGIIFAYVYNGVGKGNSPFYVSTISFTVAFVFAYILSFIVPSKHKKDITGLTIFEKDKPSTYISKTATKK

SEQ ID NO: 13 - protein, 508 aa, Staphylococcus sp. HMSC070A03
MQQVGFGTWNWVAVITYLVVMLIIGAYFTKRAGQSTDSFFTASGRLPSWAIGFSIYATTLSAITFMSTPEKAFLTDWSYIAGNIAIVAIIPALIAFYVPFFKKLRVTSAYEYLEARFGPSVRVLGSLLFVVFHLGRIAIVIYLPTLAITAVTDINPYLVASLVGILCILYTFLGGFEGVVWSDFIQGVILLGGALMIIVIGAMEIKGGFGTILHDALAEKKLLSVDNWKLNTTAAALPIIFLGNIFNNLHQYTASQDVVQRYQASDSIDETKKSLWINGLLALISAPIFYGMGTMMYSFYEHEAALPQDFNTSSVVPFFILTQMPPFVGGLLIAAIFAAAQSTISSSLNSISACISEDIKDRFFSKGTDKGNVRFARWTIIIAGLLSYGIAIYLIAADSNNLWDLFLLVTGLFGVPLAGIFAVGIFTKRANTFGVLVGLFTGVIVAYLLKGIGGANSPFYVSIISFFIAFLFGYIVSLFVPAKYTKDITGLTIYDKDKPSTYISKTRQ

SEQ ID NO: 14 - protein, 509 aa, Cetobacterium ceti
MNWHWLNWVVLCLYFLGVVYIGVYFSKKNNSSEDYFTASGRIPAWVTACSIYSTALSSISFIAIPASVYKHGWIMGMAPLGIIIMIVWAGYAFVPFFRKVRVITAYEFLEKRFDRSFRLVGSLSFILFHVIRIAIVLYLPTLALIAALPGVNPILLTAIVGFLCVVYTSMGGIEAVVWSDAIQTIVLLFGAFLIIIIGFMSVPSGVSSFDVLENAGKIFAPGTFDFNLAGKTFWGILIGGFLNSIYSYVGSQDIVQRYNTTKNIKEAQKSLYMNVPLALTSVVIFVGMGSALFLFFKFHTVLPEHIDGNAILPYFVVHNVPLGFSGLVIAAIFAAAQSTVSSSLNSVATCITSDIVKPMMKNLTDKHELKIAKYCSWVVGIISSLLSLRFLSAGQGDMFLYFQAITGLLGGPIAGLFLVGIFTKRVNGKAAWIGFICSVLVAIYIGNPAGLLTKFIPGYEPPHIFEILISLVIIGSCVVPAYIASFFTGRPDEDKVYHLTYESTKLINE

SEQ ID NO: 15 - protein, 509 aa, Staphylococcus argensis
MHQVGFGLWNWVAVIVYLVAMLLIGSYFTKRASKDTDSFFTAGGKLPSWVVGFSIYATTLSAITFMSTPEKAFLTDWAYVAGTITIIAIVPILNYFYVPFFKKLRVTSAYEYLEARFGPSIRLIGSLLFVLFHLGRVAIVIYLPTLAITSVSDMNPYIVATLVGVLCILYTFLGGFEGVVWSDFIQGVILLGGAFFIILLGALQIHGNFGTIWHDAMEHKKILSADNFVINSASAAFPIIFIGNIFNNLHQYTASQDVVQRYQASESMKETRKSLWTNGLLAILSAPLFYGMGTMLYSFYQHAGTLPKDFNTSSVVPYFILTQMPPFVGGLLVAAIFAAAQSTISSSLNSISACISVDIKHRFFGKGKEKDEVKFARLMIILAGLFGYGMSMYLIFAKSNNLWDLFLLITGLFGVPLAGIFAVGIFTKRTNTFGVIVGLIAGILVAYLLNDKSNPSAPFYVSIMSFTIAFIISYVVSLFTPVKTKDITGLTIYDKDKPSTYISKVKTKG

SEQ ID NO: 16 - protein, 506 aa, Staphylococcus schleiferi
MHTIGFGFWNWVALLCYLLLMLGIGAFFTKRAGKDTNSFFTASGRLPSWAVGFSIYATTLSAITFMSTPEKSYLTDWSYIAGNVAIVAIIPLLIYFYIPFFKKLKITSAYEYLEARFNPAVRVLGSLLFVLFHLGRVAIVIYLPTLAITAVSDINPYLVASLVGVLCIVYTFLGGFEGVVWSDFIQGVILLGGALVIIIIGIVHIDGGWGTVVNEAVDKQKLISADNWKFTAASAAIPIIFLGSIFNNLQQYTASQDVVQRYQASESLKETSHSLWTNGLLAIISAPLFYGMGTVLFVFYTHHTTLQKDFNTSSIVPYFILTEMPPFVAGLMIAAIFAAAQSTISSSLNSMAACLSVDIKQRFFGKKSEEAEVRFARLATIVVGLLGMLISLYLIAANSNDVWDLFLLITGLFGVPIAGIFAVGIFTKRTHGVGVIIGIIIAVIVSYFLQGVGGAGSPFYVSIIAFITAFVTAYIASLIIPAPNKDISGLTIFDKNGAITYERKVK

SEQ ID NO: 17 - protein, 507 aa, Alloiococcus otitis ATCC 51267
MSGQGFTWIDLVILIVYLIFVLFFGNYFAKKEMIGKEFFKSDGTVPWYVTSVSIFATLLSPISFLSLPGNSFGGTWILWFAQLGMIIAIPIAIAFFLPVFAKLDIDTAYDYLERRFDNRGLRVVSAIIFIIYQIGRMSIIIYLPSITLAELTGLNVNILIIAMGAIAIFYSYTGGLKSVLFSDFVQGIIVLVGVTLSLIILINGIDGGVGTIAETLADGKFIAPDEQWFSPTIFEDTVFLVFVGAGMNTLTQYISSQDIVQRFTTTTNTKDLTKMMLTNGVLSLFIATILFLIGTGLFIFYGQAGIASGMDQDLAYGYFIAYQLPIGVTGVLLAAIFAASQSTLSTGLNSVATSVALDIVEVFDTKGKYDYKSKTKIARGVTIVVGLFAIVVSMIMVQSNIVSAYEWFNGFVGLVLGVLGGVFALGIFTKRGNAVGAYIGFLVSAVVMIIIEFVIPEEINISIWANSLVSIIVAFVAGYLGSVIYSAISSKDSKREDYSSILDFNKN

SEQ ID NO: 18 - protein, 507 aa, Gracilibacillus dipsosauri
MDKVGLGAINWIVLIVYLVSMLGVGVYFTKRSSKNTDAFFTASGKIPAWAAGFSIYATTLSAITFMSTPEQAFLSDWSYAAGNLAIFAIIPILIYFYIPFFRKLNVTTAYEYLEERFGPSLRVVGSLLFVLYHIGRVAIVIYLPTLAITSVSSINPIIIASVVGGLCIIYTFLGGMEGVIWSDVIQGILLIGGAILVLVLGILTINGGWSTVINDALANDKLITADNFKLGTAAAAIPIIFIGSIFNNLHQYTASQDVVQRYQTTKSIKETNRSLWTNGILALITIPIFYGMGTVLFSYYSNMETLPEGFNTSAIVPYFIVTTLPVGVAGLLIAAIFAACQSTISSSLNSISACITVDFKQRFFDKAFRLNDVIIARLVIIVTGTLGMIAALYLVSTNQAETWNLFLAMTGLFGVPIAGIFALGIFTKRANAPGVLIGLILSAITSYFIQQTSFSPFAVSVMAFFSSYIFGYVASFFFPAYNKNTTGLTIYTKNDDFIKSDVKQNIS

SEQ ID NO: 19 - protein, 502 aa, Lactobacillus versmoldensis
MTKVGFGTLNWVVLLVYLVAMLGVGVYFTKKASKNTDAFFKAKSSIPAWAAGFSIYATTLSAITFMSTPEQSFLNDWAYSVGNLSIFLIIPILIKYYVPFFRKLKVTTAYEYLEERFSPVMRVIGSLLFILYHIGRVAIVIYLPIIAITSVSNINPILIACVVGGLCIIYTFLGGIEGVIWSDVIQGMLLLGGALLIIIVGIVSIKGGFGTVVSDAVSGDKFFSVKDWNPGDLTKFIPLIFAGQFFNTLYQYTGSQDVVQRYQTTATQKETNKSLWTNGFLALITIPLFFGMGTVLFSFYKNVGTLPHGFNTSAIVPYFVVTRIPAGIAGLVIAAIFAAAQSTIASSLNAISSCVVTDFKQRFFNHRLKSISDVTLARIIIIVTGLFGLGMSIYLLIGNASKTWDLFLTVTGLFGVPIAGIFAAGIFTKRSNTFGAVLGLILSVIATVFVQQNSDSSLIVATVAFITSFIFTYLISFFAPKKYVHNVVGLTASTVKETYVKS

SEQ ID NO: 20 - protein, 504 aa, Lactobacillus sp. LL6
MTKTGFGMWNWIVLCAYLLAMLGVGAYFTKRASKSTDSFFKADGRIPAWAAGFSIWATTLSAITFMSTPEQSFLTDWGYAIGSLTIFVIVPILIAYYVPFFRKLKVTTAYEYLEKRFGPVMRALGSGLFMLYHIGRVAIVIYLPILAITSVSNINPILIAVGVGGLCIIYAFLGGIEGVIWTDVIQGFLLLGSALLICIMAIIHTGGLGHVADIAVANHKFFSAADWNPSDLAKFIPLIFAGQFFNSMYQYTASQDVVQRYQTTPSLKATAKSLWTNGVLALITIPLFFGMGTLLYCFYHTNGTLPKGFNTSAVVPYFVIKEIPAGLAGLIIAGIFAAAQSTIASSLNAISSCAITDFKQRFFNNKFKNISDVALARWIIIIAGVISLSIAIYLLMGDASQTWNLFLTVTGLFGVPIAGIFAAGIFTQSANRFSVVVAVILSAAITYWIQTLNVSTFVTPCVSFVLSYLISWIVGLMTPQYNHDITGLTARTIKDKVSAQEELE

SEQ ID NO: 21 - protein, 503 aa, Agrilactobacillus composti
MTKAGFGMWNWIVLIIYLLAMLGVGVYFTKKASKNTDAFFKAEGKIPAWAAGFSIYATTLSAITFMSTPEQAFLNDWAYAIGNFSILLIIPLLVKFYVPFFRKLNVTTAYEYLEERFSPVMRAISSLLFMLYHIGRIAIVIYLPIIAITSVSNINPIVIALAIGPLCIIYTFLGGIEGVIWSDVIQGFLLLGGALLVCILGVTYIKGGFSTVFQDISTHQKVLSGADFNVHDLSKFVPLILAGQIFNSLYQYTGSQDVVQRYQTTGSMKEAKKSLYTNGLLALITIPLFFGMGTILFSFYQHAGSLPKGFNTSAVVPYFIITEMPAGVAGLVIAGIFAAAQSTIASSLNAISSCAVTDFKQRFFDDKLKGLKDVTLARIIIIIAGVLSLSVAIYLLMGNASQTWNLFLTVTGIFGVPIAGIFAVGIFTKRANTTGVLIGLVCSVALSFYVQSLNISSLLVACSAFISSFILSYGISLLVPTQLKNITGLTVSTIKETYVREGK

SEQ ID NO: 22 - protein, 502 aa, Lactobacillus crispatus
MSKVGFGFWNWVVLIAYLLVMLGIGAYFTKRASESTDSFFKANGKIPAWAAGFSIWATTLSAITFMSTPEQAFLNDWGYTFGTFGIFLLIPVLIKWYVPFFRKLKVTTAYEYLEHRFGPVMRALGSALFMLYHIGRVAIVIYLPILAITSVSNINPVLVAVLVGLLCIIYAFLGGIEGVIWTDVIQGFLLLGSAILICGLAIFYTGGFGNVFHIALGAHKFYSAADWNPNDLAKWIPLIFIGSWFNNMYQYTASQDVVQRYQTTPSMKATAKSLWTNGWLALSTIPLFFGMGTLLYCFYHSAGSLPHGFNTSALVPYFVITKIPAGLAGLIIAGIFAAAQSTIASSLNAISSCFVTDFKQRFFDKKWKINDVKLARIVILVAGAISLSIAIYLLLGNASQTWNLFLTVTGLFGVPVAGIFAAGIFTKSANRFSVIIGVIASAALTYWMQLGSPSPFLTSTVSFVSSFLVAWLVGLITPKFGHDVTGLTVKTINEKVDTSKNK

SEQ ID NO: 23 - protein, 502 aa, Staphylococcus pasteuri
MNEVGFGTGNWIALIIYLLATLLVGVYFTKRAGQDTESFFKAKGRIPAWAVGFSIYATTLSAITYMGTPERAFNTDWSYAAGNIAIIAIIPLLIYFYIPFFRKLDVTTAYEYLEARFGIVLRVIGSLAFTLFHIGRVAIVIYLPTLAITSVSDINPYLIAALVGLLCMVYTFLGGIEGVIWSDVIQGIILIGGAVAVIFLGAFQIDGNFATVAQEAMQDKKLISVDNWKFGTAAAAIPIIFIGSIFNNLHQYTASQDIVQRYQTTGSVSSTNKSLWTNGILAFITIPIFYGMGTVLYSFYNNVSTLPEGFNTSAIVPYFILTQIPPFVGGLLIAAIFAAAQSTISSSLNSISACVIVDLKQRFSKNNNSEHDVLFARIVIIIAGIFGTIASLYLINQETNETWDLFLLVTGLFGVPIAGVFAVGIFTKRANSYGVIIGLIVAAIVSYFIGKTEITPFTVSVIGFFIAFIVGYLASLPFSKHNKNIVGLTIYTINDQYVKDTK

SEQ ID NO: 24 - protein, 501 aa, Lactobacillus salivarius cp400
MEKVGFGTLNWIVLVLYLLAMLAVGAYFTKKASKNTDSFFTAEGKIPAWAAGFSIYATTLSAITFMSTPEQAFLNDWAYSIGNLAIIAIIPILVKYYVPFFRKLKVPTAYGYLEERFGPVMRILGSLLFMLYHIARVAIVIYLPIIAITSVSDINPIIIALFVGGLCIIYTFLGGIEGVIWSDVIQGILLLGGALLVIILGAHYIDGGWATVASDAITHHKLISGFDMKISDLSKFLPLIFAGQFFNSLYQYTGSQDVVQRYQTTATMEETKKSLWTNGILAFITIPIFYGMGTVLFSFYNHAAMLPKGFNTSAVVPYFIVTKLPAGIAGLVIAAIFAAAQSTIASSLNAISSCGIIDFKQRFFNDKFKNVSDVALARWIIIISGLFSLAVAIYLLVGNTSETWNLFLAVTGLFGVPIAGIFAAGIFTEKANTVSAVIALIVAAGLTYYVQSLNISPFITSSVSFLTSFVVSYLVGLVTPQYRKDVTGLTVRTIDVEYTKK

SEQ ID NO: 25 - protein, 500 aa, Lactobacillus apodemi
MEKVGFGLTNWIVVVLYLVGMLLVGAYFTKRSSKDTDAFFKAGGKIPAWAAGISIFATTLSSITFMSVPERAFLSDWSYAIGSLVIIPIIPILNHFYVPFFRKLKVTTAYEYLEARFSPLIRALSSLLFMIYHIGRVAVVTYLPVLAVASVTDINPLLVAACVGIMCIIYTFLGGIEGVIWSDVIQAFVLIGGALLIIFIGAFSIDGNFATVAHTAASNHKILSGADFKLSDLANFVPLIFVGQFVNSLYQYTGSQDVVQRYQTTKSLKETIKSLWTTGFLGILTIPLFFGMGTILYTFYQNVATLPKDFNTSAIVPYFVITQLPAGIAGFVIAGIFAAAQSTIASSLNSISACFVTDFKQRFFNDKFTKVSDVSLARIVIIVAGVISTAITIYFTLGNTSDTWNVFLTVSGLFGVPVAGLFALGIFTKRANARGVLLGFVLSVVLAFYIDSLQISSLLVATVSFTAAVVFGYFASLLFPNNNKDNRGLTIYTLKDKVDD

SEQ ID NO: 26 - protein, 515 aa, Selenomonas sp. oral taxon 478
MHEGFTWIDTAVLIIYLLGVLLAGLYYSKKEMKGKEFFKGDGTIPWYVTCVSIFATLLSPISFLAIPGNSYHGSWIFWWAQLGMLFAIPLTIRYFLPIYSRLEIDTAYHYLQKRFESGKLRILGALMFIVFQLGRMSIIMYLPSVALAEVTGIDVNLLIVVMGVIAIIYSYSGGLKAVLYTDFIQGTVLIVGIALSLVVMIGSIHGGWGTVWDTLTTGHKFLLENEVWFSPDIVSSSVFITFIGGGLVTFASYVSSQDIVQRFTTTTDMNQLRKMTLGNGVISIFAATVFFLVGTALFVFYQQNPGLLMTDRRDLVLAAYITYELPTGLTGILLAALFAAAQSTLSTGINSVATSWVLDIQSVLHPEMPMKKQTRIAQFISLGIGILSIVVAIIMAGSDIRSAYQWFNSFIGLALGALAGVFVLGAFSRHANAAGAVAGFLVSTAVVIYLKYFVPSVSFWSYTLITIALTLGVGTIVSRLTAPGYTAPAGTTVYDTRGASRCDGQEDAAVCHVHK

SEQ ID NO: 27 - protein, 501 aa, Paeniclostridium sordellii
MGGFTVVDFVILVVYLLAVLLAGLHFSKREMKGKEFFKGDGTIPWWVTSVSIFATLLSPISFLSLAGNSYGGTWILWFAQLGIFIAIPLTIKFFLPLYSRLDLDTAYQYLEIRYNSKGLRVLGALMFIVYQIGRMSIIMYLPSVVLAELTGISVNILIIAMGVIAIIYSYTGGLKSVLWTDFIQGMVLIVGVTGTLIYLIANINGGVGTVVETLTSGQKFLAQKEVIFDPNILKTSAFIIFVGAGLNTFSSYVSSQDVVQRFTTTTDLKQLKKMTYGNGVLSIVVATIFYLIGTCLFVFYSQNPELAQTVKQDQIFASYIAYQLPVGITGILLAAIYAASQSTLSTGLNSVATSWTLDIQTLINKDMSFERQTKIAQYISLGVGILSIGVSIVLANGEIKSAYEWFNSFMGLVLGVLAGIFVLGAFCKKATKLGAYIGFAVSAVLVIYLKYKAPEVSSWAYSLITITTSVVVGQISSLIQLKITNKVNKVDSESTIYYESN

SEQ ID NO: 28 - protein, 509 aa, Butyribacterium methylotrophicum
MQGFTTIDLIILIVYLAAVLVIGLAFAKKDMQGKEFFRGDGTIPWWVTSVSIFATLLSPISFLSLVGNSYAGTWILWFAQLGMLIAIPVTIRFFLPMYSRLNIDTAYEYLQLRFDSRGLRILGAIMFIIYQIGRMSIIMYLPSMVLANLMNINVNILIILMGIVAIAYSYAGGIKSVLWTDFIQGCVLIAGVVFALIFLICSIDGGMGAVMGELANGKFLSDTQMLIDPKIWKDSVLIILVGAGFNTFSSYISSQDIVQRFTTTTDVKKLNKMMLTNGALSIFVATVFYLIGTGLYVFYGQNPALAQTAQQDQIFGSFIAYQLPVGITGILLAAIYAASQSTLSTGLNSVATSWTLDIQTMVSKKEMSFKNQTRIAQFITLGVGIIAIVVSMVLANGEIKSAYEWFNGFMGLVLGVLGGTFVLGAFTKKATDIGAYAAFAVSAAVVIYLKYNVPEVSIWAYAIISIFVSLIVGYIISAVHRAVSKKSFNYDDRTTIFFKKDKQETGEKA

SEQ ID NO: 29 - protein, 495 aa, Alkalihalobacillus pseudofirmus
MEGQFAIIDYIILFGYLLFILYVGIAVAKKEIQGKEFFKGDGSIPWWVTSVSLFATLLSPISFLSIAGNSYIGTWQLWVAQLGLLIAVPVAIIFFLPVYRNLNLDTAYEYLERRFDKNLRMLGSFLFIIYQIGRMSIIMYLPAIALAYVTGLSPILIILFMGVIATIYSAFGGIKSVLWTDFIQGVVLIGGGIFALIVLLFSIDGGMSEVFKVGVEDNKFFSDVVVFDPNFLKDSLFIIFLGAGLSTAFSYISSQDMVQRYLTTNNLKEMNKMTIFNGVLSISTATLFFFIGTALYVFYTQQGGPLPTGKSDLIFANFIVNQLPAGISGLLIAGLFAAGQSTLSTGLNSVATSWTLDIQKVLKPDLSDEKGTRIARIVSIVVGVFSITFAIVLAHSDVSSAYAWFNGLMGLVLGIIGGTFTLGVLTKKANAKGAMVGFIATTLVAIYVSYFTDTTFWAYSLINLVTSMVFGYIFSFIFSGKSKAEDQPLTYYDHK

SEQ ID NO: 30 - protein, 513 aa, Streptococcus mitis (strain B6)
MGTTGFTIIDLIILIVYLLAVLVAGIYFSKKEMKGKEFFKGDGSVPWYVTSVSIFATMLSPISFLGLAGNSYAGSWILWFAQLGMVVAIPLTIRFILPIFARIDIDTAYDYLDKRFNSKALRIISALLFIIYQLGRMSIIMYLPSAGLSVLTGIDINILIILMGAIAIVYSYTGGLKSVLWTDFIQGVVLISGVVVALFVLMANIKGGFGAVAETLANGKFLAANEKLFDPNLLSNSIFLIVMGSGFTILSSYASSQDLVQRFTTTQNIKKLNKMLFTNGVLSLATATVFYLIGTGLYVFYQVQNAGSAASNIPQDQIFMYFIAYQLPVGVTGLILAAIYAAAQSTISTGLNSVATSWTLDVQDVISKNMSDDRRTKIAQFVSLAVGLFSIVVSIIMAHSDIKSAYEWFNSFMGLVLGLLGGVFILGFVSKKANKQGAYAALIVSTIVMVYIKYFLPPTAVSYWAYSLISISVSVVSGYIVSVLTENKVSAPKYTTIHDIREIKADSSWEVRH

SEQ ID NO: 31 - protein, 512 aa, Aerococcus viridans
MSKTGFTTIDFAILVVYLISVLFAGLLFSKKEMKGKEFFKGDGTIPWYVTSVSIFATLLSPISFLTLPGNSFAGSWILWFAQLGMIVAIPIAMRYFLPIYAKLDIDTAYDYLERRFDSKGLRVIGALMFVIFQLGRMSIIMYLPSAALATLTGIDINILIIVMGAIAIVYSYTGGIKSVLWTDFIQGVVLLIGVSISLFFLARDIDGGFGEIFNTMGNNKFLGSEEAIFNPNFLKTSVFMMVIGAGLNTCSSYVSSQDIVQRFTTTTDTKKLNKMMVTNGVLSIFIATAFYLIGTGLYVYYKVQNPADPGASIAQDQIYAYFIAYRLPVGMTGILLAAIYAASQSTLSTGLNSVATSWTLDIQEVITKDMDDKRKTALARYISLGVGIFAIVISIIMAHSNIQSAYEFFNSFMGLILGVLGGTFALGIFTKKGNKYGAYAALISSSAVMVFIKYGLPAEAVSIWSYSMISIFVSLVVGYIVSLLVPVKTTAPKYTTVSDISEIKADSSTPVN

SEQ ID NO: 32 - protein, 509 aa, Anaerobiospirillum thomasii
MFDNLHGFTWIDTVILIIYMIAVLLAGFYFSKKEMKGKEFFKGDGSVPWWVTSVSIFATLLSPISFLALAGNSYGGTWLLWFAQLGMIIAIPLTIKFFLPVYANLGIDTAYHYLEIRYGSRALRVLGAIMFVIFQMGRMSIVMYLPCLALAKLAGIDVNILIIGMGAIAIVYSYTGGLKAVLWTDFIQGVILIGGIGLTLVYLIWRLDNGLGDITYSLTEGGRFLANGEDWINWSNLLGTSILLTIVGGGLSTFCSYVSSQDVVQRFTTTTDVKQLNKMTIGNGVLSIVCASIFYLVGTALFIFYEQNPELITDTFKGSQDQVFAYFITYELPVGVTGIILAALYAASQSTLSTGINSVATSWVLDIQSQITPNMTSERQTKIAQYVSLGVGVFAILVAMYLATASIRSAYELFNSFLGLALGALAGIFVMGAFTRKTSALGALCGFAVSILVVLYCKYYVPSINFWSYAVIAIIITTVVGVIVSKIQAAVTQKEYIAPEGSTFMTAKK

SEQ ID NO: 33 - protein, 507 aa, Clostridium haemolyticum NCTC 8350
MKTSFEFVDYLVLIGYLLFVLIIGIKVAKKEMKGKEYFRGDGTIPWWVTAVSLFATMLSPISYLALAGRSFKGDWSSWVGQLGIFVAVPLTIIFFLPVYKKLNIDTAYEYLEKRFDKRLRLLGSLMFIVFQIGRMSIVMYLPALALSIVTKIDINILIVLMGIIAIIYSYVGGIKSVLWTDFIQGMVLSLGAVFVVIFLCFTVKGGFSEIVSMGIKNNKFLDLSSMMDINIFRESFFITLIGAGFGTLSSYVSSQDMVQRYTTTTNIKEMKKMTYLNGVLSIGVATLFFFIGTGLYAFYTQNPSLLLTHKEDQIFASYIVSQLPAGLSGLLLAGIFAAGQSTLSSGLNSVATSWTLDVHKVIKGSMDNDKATNFAKFLSLAIGIVSIVVSIILAHSNLNSAFAWFNGFIGMVLGLVGGLFGLGVFSKKANSKGALLGFSVAVIVSIGIKYYTKVNFWAYSIISIAVCMIFGYIFSLIFKERVKENINELTIYGISKNDLNSEENIKL

SEQ ID NO: 34 - protein, 503 aa, Clostridium thermobutyricum
MQGFTKIDLVVLVAYLGAVLYAGLKFSKKEMKGKEFFKGDGTIPWWVTSVSIFATLLSPISFLSLAGNSYKGTWIMWFAQLGMFVAVPLTIKFFLPVYSKLDIDTAYEYLEIRYKDKGLRILGAIMFIIYQIGRMAIIMYLPSMVLSNLTGINVYLLIAVMGIIAIIYSYTGGLKSVLWTDFIQGCVLIAGIVFALVFLISTINGGFGSIMSELTSGGKFLSPQEPIFDANIFKTSVFLLIVGGGLNTFSSYISSQDIVQRFTTTTDIKQLKKMTYGNLVLSLFVATVFYLIGTALFVFYKQNPELVQTLQQDQIFASYIAFQLPVGITGIVLAAIYAAAQSTLSTGLNSVATSWTLDIQNFITKDMSMEKQTKIAQYISLGVGIASILMASILAGGGVNSAYEWFNGFMGLVLGVLAGIFVLGVFFKKATKAGAYAGFIASAIAVIVCKYGGFDVSIWSYSIISIGVSIIVGMIVSLMTQSQLPDEKTLNEATINLNKKVNV

SEQ ID NO: 35 - protein, 505 aa, Clostridium thermobutyricum
MKSYSPINYLVIIIYLIAVFLIGLGFSRKKQGGKVFFKGNDGSIPWWVAAVSLFATLMSPISFLTLAGRSFMTNWEDWLGQLGVFIAVPLTIMWFLPVYKRMKLDTAYEYLERRFNKKLRFLGSSMFIIFQIGRNAIIMYLPAIALAYITHMNINILILIFGVIAVIYSTLGGLKAVLWTDFIQGLVLSVGAIFVVCYLIFSVKGGFSHIVSYGLSHHKFLSTSQMMSLSIFKQGLPVVIVGAGLSTLSSYVSSQDMVQRYITTDDLGKMKKMTYLNGFMSIAIATLFFFVGTGMFVFYKQNPNLLIHAHATEDKIFASFIVNQLPVGISGLLIAALFAIGQSSLSSGINGIASTWTLDLTKSFKKNFSDEQATRYGKILSLLVGLFSIVLAIIFANSHIESIFAFFNGFVGIILGLVGGLFVLGILTKKANTKGAIIGFIVGLIISIFVNYFTSVTFWAYSIISILSVLIIGYIASFFFKDEKGSEREDLDHLTIYGSLKKDNK

SEQ ID NO: 36 - protein, 504 aa, Romboutsia ilealis
MNNMVGFTKIDFAILVLYLVAVLFAGLFFSKKEMKGKEFFKGDGTIPWWVTSVSIFATLLSPISFLSLAGNSYGGTWILWFAQLGMFIAVPLTIKFFLPLYSRLDLDTAYQYLEIRFDSKGLRILGALMFIIYQIGRMSIIMYLPSVVLAELTGISVNILIIVMGIIAIIYSYTGGLKSVLWTDFIQGVVLIVGVIGTLFFLIANINGGVGTIMETLTSGQKFISSNEVIFDINILKSSVFIIFVGAGLNTFSSYVSSQDIVQRFTTTTNLKELKKMTYGNGLLSITVATVFYLIGTCLFIFYTQNPELAQTAKQDQIFASYIAYQLPVGITGILLAAIYAASQSTLSTGLNSVATSWTLDIQTMISKDMSFERQTKIAQYISLGVGIVAIGVSVVLANGEIKSAYEWFNSFMGLVLGVLAGIFVLGAFCKSATKFGAYIGFVVSAILVVYLKYNVPDVTSWSYSLITITTSVIVGQIVSMIQLHITKRKNNVREDSTIYYEVN

SEQ ID NO: 37 - protein, 501 aa, Megamonas funiformis YIT 11815
MQGFTWIDTAVLVIYLLGVLFAGLWFSNKEHQGKEFFKGDGTIPWWVTCVSIFATLLSPISFLSLPGNSYGGSWILWWAQIGMLIAIPLTIKYFLPIYSTLQIDTAYHYLEKRFGSKGLRILGALMFIVFQTGRMSIIMYLPCIALAELTGINVNILIIVMGIIAIIYSYSGGLKAVLWTDFIQGVVLIVGIALTLVVMIASINGGFGTVMDTLTTGHKFLLENEVWFSSDILSTSVFVILVGGGLTTFSSYVSSQDIVQRFTTTTDIKQLNKMTLGNGVLSIFAATVFYLVGTSLYVFYTQNPDLLMTTRQDLVFATYITYELPAGITGILLAAIFAASQSTLSTGINSVATSWVLDIQSVINPEMSSERQTKIAQYISLGIGILAIIVAIFLASGDIKSAYEWFNSFIGLALGALVGMFVLGAFSKHANAKGAVAAFIVSSIIVIYLKYFVPSVSYWTYTIITIISSLVVGNIVSYLTDKSFVAPKGTTVYTSKAPSSK

SEQ ID NO: 38 - protein, 497 aa, Lysinibacillus sphaericus
MDGKFAVIDYVILVAYLLLILYIGVAVAKKEMKGKEFFKGDGSIPWWVTSVSLFATLLSPISFLSLAGNSYLGNWQLWAGQIGLFIAVPVAIYFFLPVYRNLNIDTAYEYLERRFGKGLRIMGSSLFIVYQVGRMSIIMYLPALALSAVTGIHTVAIILFMGVIATIYSSIGGIKSVLWTDFIQGIVLIGGGVFALIVLILSVKGGFPEIMKVGVQDNKFFSEAVIFDPNIFNDSLFIIFLGAGLSTLFSYISSQDMVQRYLTTTDLKQMNKMTIMNGVLSLGTATLFFFIGTSLYVFYKQAGGDMPDGAKDLIFANFIVTELPAGISGLLIAGLFAAGQSTLSTGLNSVATSWTLDIQKVLRPDMTDEKGTKIAKVVSIIVGVLSIVFAIVLAYSNVSSAYEWFNGLMGLVLGIIGGTFTLGVLTKRANTKGAIAGFIVTSLIAIYVSYFTDTTLWAYSLINLVSSLVFGYLFSLLFKEKSKAEDSSAGLTYYDQK

SEQ ID NO: 39 - protein, 498 aa, uncultured Clostridium sp.
MGFTTIDFIILVAYLAAVLFAGLHFSKKDMEGKEFFKGDGTIPWWVTSVSIFATLLSPISFLSLAGNSYGGTWILWFAQLGMLIAIPLTIKFFLPIYSKLNIDTAYHYLELRFGSKALRVLGALMFIVYQIGRMSIIMYLPSMVLANLTGINVNLLIIVMGVIAIIYSYTGGLKSVLWTDFIQGSVLLVGVTFALIYLLANIDGGAGAVLETLRQGKFIGAEEVIFDANILKTSVFLIVVGAGFNTCSSYISSQDIVQRFTTTTDTKQLNKMMLTNGALSIFIATVFYLIGTGLYVFYGQNPVLAQTAQQDQIFASYIAYQLPVGVTGILLAAIYAASQSTLSTGLNSVATSWTLDIQERLSKKQMSFKQQTRIAQYVSLLVGVVAIVVSMVLANGEIKSAYEWFNGFMGLVLGVLAGTFVLGVFTKVANSTGAFAAFIVSSIAMIVIKYTMPEVSIWSYSIISIGISLIVGIPVSYASRKIKNDVSVPDKDTVIYES

SEQ ID NO: 40 - protein, 499 aa, Clostridium hiranonis
MVGFTMIDLVVLVVYLLAVLFAGLFFSKKEMKGKEFFKGDGTIPWWVTSVSIFATLLSPISFLSLAGNSYAGSWILWFAQLGMFIAIPLTIRYFLPLYSRLEIDTAYQYLEMRYESKGLRILGALMFIIYQIGRMSIIMYLPSVVLSTLTGINVNVLIVVMGIIAIIYSYTGGLKAVLWTDFIQGMVLIIGVSGALIYMISQINGGFGEVMSTLTTGNKFLLPDEKLFDPNFLHTSAFIIFFGAGLNTLSSYVSSQDVVQRFTTTTDIKQLNKMTYGNGILSMGLATIFYLIGTCLFVFYGQNPELAQTVQQDQIFASYIAYELPVGVTGILLAAIYAASQSTLSTGLNSVATSWVLDIQALLSKEEIPMERQTKIAQYVSLGVGIFAIIVAIVLASGNIKSAYEWFNSFMGLVLGVLAGVFILGAFSKKADSKGAYAGFIASAIVVVYLKYFVPEVTYWAYSLITIAICVVVGLIVSKFTNKDGKEAKSETTVFYGKN

SEQ ID NO: 41 - protein, 495 aa, Clostridium niameyense
MKASFEFLDYIVLVAYLLFVLYVGVRVAKKEMKGKEYFKGDGTIPWWVSAVSLFATMLSPISYLALAGRSFKTDWAAWVAQLGVFIAVPLTIRFFLPVYKKLNLDTAYEYLERRFNKGLRLLGSVMFIVYQIGRMSIIMYLPSLALAMVTDIDINILIIAMGVIAIIYSYVGGIKSVLWTDFIQGTVLSLGAIFVVIYMAFTIHGGLGEVVRVGVENHKFLQLSSMMNLSIYKEGFFIVLIGAGLSTLASYVSSQDMVQRYTTTTDLGEMKKMTYLNGALSIGIATVFFFIGTGLFVFYKQNPALLVSGAEDKIFATYIVKQLPAGISGLLLAGVFAAGQSTLSTGLNSVATSWTLDIHKVIKGSMDNDSATKMAKILSLGVGIVSIIVAIILAHSDLKSAYNWFNGFMGLVLGVVGGLFALGIFTKKANSKGAIVGFIVSSILVVSVKYFTHVTFWMYSVISIGACMLFGYVFSLLFKEDERDLKNLTIYDRDK

SEQ ID NO: 42 - protein, 491 aa, Candidatus Arthromitus sp. SFB-turkey
MKGFSSIDFIVLIVYLIGVLLIGLYFSKKDMEGKEYFKGDGTIPWWVTSVSIFATLLSPISFLSLAGNSYSGTWALWFAQLGIFIAIPLTIRFFLPVFSKLKIDTAYHYLEIRYKSKGLRVIGAIIFIIYQIGRMSIIMYLPSIVLARLSNIHVNIFIILMGIIAIIYSYTGGIKSVLWTDFIQGVVLIVGVVGTLVFIGLKVNGGFPEIFNTLFEQGKFLSSNEVIFDPNILKTSIFMILVGGGLNTFSSYISSQDVVQRFTTTTDIKQLNRMTYTNGILSIVVATLFYLIGTALFVFYRQNPELLMTAQQDQIYASFIAYQLPAGVTGILLAAIYAASQSTLSTGLNSVATSWTLDIQNFITKDLSFKKQTKIAQLISLLVGIISIVVSIILASSEIKSAYEWFNGFIGAVLGVLAGIFVLGVVYKKATKLGAYVGFIVATIVVLALKYKLLNVDVSIWAYSLITIVVTVVVGIIVSLFSKEKNEYNTN

SEQ ID NO: 43 - protein, 617 aa, Suttonella ornithocola
MKAINQFEKWIGGIGFLLMFIILIMQITARYIHPLSWSEELARLIFVYVAMLGVSMGIRSQQHVFIDFITNRLSPRLQRSVFTLVQLLIFISIILLFYFGLKLYQKAARTPNDLIVTLGISVKWLYLALPIGSAIMLLRFIQAQYQNTKDKLSLIPSPIYIIASVILIALILWQPEIFKALRISQYTKLGSIAIYIVLGLWLLMMFFGVPVGWSLFIATVLYFSMTRWGVANFAAAKLVDSVNSFSLLSVPFFILTGLLMNSAGITTRIFSFAKTLFGHYTGGLGHVNVSASLIFSGMSGSALADAGGLGQMEIKAMRDAGYEDDLCGGLTAASCIIGPLVPPSIAMIIYGVIANQSIAELFLAGFIPGVILTIALMTMNYAICKKRGYPRAPKASRAERIKAFREAFWAVLTPLLIIGGIFSGIFTPTEAAAFAALYAVILGMFIYKELTFSALFANCVEALAISGVTVLMVMTVTFFGDMIAREQIAIKIANGFMAFADSPLMVLIMINLLLLFLGMFIDALALQFLVLPMLIPVAAQFNIDLIFFGVMTTLNMMIGILTPPMGMALFVVARVGKMPVSTVTKGAIPFIIPIFITLVILTIFPQIVTFLPNFIMR

SEQ ID NO: 44 - protein, 627 aa, Pasteurella multocida subsp. multocida OH4807
MKIINKLEEWIGGVMFIGIFLILLAQIVARQIFHSPFIWSEELARLLFIYVGLLGISMGIRSQQHVYIDFLTNFMPEKVRRLSNSFVQILIFVSIIIFIHLGFKVWLDSSFKMEALTAFVSDLVGSETIVSEKWMYAALPFISCLMLFRFLQAQVENYRNKLSHIPAIVFIISAVILFAILFIEPDWYKALRISNYVKFGSDAVYLTLIIWLVIMFLGTPVGWSLFIATLLYFSMTRWNIVNSASSKLTDSLNSFPLLSVPFFILTGILMNTGGITERIFNFARALLGHYRGGMGHVNIGASLIFSGMSGSALADAGGLGQLEIKAMRDAGYDDDICGGITAASCIIGPLVPPSIAMIIYGVIANQSIAKLFIAGFVPGVLVTIALMVMNYYVAKKRGYPRTPKATLEQRCQAFKKAIWAVLTPILIIGGIFSGLFTPTEAAVIAAFYSIVVGMFVYKELNFKMLFKSCIEAMAITGVTALMVMTVTFFGDMIAREQVAMKIAEVFVAVADSPTTVLVMINLLLLFLGMFIDALALQFLVLPMLIPIAVHFGIDLIFFGVMTTLNMMIGILTPPMGMALFVVARVGNMPVSTVAKGVLPFLVPIFVTLVLITIFPQIITFIPNLLMP

SEQ ID NO: 45 - protein, 614 aa, Pasteurella skyensis
MNMLKKFEEWTGGALLLIIFAILLAQIGSRLLNSPLIWSEELGRLIFVYAGLLGISIGVRKQQHIYIDFITNMMSDRVKKFSNSIIQLLILISILFFIKLGLHVWNDASFEMVSLNISEKWLFAPVPFISLLILIRFFETQKELYDANLSYIPPIFFVIFTVGVFVLLFFFPEVFNLLRISKYVRLGNDAVYVTLIVWLVIMFLGTPVGWSLFIATLIYFSMTRWGVVYSASEKLARSLDSFVLLSVPFFILTGVLMNTGGITERIFNFAKAMLGHHVGGMGHVNIGASLIFSGMSGSALADAGGLGQLEIKAMRDAGYDDDICGGITAASCIIGPLVPPSIAMIIYGVIANESIAKLFVAGFVPGILITIALMVMNYFVSKKRGYKPTPKASKQERWEAFRKAIWAILTPIIIIGGIFSGVFTPTEAAVIAALYSIIIGMFVYKELTLELLFKGCIETIAITGVTVLMVMTVTFFGDMIAREQVAMRIAEGFMAFADSQVSVLVMINLLLLFLGMFIDALALQFLVLPMLIPIAVHFGIDLVFFGVLTTLNMMIGILTPPMGMALFVVARVGNMTVSTVTKGVIPFIIPIFITLVLITIFPQIITFIPNLIMP

SEQ ID NO: 46 - protein, 615 aa, Haemophilus felis
MKIINKLEEWIGGVLFILIFLILLAQIVSRQFFDSPLSWSEELARLLFVYVGMLGISIGIRNQQHVFIDFVTNWLPDSIKRVTNTLVQLLIFVCLIGFIHFGLKLVFKAENELFSLGISEKWLYITLPIIACLMLVRFFQAQYDNFKNNTTYLPATFFIILMAIALAILYFSPDTYKLLRLSNYAKLGSNAVYVTLIFWLVIMFIGTPVGWSLMISTLLYFSMTRWNLSQYAAEKLVSSLDSFTLLSVPFFILTGILMNTGGITERIFSFARTLLGHYRGGMGHVNIGASLIFSGMSGSALADAGGLGQLEIKAMRDAGYDDDICGGITAASCIIGPLVPPSIAMIIYGVISNQSIAKLFIAGFVPGVLVTAALMAMNYFVAKKRGYPRSPKATLLERCDAFKKAIWAVLTPVIIIGGIFSGIFTPTEAAVIAAFYSIVIGMFVYRELTLVKLFQSCVEAMAITGVTVLMVMTVTFFGDMIAREQVAMKIANFFVSVADSPIMVLVMINLLLLFLGMFIDALALQFLVLPMLIPIAMHFGIDLIFFGVMTTLNMMIGILTPPMGMALFVVARVGNMPVSTVAKGVLPFLVPIFVTLVLITIFPQIITFIPNLLMP

SEQ ID NO: 47 - protein, 617 aa, Gammaproteobacteria bacterium
MQFLKKLNKFEEYLGGTLFLILFALLILQIVMPLLGSPLAWSDELARFIFVYVGLLGVSQGIKTQQHVLVDFVCDKLPSTPKKVVFSGVQVIIFTCLLLFIYFGVKLFIKAKFPLQSLGISEKWLYLALPTISVLMLIRFFQVQQQNVKAGLSLIPAWVFVGVTVVATALFFYDPNIYKVLRLSKYFHLGDHAVYVALLVWLVIMFLGTPVGWSLFIASIVYFSLTRWKLSVFDAFSLVDSTNNFILLAVPFFVLTGILMNSSGITERIFNFAKAMLGHHVGGMGHVNISASLIFSGMSGSALADAGGLGQLEIKAMRDAGYDDDLCGGITAASCIIGPLVPPSIAMIIYGVIANQSIAKLFLAGFVPGVMLTITLMMMNAYLCKKRAYPLAPKASSAERKVAFKKAFLPLLTPIIIIGGIFSGIFTPTESAVVAALYSVVLGFYYGELNLKKLFDNAVEAMAVSGVIVLMVMTVTYFGKMIAREQVAMQVAEGFMSVATTTVMVLIMINLLLLFLGMFIDALALQFLVLPMLIPIAQQFDIDLIFFGVMTTLNMMIGILTPPMGMALFVVARVGNMTVSTVTKGVIPFIIPIFLTLVLITIFPEVVTFIPNLVMGK

SEQ ID NO: 48 - protein, 615 aa, Pasteurella mairii
MKFINKLEEWLGGGLLLIIFAILVAQIVARQIFDSPIIWSEELARLLFIYAGLFGISMAIRSQQHVYIDFITNLMSERIRRAANSFVQTLIFISVVLFVHLGFKTWADASFEMVSLGISEKWLYAGLPFISILMFYRFLQAQSENYNNKFTYLPAIFYVISAVIIFTILFFQPDWFKVLRISNYMKLGSSAVYVVLAVWLIIMFLGTPVGWSLFIATLLYFAMTRWNVVNSASNKLVDSLNSFPLLSVPFFILTGILMNTGGITTKIFTFAKALLGHYTGGMGHVNIGASLIFSGMSGSALADAGGLGQLEIKAMRDAGYDDDICGGITAASCIIGPLVPPSIAMIIYGVIANESIAKLFIAGFVPGVLVTIALMMMNYFVSKKRGYPKTRKATREELCKAFKESIWAILTPILIIGGIFSGLFTPTEAAVFAAAYSMIVGKFVYKELSLQSLFKSCVETISITGVTALMVMTVTFFGDMIAREQVAMRIGEAFVAIADSPTMVLVMINLLLLFLGMFIDALALQFLVLPMLIPIAQQFGIDLVFFGVMTTLNMMIGILTPPMGMALFVVARVGNMTVSTVTKGVLPFLVPIFVTLVLITLFPQIITFIPNLLMP

SEQ ID NO: 49 - protein, 615 aa, Suttonella indologenes
MKALNKLEEWIGGSLFLLIFGILIFQIIAREILDSPLIWSEEAARLIFVYVGMLGISMGIRSQQHVQIDFISNRLPPKIKVAVFSIIQLLIFSCLLLFIHYGIVLMKRPPDQLVALGISTKYLYLGLPLIAGLMLLRFFQTQQENFANKAAWFPAWLYIALAVAFFAILFFAPEWFKYLRISDYVKFGKDSVYIVLLIWLVLMFLGMPVGWSLFITTVLFFSMTRWNTGNFAAAKLVDSVNSFSLLSVPFFILTGILMNSAGVTTRIFNFARAMLGHWTGGMGHVNIGASLIFSGMSGSALADAGGLGQLEIKAMRDEGYHDDLCGGITAASCIIGPLVPPSITMIIYGVIANVSIAKLFLAGFVPGVLLTIALMLMNAYICKKRGYRKAVKATRSERISSFKSSFWALLTPLLIIGGIFSGFFTPTEAAVIAALYSTVLGFVYRELNMAGLFKSCVEAMAMSGVTILMVMTVTFFGDMIARERVAIQVAEGFVAFADSALMVLLLINLLLLFLGMFIDALALQFLVLPMLIPIAQYYNIDLVFFGVMTTLNMMIGILTPPMGMALFVVARAGNMSVSTVTKGVLPFLIPIALTLVLITIFPSIITFLPNLIMGE

SEQ ID NO: 50 - protein, 617 aa, Fusobacterium canifelinum
MKVFNKLEEWLGGSLFIGMFVVLVMQIFSRQVFNSPLIWSEELSRLIFVYVGLLGVSMGIRSQQHIMIDFLYAKFPKSMQKIIFTIIQILILACLIFFLYFGYDLFIKKEEIDIVSLGISMKWMYLALPLITLLMLVRFYQAYSENYAQNKVYIKPIFILALIIILVLIAFIRPELFKVLKLSNYFDFGEMTIYYVLIAWLVMIFFGVPVGWSLLVACILYFALTRWKVVYFAADKLVYSLDSFSLLSVPFFILTGILMNGAGITERIFNFAKAMLGHYTGGMGHVNVAASLIFSGMSGSAIADAGGLGQLEIKAMRDEGYDDDICGGLTAASCIIGPLVPPSISMIIYGVIANQSIAKLFLAGFVPGFLTTIALMVMNYFVCKKRGYKKTAKASPKERWIAFKKSFWALLTPILIIGGIFSGIFTPTEAAVIATFYSIILGGFIYKELTVKSFFKHCVEAVAISGVTVLMIMTVTFFGDIIAREQVAMRVAEIFIKYATSPMMVLVMINLLLLFLGMFIDALALQFLVLPMLIPIAEQVGIDLVFFGVMTTLNMMIGILTPPMGMALFVVAQVGKMNVSTVTKGVFPFLLPIFITLVIITIFPQIILFLPNLIVGG

SEQ ID NO: 51 - protein, 617 aa, Fusobacterium varium
MKVFDKLEEWIGGTLFVCMFIILVMQIVARQVLGTPLMWSEELSSLLFVYVGMLGISMGIRNQQHVLIDFLCSRFSPKMQKIAFTIVQIIIFISIIFMGYLGNNLYKKKWIFELVSLKISAGWMYIALPIIAILMMVRFFQAYKENYDNKKVILHPGIFLAALVIIIGLIIYDPKIFRLFRLANYYKLGPIAGYVTIVVWLVMIFMGVPVGWSLMAATIFYFSITKWNVIYFASAKLVDSLNSFSLLSVPFFVLTGILMNGSGITERIFNFAKALLGHFTGGMGHVNVAASLIFSGMSGSAIADAGGLGQLEIKAMRDAGYDDDICGGITAASCIIGPLVPPSISMIVYGVIANQSIAKLFLAGFVPGVLTTIALMIMNYFICKKRGYKKAKKATFKEQVEAFKKSFWALITPFIIIGGIFSGLFTPTEAAVVAAAYSVFLGAFIYKELTVRSFFKHCVEAMAISGVTVLMIITVTFFGDMIAREQIAMKIAAGFMKYASSPLTVLVMINLLLLFLGMFIDALALQFLVLPMLIPVADKVGIDLVFFGVMTTLNMMIGILTPPMGMALFVVAQVGKMSVSTVTKGVLPFLIPIFITLVFITVFPGIITFLPNLIMGG

SEQ ID NO: 52 - protein, 617 aa, Fusobacterium necrophorum
MKFFNKLEEWIGGTLFVGMFIVLVLQIISRQILDDPLIWSEELARLFFVYVGMLGISMGIRTQSHVMIDFVYARLPKKLQKVIFTGIQIIIFLCISSFSYFGYLLIEKKADIELVSLGISAKWMYIALPVISILMLIRFFQAYQENWEKEKVLISPKIILGFMILFMALLIFQPSVFKIFKMTQYFKLRGNSVYVALGLWLILIFAGVPVGWSLLASSMVYFSMTKWAVAYFASSKFVDSVDSFSLLSVPFFILTGILMNGSGITERIFYFAKASLGHYTGGMGHVNVAASLIFSGMSGSAIADAGGLGQLEIKAMRDEGYDDDICGGITAASCIIGPLVPPSISMIIYGVIANQSIAKLFLAGFVPGVLTTIALMIMNYFVCKKRGYKKAKKCTPKERWEAFKKAFWALLTPIIIIGGIFSGMFTPTEAAVVAALYSVILGMFIYKELTWKGLFQHCVEAMAISGVTVLMIITVTFFGDMIAREQVAMKIAELFIQFASSPLTVLVMINLLLLFLGMFIDALALQFLVLPMLIPVAEQVGIDLVFFGVMTTLNMMIGILTPPMGMALFVVAQVGKMPVSTVTKGVLPFLLPIFITLVVITLFPQIIIFLPNLIMGA

SEQ ID NO: 53 - protein, 615 aa, Cardiobacterium hominis
MKVLNKLEEWVGGSLFLLIFVILVAQIFFRQVLHHPLIWSEEAARLLFVYVGMLGVSIGIRSQQHVLIDFITSHLPATLRVWVFSLAQLIIFSAILLLFYYGVTVFTKASDQLVSLGISNKWLYLSLPVCAVLMFFRFLQAQQENYRKGDSRIPVWVYMVLAAAVLLVAFIEPSWFNALRISEYVKFGKSSVYVALIIWLVIMFAGVPVGWSLFIAAVLFFSMTRWGTGNFAATKLVDSVNSFGLLSVPFFILTGILMNSAGITTRIFNFALAMLGHYTGGMGHVNIAASLIFSGMSGSALADAGGLGQLEIKAMRDAGYEDDLCGGITAASCIIGPLVPPSIAMIIYGVIADVSIAKLFLAGFVPGVLLTILLMVMNTIVCKKRGYGKAPKTSAAERRRAFKQAFWPLLTPILIIGGIFSGAFTPTEAAVIAALYSIILGMFVYRELTPAGLFTCCVEAMAITGVTVLMVMTVTFFGDMIARERVAMQVASGFMTFADNTLMVLLMINLLLLFLGMFIDALALQFLVLPMLIPIAQQFNIDLVFFGVMTTLNMMIGILTPPMGMALFVVARVGNMSVSTVTRGVIPFIIPIALCLLLITLFPGIVTFLPNLIMG

SEQ ID NO: 54 - protein, 616 aa, Aggregatibacter actinomycetemcomitans RhAA1
MKIFNKLEEWIGGVLFLVIFCILVLQILFRQAFHSPLIWSEELAKLLFVYVGMLGISVAIRKQEHVYIDFLTNLMPPTIKKVVNSFVQLVIFLGVIFFIHFGIKTFAGAGFPIDALGGISEKWIYASLPIISVLMLIRFFQAQADNFKDNKSYLPATFFIISAVILLGILFATPEWYKALRITEYVKFGSNAVYVALVFWLVIMFLGVPVGWSLFITTLLYFSMTRWNVVNAASEKLTMSLDSFPLLAVPFYILTGILMNTGGITTRIFDFAKTLLGHYTGGMGHVNIGASLLFSGMSGSALADAGGLGQLEIKAMRDAGYDDDVCGGITAASCIIGPLVPPSIAMIIYGVIANESIAKLFIAGFIPGVLVTIALMGMNYYIAKKRRYPKTSKTTRAHLCTSFKGAFWAILTPLLIIGGIFSGLFSPTESAVVAAFYSIIIGKFVYKELTLKMLFNSCVEAMAITGVVALMIMTVTFFGDMIAREQVAMRIADVFVAVADSPLMVLVMINALLLFLGMFIDALALQFLVLPMLIPIAMQFGIDLVFFGVMTTLNMMIGILTPPMGMALFVVARVGNMSVSTVTKGVLPFLVPIFITLVLITIFPQIITFVPNLLIP

SEQ ID NO: 55 - protein, 615 aa, Pasteurella bettyae CCUG 2042
MKIFNKLEEWIGGVIFLVIFAILIAQIAARQIFDAPLSWSEELARLLFVYISMLGISMAIRSQQHVFIDFITNLFPTKLKKVTNSFVQILIFISILMLAHFGYKNFVENSFEIVSLGISEKWLYASLPFISILMFIRFIQAQSDNKRENLTYLGPVFYLVLAAVLIAIYLINPDFYKIFRITNYVKLGADSVYVVLVVWLVIMFLGTPVGWSLLIATLLYFAMTRWNVSNSASIKLVESLNSFPLLSVPFFILTGILMNSGGITERIFNFAKALLGHYTGGMGHVNIGASLIFSGMSGSALADAGGLGQLEIKAMRDNGYDDDICGGITAASCIIGPLVPPSISMIIYGVISNQSIAKLFVAGFVPGVLVTIALMIMNHYVSKRRGYARTPKATLEERCQAFKKAIWAVLTPVLIIGGIFSGLFTPTEAAVISALYSTIIGMFVYKELDLKGLFKSCVEALAITGVTVLMVMTVTFFGDMIAREQVAMRIGEMFMSVADSQLMVLVMINLLLLFLGMFIDALALQFLVLPMLIPIAVQFDIDLIFFGVMTLLNMMIGILTPPMGMALFVVARVGNMSVSTVTKGVLPFLIPIFITLVLITLFPQIITFIPNLIMP

SEQ ID NO: 56 - protein, 529 aa, Haemophilus paracuniculus
MTHSFELNESRRHFMKLLAGVGAGVAFSGSLGAFSSNALAKATAGSSIEAGIAYPISTGFDPLTSSGASSLAANLHIFEGLVDLHPATRQPYLALAAKEPEQKDEVTYHITLREGATFHDGKPVTTADVVYSFERVLDPAKASLFSQFIPFIESVKAVDDKVVEFKLKYPFALFKERLTIVKIVPKHLIEEKGQAAFDANPVGSGPYQFVSATKDDRIVFKAFEAYNGLYPAKVEKMTWFLLSDDAARVTAQESNRVQAIESVPYLDAERLKRRQTVESVQSFGLLFLMFNCEKAPFNNPKVRQALHYGLDTQKLIDIVFLGNAKAATSYVQDTHPGYTKAKAQYDFDKAKAEALLKEAGVTELSFELLATDHSWVKECAPLILESWNSLNGVKVTLKHLQSGALYGSFVDTGNYEVVIAPGDPSVFGNDLDLLLSWWYRGDVWPKKRFRWSNTPEYAEVQTALDNAVRAKSNEEAKAEWDKAINIIAEQVPLYPILHRKLPSAWNDKSLTDFQPLPTTGLSFLGVGRK

SEQ ID NO: 57 - protein, 317 aa, Haemophilus paracuniculus
MEIVLRLLLRRLMALPVMMLGVTALVFIVLQFTPGDPATVALGESATEAAKALWREQRGLNDPLIVQYFRFIGNLLQFDFGVTTPPEQPIVDMIAKAFPLTLQLTLIGVFLAAIVSFTLGVTAALYRDRLVDQIIRLISVAAVATPSFWLGILLIQWFSLNLDWFPSGGFVPFSESPSEYFSSMVLPSLALAVPVCASLIRVVRTTMVEEMDKDYVRTAIGNGVPYSTVIRHNVLRNALITPVTVLGLRVGYLLGGAVVIEQIFDLPGMGKLIFNGIVNHDLNLVQGVVLTIAFTFVVVNIIVDILYLLINPKIRSL

SEQ ID NO: 58 - protein, 651 aa, Haemophilus paracuniculus
MFRQGLAERLASGGARFKALSTSAKIALLFILFVAFVAIFAPFVATFDPLQTLRPVQAPSGEYLFGTDRLGRDIFSRMVWGARTSLFIGLGAVGCAIIFGGILGATAATAQKWGNEIIMRLMDILMAFPGIALAAVLLATFGNSVPVIIITIAVVYTPQLARVVRANVVAQWEEDYVRAERVLGGSRTYILLKHVVRNTAAPVLVFATVMVADAIVFEASLSFLGAGVQPPFPSWGNILSEGRNLVLSGFWWATTFAGLMILLTVLALNILAEGLTDALVNPKLKRTPQSKENRAEKPLATDVQEAMAESLALKRYLMQLNQRETTRNDRMQLNPNAKPILQVKNLSIRFPNRYGEIPLVDNISFTVHEGETMGLVGESGCGKSITAFSIMGLLPKTAKITGEILFTDRAGNQHDLLKSADLNRLRGHEIAMIYQDALSALNPSMRIKDQMAQLISRGGKQTAETLLEWVKLDPAKTLNRYPHELSGGQRQRVLIAMALAREPKLLIADEPTTALDVTVQAEVIKLLNELREKLGFAMVFVSHDLALVAQIAHHITVMYAGQVVEAAPTSPLLANPTHEYTRGLLGSVLSTEARAKRLYQIPGSVPSPFDFAKGDRFASRSLRPDANPDKRLKLVPTADHPKHLWASHLED

SEQ ID NO: 59 - protein, 268 aa, Haemophilus paracuniculus
MSMKFIQPNNIIDMTDIVVQFPSRDGSLFNRKKITAVNGVNFSVAAGETVGIVGESGCGKSTLASVMIGLQKPTSGLVKFNGLQMQYGSAEARKQFGRQVSVIFQDPATALNPRMRVLDILKDPLDIHNILQPHEREKRVYELLSRVGLPRSAALVEPTRLSGGQKQRVAIARALALNPKLIVADEPTSALDVSVRAQVLNLLADLKKELNLAMVFISHDIQTVRQVSDKIVVMYAGQIVESGTVEQIFENPKMDYTKRLLGAAPSLL

SEQ ID NO: 60 - protein, 299 aa, Helcobacter pylori UA1234
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 61 - protein, 223 aa, Pasteurella multocida
MTNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAARQTLPKMYRANGAIYINDIAQLLKEKYFFIPPLKFYLMPTYRSVDIDVKQDLELAEILSNK

SEQ ID NO: 62 - protein, 224 aa, Haemophilus influenzae
MTRIAIIPARAGSKGIKDKNLQLVGGVSLVGRAILAAQESGMFDQIVVTSDGENILKEATKYGAKAVARPESLAQSDTRTIDAILHCLETLNISQGTAALLQPTSPLRNALDIRNAMEIFLGGKYKSVVSACECEHHPYKSFTLEGTEVQPIHELTDFEAPRQKLPKSYRANGAIYTNDIQSLFEEKRFFIAPMRFYLMPTYRSIDIDSTLDLQLAESLISKEF

SEQ ID NO: 63 - protein, 348 aa, Syntrophorhabdus sp. PtaU1.Bin058
MESFSFGKSTVSKNGPCYIIAEIGNNHQGDLKIALKMIKTAAGMGVNAVKFQKRDNRTLYTKSMYNKPYENENSYGSTYGEHRDYLEFCWDKYVEMKKCAEDNDVEFMCTAFDFKSVDFLEQLGITSYKVASGDVTNTPLLEYIAQTKKPMFVSTGGATLEEIRVAYDTVTKYNDRVCLLHCVAGYPTEYEYLNLNTINILKKEFPDTLIGYSGHDYGILAPVVAYMLGAVVVEKHFTLNRAWKGTDHKFSLEPTGLYKMTRDLRRVDVSIGNGKKVVHAFEKDPISKMSKSLYASRKLQAGRVLTADDISIKSPGGGIPPYKMEEVLGKMLKINLPEEALFSFDYFE

SEQ ID NO: 64 - protein, 308 aa, Pasteurella multocida subsp. multocida str. Pm70
MNLIICCTPLQVLIAEKIIAKFPHMPFYGVMLSTVSNKKFDFYAKRLAQQCQGFFSMVQHKDRFNLLKEILYLKRTFSGKHFDQVFVANINDLQIQFLLSAIDFNLLNTFDDGTINIVPNSLFYQDDPATLQRKLINVLLGNKYSIQSLRALSHTHYTIYKGFKNIIERVEPIELVAADNSEKVTSAVINVLLGQPVFAEDERNIALAERVIKQFNIHYYLPHPREKYRLAQVNYIDTELIFEDYILQQCQTHKYCVYTYFSSAIINIMNKSDNIEVVALKIDTENPAYDACYDLFDELGVNVIDIRE

SEQ ID NO: 65 - protein, 371 aa, Neisseria meningitidis
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 66 - protein, 348 aa, Neisseria meningitidis 35E
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

(19) United States
(12) Patent Application Publication
(10) Pub. No.: US 2024/0200021 A1
(43) Pub. Date: Jun. 20, 2024

(54) CELLULAR PRODUCTION OF SIALYLATED DI- AND/OR OLIGOSACCHARIDES

(71) Applicant: INBIOSE N.V., Zwijnaarde (BE)

(72) Inventors: Joeri Beauprez, Zwijnaarde (BE); Pieter Coussement, Zwijnaarde (BE); Thomas Decoene, Zwijnaarde (BE)

(21) Appl. No.: 18/555,663

(22) PCT Filed: Apr. 15, 2022
(86) PCT No.: PCT/EP2022/060181

§ 371 Date: Oct. 16, 2023

(30) Foreign Application Priority Data
Apr. 16, 2021 (EP) 21168996.3

(51) Int. Cl. C12N 1/20; C12N 1/18; C12N 9/10; C12P 9/00; C12P 19/04; C12P 19/12; C12P 19/26; C12R 1/19

(52) U.S. Cl. CPC C12N 1/205; C12N 1/185; C12N 9/1081; C12P 9/00; C12P 19/04; C12P 19/12; C12P 19/26; C12N 2500/34; C12R 2001/19

(57) ABSTRACT

The present invention is in the technical field of synthetic biology and metabolic engineering. More particularly, the present invention is in the technical field of metabolically engineered cells of microorganisms and the use of said cells in a fermentation. The present invention describes a metabolically engineered cell of a microorganism and a method by fermentation with said cell for production of a sialylated di- and/or oligosaccharide. The metabolically engineered cell comprises a pathway for production of said sialylated di- and/or oligosaccharide, synthesizes sialic acid, expresses at least one sialyltransferase, preferably is modified in the expression or activity of at least one sialyltransferase, is modified to have a fully or partially knocked out or rendered less functional sialic acid catabolic pathway and is modified for overexpression of an endogenous sialic acid transporter and/or expression, preferably overexpression, of an exogenous, homologous and/or heterologous sialic acid transporter. Furthermore, the present invention provides for purification of said sialylated di- and/or oligosaccharide from the cultivation, preferably fermentation.

### FIELD OF THE INVENTION

The present invention is in the technical field of synthetic biology and metabolic engineering. More particularly, the present invention is in the technical field of metabolically engineered cells of microorganisms and the use of said cells in a fermentation. The present invention describes a metabolically engineered cell of a microorganism and a method by fermentation with said cell for production of a sialylated di- and/or oligosaccharide. The metabolically engineered cell comprises a pathway for production of said sialylated di- and/or oligosaccharide, synthesizes sialic acid, expresses at least one sialyltransferase, preferably is modified in the expression or activity of at least one sialyltransferase, is modified to have a fully or partially knocked out or rendered less functional sialic acid catabolic pathway and is modified for overexpression of an endogenous sialic acid transporter and/or expression, preferably overexpression, of an exogenous, homologous and/or heterologous sialic acid transporter. Furthermore, the present invention provides for purification of said sialylated di- and/or oligosaccharide from the cultivation, preferably fermentation.

### BACKGROUND

Sialylated di- and oligosaccharides, often present as glyco-conjugated forms to proteins and lipids, are involved in many vital phenomena such as development, differentiation, fertilization, embryogenesis, host pathogen adhesion and inflammation. Sialylated oligosaccharides can also be present as unconjugated glycans in body fluids and mammalian milk wherein they modulate as bioactive glycans in important developmental and immunological processes (Bode, Early Hum. Dev. 2015, 91(11): 619-622; Bode, Nestle Nutr. Inst. Workshop Ser. 2019, 90: 191-201; Reily et al., Nat. Rev. Nephrol. 2019, 15: 346-366; Varki, Glycobiology 2017, 27: 3-49; Walsh et al., J. Funct. Foods 2020, 72: 10474). There is large scientific and commercial interest in sialylated di- and oligosaccharides due to their wide functional spectrum. Yet, the availability of sialylated di- and/or oligosaccharides is limited as production relies on chemical or chemo-enzymatic synthesis or on purification from natural sources such as e.g. animal milk. Chemical synthesis methods are laborious and time-consuming and because of the large number of steps involved they are difficult to scale-up. Enzymatic approaches using nucleotide-activated sugars and glycosyltransferases offer many advantages above chemical synthesis. Glycosyltransferases catalyse the transfer of a sugar moiety from a nucleotide-activated sugar donor onto saccharide or non-saccharide acceptors (Coutinho et al., J. Mol. Biol. 2003, 328: 307-317). These glycosyltransferases are the source for biotechnologists to synthesize sialylated di- and oligosaccharides and are used both in (chemo)enzymatic approaches as well as in cell-based production systems. However, stereospecificity and regioselectivity of glycosyltransferases are still a formidable challenge. In addition, chemo-enzymatic approaches need to regenerate in situ nucleotide-activated sugar donors. Cellular production of sialylated di- and oligosaccharides needs tight control of spatiotemporal availability of adequate levels of nucleotide-activated sugar donors in proximity of complementary glycosyltransferases. Due to these difficulties, current methods often result in small-scale synthesis of sialylated di- and/or oligosaccharides.

PEP or phosphoenolpyruvate is a common precursor in the anabolism of a cell and of key importance for the synthesis of secondary metabolites such as flavonoids, aromatic amino acids and many monosaccharide subunits of sialylated di- and oligosaccharides or sialylated di- and oligosaccharide modifications. Such monosaccharide subunits are for instance N-acetylneuraminic acid, legionaminic acid, ketodeoxyoctonate, keto-deoxy-nonulonic acid, pseudaminic acid, N,N′-diacetyl-8-epilegionaminate, N-acetyl-D-muramate and their nucleotide and phosphorylated derivatives. To enhance synthesis of these monosaccharide subunits and sialylated di- and/or oligosaccharides, the PEP concentration in the cell can be enhanced by means of overexpression and deletion of several genes.

Zhu et al. (Biotechnol. Lett. 2017, 39: 227-234) has shown that by the overexpression of PEP synthase (EC: 2.7.9.2) and PEP carboxykinase (EC: 4.1.1.49) the synthesis of N-acetylneuraminic acid was increased by 96.4% and 61% compared to the control respectively, combined overexpression increased the synthesis further up to 116.7% compared to the control. Zhu et al. (Biotechnol. Lett 2016, doi 10.1007/s10529-016-2215-z) has further shown that the deletion of a substrate phosphotransferase (PTS) system like the N-acetylglucosamine PTS system encoded by the gene nagE in E. coli, transporting and phosphorylating with the use of PEP N-acetylglucosamine (GlcNAc) and glucosamine (GlcN) into the cell, or like the mannose PTS system encoded by the genes manX, manYand manZ in E. coli, transporting and phosphorylating with the use of PEP mannose, N-acetylmannosamine, glucose, fructose, GlcN and GlcNAc into the cell, increases Neu5Ac synthesis significantly. The upregulation of ppsA in E. coli was later also shown to be effective in EP3697805 and EP3575404, combining also ppsA overexpression with the deletion of manXYZ and nagE.

Zhang et al. (Biotech and Bioeng. 2018, 115(9): 217-2231) improved PEP synthesis in Bacillus subtilis in a similar fashion. The glucose PTS system was deleted to reduce PEP usages upon glucose uptake, the gene pyruvate kinase (EC: 2.7.1.40) was deleted to reduce PEP consumption and the gene PEP carboxykinase (EC: 4.1.1.49) was overexpressed to enhance the flux towards. To compensate for the deletion of the glucose PTS system, glucose permease and glucokinase were used to internalize and phosphorylate glucose in the cell. Further, the malic enzyme (EC: 1.1.1.38, EC: 1.1.1.39 or EC: 1.1.1.40) was introduced to increase the flux from the Krebs cycle towards pyruvate, the precursor of PEP. A reduced glycolysis and the introduction of the Entner-Doudoroff pathway further enhanced the production of N-acetylneuraminate. Note that these strains are in their basis modified in their acetate and lactate synthesis capacity, which inherently leads to improved availability of PEP, pyruvate and acetyl-CoA.

Zhang et al. (Biotech. Adv. 2019, 37: 787-800) also reviewed and described how the precursors of N-acetylneuraminic acid and sialylated oligosaccharides can be modulated. By impacting the PEP and pyruvate availability in the cell, the flux towards sialylated oligosaccharides and N-acetylneuraminate (or other monosaccharide subunits as described above) is enhanced. Also here, techniques are described to delete or knock down the glycolysis pathway (comprising phosphofructokinase (pfkA gene, E.C.:2.7.1.11) and pyruvate kinase (pyk, EC: 2.7.1.40)) and to upregulate the phosphoenolpyruvate synthase gene (ppsA, EC: 2.7.9.2). Introduction or overexpression of the Entner-Doudoroff pathway and reduced PTS activity further led to improvements in synthesis. The system described was not only achieved by overexpression or deletions, but also by dynamic control through biosensors, which selectively upregulate and downregulate reactions in the cellular biochemistry.

It is an object of the present invention to provide for tools and methods by means of which a sialylated di- and/or oligosaccharide can be produced by a cell of a microorganism and preferably in an efficient, time and cost-effective way and which yields high amounts of the desired sialylated di- and/or oligosaccharide.

According to the invention, this and other objects are achieved by providing a cell of a microorganism and a method for the production of a sialylated di- and/or oligosaccharide wherein the cell is metabolically engineered for the production of said sialylated di- and/or oligosaccharide and wherein the cell synthesizes sialic acid, expresses at least one sialyltransferase, preferably is modified in the expression or activity of at least one sialyltransferase, is modified to have a fully or partially knocked out or rendered less functional sialic acid catabolic pathway and is modified for overexpression of an endogenous sialic acid transporter and/or expression, preferably overexpression, of an exogenous, homologous and/or heterologous sialic acid transporter as defined herein.

### DESCRIPTION

### Summary of the Invention

It has now been found that a cell of a microorganism comprising a pathway for the production of a sialylated di- and/or oligosaccharide, wherein the cell synthesizes sialic acid, expresses at least one sialyltransferase, preferably is modified in the expression or activity of at least one sialyltransferase, is modified to have a fully or partially knocked out or rendered less functional sialic acid catabolic pathway and is modified for (over)expression of at least one sialic acid transporter provides for a cell which, when used in a fermentative production and cultivating the cell under conditions permissive to produce said sialylated di- and/or oligosaccharide, produces more of a sialylated di- and/or oligosaccharide when compared to a corresponding cell but without the extra modification in the (over)expression of said sialic acid transporter. The present invention also provides a method for the production of a sialylated di- and/or oligosaccharide using the cell of the invention. The present invention also provides methods to separate said sialylated di- and/or oligosaccharide. Furthermore, is has been found that the sialic acid transporters identified in the present invention provide for enzymes enabling fermentative production of a sialylated di- and/or oligosaccharide, and preferably having a positive effect, and even more preferably providing a better yield, productivity and/or specific productivity when used to metabolically engineer a cell of a microorganism producing said sialylated di- and/or oligosaccharide when compared to a cell with the same genetic background but lacking the sialic acid transporter identified in the present invention.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of the invention disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims which follow, reference characters used to designate claim steps are provided for convenience of description only, and are not intended to imply any particular order for performing the steps.

In this document and in its claims, the verb “to comprise” and its conjugations is used in its non-limiting sense to mean that items following the word are included, but items not specifically mentioned are not excluded. Throughout the application, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”. Throughout the application, unless explicitly stated otherwise, the articles “a” and “an” are preferably replaced by “at least two”, more preferably by “at least three”, even more preferably by “at least four”, even more preferably by “at least five”, even more preferably by “at least six”, most preferably by “at least two”.

Throughout the application, unless explicitly stated otherwise, the features “synthesize”, “synthesized” and “synthesis” are interchangeably used with the features “produce”, “produced” and “production”, respectively.

Throughout the application, unless explicitly stated otherwise, the expressions “capable of . . . <verb>” and “capable to . . . <verb>” are preferably replaced with the active voice of said verb and vice versa. For example, the expression “capable of expressing” is preferably replaced with “expresses” and vice versa, i.e. “expresses” is preferably replaced with “capable of expressing”.

Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference.

According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

The terms “recombinant” or “transgenic” or “engineered” or “metabolically engineered” or “genetically engineered” or “genetically modified” as used herein with reference to a cell or host cell are used interchangeably and indicates that the cell replicates a heterologous nucleic acid, or expresses a peptide or protein encoded by a heterologous nucleic acid (i.e., a sequence “foreign to said cell” or a sequence “foreign to said location or environment in said cell”). Such cells are described to be transformed with at least one heterologous or exogenous gene, or are described to be transformed by the introduction of at least one heterologous or exogenous gene. Metabolically engineered or recombinant or transgenic or genetically engineered cells can contain genes that are not found within the native (non-recombinant) form of the cell. Recombinant cells can also contain genes found in the native form of the cell wherein the genes are re-introduced into the cell by artificial means. The native genes of the cell can also be modified before they are re-introduced into the recombinant cells.

The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one which has been produced by a recombinant cell.

The terms also encompass cells that have been modified by removing a nucleic acid endogenous to the cell by means of common well-known technologies for a skilled person (like e.g. knocking-out genes).

A “heterologous sequence” or a “heterologous nucleic acid”, as used herein, is one that originates from a source foreign to the particular cell (e.g. from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g. by transfection, transformation, conjugation or transduction, into the genome of the host microorganism cell, wherein techniques may be applied which will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell or microorganism as used within the context of the present disclosure refers to a cell or microorganism which is genetically engineered.

The term “endogenous,” within the context of the present disclosure refers to any polynucleotide, polypeptide or protein sequence which is a natural part of a cell and is occurring at its natural location in the cell chromosome. The term “exogenous” refers to any polynucleotide, polypeptide or protein sequence which originates from outside the cell under study and not a natural part of the cell or which is originating from the cell but not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism. In contrast a “homologous” polynucleotide, gene, nucleic acid, polypeptide, or enzyme is used herein to denote a polynucleotide, gene, nucleic acid, polypeptide, or enzyme that is derived from the host organism. When referring to a gene regulatory sequence or to an auxiliary nucleic acid sequence used for maintaining or manipulating a gene sequence (e.g. a promoter, a 5 untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter”, even though the promoter may be derived from the same species (or, in some cases, the same organism) as the gene to which it is linked.

The term “polynucleotide encoding a polypeptide” as used herein encompasses polynucleotides that include a sequence encoding a polypeptide of the invention. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild type expression of said gene in any phase of the production process of the desired sialylated di- and/or oligosaccharide. Said modified expression is either a lower or higher expression compared to the wild type, wherein the terms “higher expression” or “enhanced expression” are also defined as “overexpression” of said gene in the case of an endogenous gene or “expression” in the case of a heterologous gene that is not present in the wild type strain. Lower or reduced expression is obtained by means of common well-known technologies for a skilled person (such as the usage of siRNA, CrispR, CrispRi, riboswitches, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e. statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can for instance be obtained by mutating one or more base pairs in the promoter sequence or changing the promoter sequence fully to a constitutive promoter with a lower expression strength compared to the wild type or an inducible promoter which result in regulated expression or a repressible promoter which results in regulated expression. Overexpression or expression is obtained by means of common well-known technologies for a skilled person (such as the usage of artificial transcription factors, de novo design of a promoter sequence, ribosome engineering, introduction or re introduction of an expression module at euchromatin, usage of high-copy-number plasmids), wherein said gene is part of an “expression cassette” which relates to any sequence in which a promoter sequence, untranslated region sequence (containing either a ribosome binding sequence or Kozak sequence or Shine Dalgarno sequence), a coding sequence (for instance a sialic acid transporter) and optionally a transcription terminator is present, and leading to the expression of a functional active protein. Said expression is either constitutive or conditional or regulated or tuneable.

The term “constitutive expression” is defined as expression that is not regulated by transcription factors other than the subunits of RNA polymerase (e.g. the bacterial sigma factors like s⁷⁰, s⁵⁴, or related s-factors and the yeast mitochondrial RNA polymerase specificity factor MTF1 that co-associate with the RNA polymerase core enzyme) under certain growth conditions. Non-limiting examples of such transcription factors are CRP, LacI, ArcA, Cra, IcIR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. The RNA polymerase is the catalytic machinery for the synthesis of RNA from a DNA template. RNA polymerase binds a specific DNA sequence to initiate transcription, for instance via a sigma factor in prokaryotic hosts or via MTF1 in yeasts. Constitutive expression offers a constant level of expression with no need for induction or repression.

The term “regulated expression” is defined as a facultative or regulatory or tuneable expression of a gene that is only expressed upon a certain natural condition of the host (e.g. mating phase of budding yeast, stationary phase of bacteria), as a response to an inducer or repressor such as but not limited to glucose, allo-lactose, lactose, galactose, glycerol, arabinose, rhamnose, fucose, IPTG, methanol, ethanol, acetate, formate, aluminium, copper, zinc, nitrogen, phosphates, xylene, carbon or nitrogen depletion, or substrates or the produced product or chemical repression, as a response to an environmental change (e.g. anaerobic or aerobic growth, oxidative stress, pH shifts, temperature changes like e.g. heat-shock or cold-shock, osmolarity, light conditions, starvation) or dependent on the position of the developmental stage or the cell cycle of said host cell including but not limited to apoptosis and autophagy. Regulated expression allows for control as to when a gene is expressed.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous. The term “wild type” refers to the commonly known genetic or phenotypical situation as it occurs in nature.

The term “modified expression or activity of a protein” as used herein refers to i) higher expression or overexpression of an endogenous protein, ii) lower, reduced and/or abolished expression of an endogenous protein, iii) expression of an exogenous, heterologous or homologous protein, or iv) expression and/or overexpression of a mutant protein that has a higher, accelerated or lower activity compared to the wild-type (i.e. native) protein.

The term “modified expression or activity of a sialyltransferase” as used herein refers to i) higher expression or overexpression of an endogenous sialyltransferase, ii) expression of a heterologous sialyltransferase and/or iii) expression and/or overexpression of a mutant sialyltransferase that has a higher sialyltransferase activity compared to the wild-type (i.e. native) sialyltransferase protein.

Expression of a gene may be enhanced by, as described in WO 00/18935, WO98/04715, substituting an expression regulatory sequence such as the native promoter with a stronger promoter, whether the gene is present on the chromosome or a plasmid, amplifying a regulatory element that is able to increase expression of the gene, or deleting or attenuating a regulatory element that decreases expression of the gene. Examples of known strong promoters include the lac promoter, trp promoter, trc promoter, tac promoter, lambda phage PR promoter, PL promoter, and tet promoter. A method to evaluate the strength of a promoter and examples of strong promoters are described in Goldstein et al. (Prokaryotic promoters in biotechnology. Biotechnol. Annu. Rev., 1995, 1, 105-128) or the like. In addition, it is known that a spacer sequence between the ribosome binding site (RBS) and the translation initiation codon, especially, several nucleotides just upstream of the initiation codon, has a great influence on translation efficiency. Therefore, this sequence may be modified. In addition, to enhance the activity of a protein encoded by a gene, a mutation that increases the activity may be introduced into said gene. Examples of such a mutation include a mutation in the promoter sequence to increase the transcription level of said gene, and a mutation in the coding region to increase the specific activities of the protein.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

The term “derivative” of a polypeptide, as used herein, is a polypeptide which may contain deletions, additions or substitutions of amino acid residues within the amino acid sequence of the polypeptide, but which result in a silent change, thus producing a functionally equivalent polypeptide. Amino acid substitutions may be made based on similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; planar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Within the context of this invention, a derivative polypeptide as used herein, refers to a polypeptide capable of exhibiting a substantially similar in vitro and/or in vivo activity as the original polypeptide as judged by any of a number of criteria, including but not limited to enzymatic activity, and which may be differentially modified during or after translation. Furthermore, non-classical amino acids or chemical amino acid analogues can be introduced as a substitution or addition into the original polypeptide sequence.

In some embodiments, the present disclosure contemplates making functional variants by modifying the structure of a sialic acid transporter as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, and in some cases to provide better yield, productivity, and/or growth speed than a cell without the variant.

The term “functional homolog” as used herein describes those molecules that have sequence similarity and also share at least one functional characteristic such as a biochemical activity. More specifically, the term “functional homolog” as used herein describes those proteins that have sequence similarity (in other words, homology) and at the same time have at least one functional similarity such as a biochemical activity (Altenhoff et al., PLoS Comput. Biol. 8 (2012) e1002514).

Functional homologs are sometimes referred to as orthologs, where “ortholog” refers to a homologous gene or protein that is the functional equivalent of the referenced gene or protein in another species. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events.

Functional homologs can be identified by analysis of nucleotide and polypeptide sequence alignments. For example, performing a query on a database of nucleotide or polypeptide sequences can identify homologs of the nucleotide or polypeptide of interest. Sequence analysis can involve BLAST, Reciprocal BLAST, or PSI-BLAST analysis of non-redundant databases using the nucleotide or the amino acid sequence of a reference nucleotide or polypeptide sequence. The amino acid sequence is, in some instances, deduced from the nucleotide sequence. Typically, those polypeptides in the database that have greater than 40 percent sequence identity to a polypeptide of interest are candidates for further evaluation for suitability as a homologous polypeptide. Amino acid sequence similarity allows for conservative amino acid substitutions, such as substitution of one hydrophobic residue for another or substitution of one polar residue for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out in order to narrow the number of candidates to be further evaluated.

A domain can be characterized, for example, by a Pfam (El-Gebali et al., Nucleic Acids Res. 47 (2019) D427-D432), an IPR (InterPro domain) (http://ebi.ac.uk/interpro) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (https://www.ncbi.nlm.nih.gov/cdd) (Lu et al., Nucleic Acids Res. 48 (2020) D265-D268), a PTHR domain (http://www.pantherdb.org) (Mi et al., Nucleic Acids. Res. 41 (2013) D377-D386; Thomas et al., Genome Research 13 (2003) 2129-2141) or a PATRIC identifier or PATRIC DB global family domain (https://www.patricbrc.org/) (Davis et al., Nucleic Acids Res. 48(D1) (2020) D606-D612). It should be understood for those skilled in the art that for the databases used herein, comprising Pfam 32.0 (released September 2018), CDD v3.17 (released 3 Apr. 2019), eggnogdb 5.0.0 (released November 2018), InterPro 75.0 (released 4 Jul. 2019), TCDB (released 17 Jun. 2019) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

Protein or polypeptide sequence information and functional information can be provided by a comprehensive resource for protein sequence and annotation data like e.g. the Universal Protein Resource (UniProt) (www.uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers are unique for each protein present in the database and are defined herein as “UniProt ID” or “UniProtKB ID” or “UniProtKB” or “UniProt KB”. The UniProt identifiers as used herein are the UniProt identifiers in the UniProt database version release 2021_02 of 7 Apr. 2021. Proteins that do not have an UniProt ID are referred herein using the respective GenBank Accession number (GenBank No.) as present in the NIH genetic sequence database (https://www.ncbi.nlm.nih.gov/genbank/) (Nucleic Acids Res. 2013, 41(D1), D36-D42) under GenBank Release 236.0 of 15 Feb. 2020. Throughout the application, the sequence of a polypeptide can be represented by a SEQ ID NO or alternatively by an UniProt ID. Therefore, the terms “polypeptide SEQ ID NO” and “polypeptide UniProt ID” can be interchangeably used, unless explicitly stated otherwise.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. The percentage of sequence identity can be, preferably is, determined by alignment of the two sequences and identification of the number of positions with identical residues divided by the number of residues in the shorter of the sequences×100. Percent identity may be calculated globally over the full-length sequence of the reference sequence, resulting in a global percent identity score. Alternatively, percent identity may be calculated over a partial sequence of the reference sequence, resulting in a local percent identity score. A partial sequence preferably means at least about 50%, 60%, 70%, 80%, 90% or 95% of the full-length reference sequence. In another preferred embodiment, a partial sequence of a reference polypeptide sequence means a stretch of at least 200 amino acid residues up to the total number of amino acid residues of a reference polypeptide sequence. Using the full-length of the reference sequence in a local sequence alignment results in a global percent identity score between the test and the reference sequence. Percent identity can be determined using different algorithms like for example BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

The terms “sialic acid”, “N-acetylneuraminate”, “N-acylneuraminate”, “N-acetylneuraminic acid”, “Neu(n)Ac”, “NeuAc” are used interchangeably and refer to an acidic sugar with a nine-carbon backbone comprising but not limited to Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4, Neu4,5,7,8,9Ac5 and Neu5Gc. Neu4Ac is also known as 4-O-acetyl-5-amino-3,5-dideoxy-D-glycero-D-galacto-non-2-ulopyranosonic acid or 4-O-acetyl neuraminic acid and has C11H19NO9 as molecular formula. Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C11H19NO9 as molecular formula. Neu4,5Ac2 is also known as N-acetyl-4-O-acetylneuraminic acid, 4-O-acetyl-N-acetylneuraminic acid, 4-O-acetyl-N-acetylneurammate, 4-acetate 5-acetamido-3,5-dideoxy-D-glycero-D-galacto-nonulosonate, 4-acetate 5-(acetylamino)-3,5-dideoxy-D-glycero-D-galacto-2-nonulosonate, 4-acetate 5-acetamido-3,5-dideoxy-D-glycero-D-galacto-nonulosonic acid or 4-acetate 5-(acetylamino)-3,5-dideoxy-D-glycero-D-galacto-2-nonulosonic acid and has C13H21NO10 as molecular formula. Neu5,7Ac2 is also known as 7-O-acetyl-N-acetylneuraminic acid, N-acetyl-7-O-acetylneuraminic acid, 7-O-acetyl-N-acetylneuraminate, 7-acetate 5-acetamido-3,5-dideoxy-D-glycero-D-galacto-nonulosonate, 7-acetate 5-(acetylamino)-3,5-dideoxy-D-glycero-D-galacto-2-nonulosonate, 7-acetate 5-acetamido-3,5-dideoxy-D-glycero-D-galacto-nonulosonic acid or 7-acetate 5-(acetylamino)-3,5-dideoxy-D-glycero-D-galacto-2-nonulosonic acid and has C13H21NO10 as molecular formula. Neu5,8Ac2 is also known as 5-n-acetyl-8-o-acetyl neuraminic acid and has C13H21NO10 as molecular formula. Neu5,9Ac2 is also known as N-acetyl-9-O-acetylneuraminic acid, 9-anana, 9-O-acetylsialic acid, 9-O-acetyl-N-acetylneuraminic acid, 5-n-acetyl-9-O-acetyl neuraminic acid, N,9-O-diacetylneuraminate or N,9-O-diacetylneuraminate and has C13H21NO10 as molecular formula. Neu4,5,9Ac3 is also known as 5-N-acetyl-4,9-di-O-acetylneuraminic acid. Neu5,7,9Ac3 is also known as 5-N-acetyl-7,9-di-O-acetylneuraminic acid. Neu5,8,9Ac3 is also known as 5-N-acetyl-8,9-di-O-acetylneuraminic acid. Neu4,5,7,9Ac4 is also known as 5-N-acetyl-4,7,9-tri-O-acetylneuraminic acid. Neu5,7,8,9Ac4 is also known as 5-N-acetyl-7,8,9-tri-O-acetylneuraminic acid. Neu4,5,7,8,9Ac5 is also known as 5-N-acetyl-4,7,8,9-tetra-O-acetylneuraminic acid. Neu5Gc is also known as N-glycolylneuraminic acid, N-glycolylneuraminic acid, N-glycolylneuraminate, N-glycoloyl-neuraminate, N-glycoloylneuraminic acid, N-glycoloylneuraminic acid, 3,5-dideoxy-5-((hydroxyacetyl)amino)-D-glycero-D-galacto-2-nonulosonic acid, 3,5-dideoxy-5-(glycoloylamino)-D-glycero-D-galacto-2-nonulopyranosonic acid, 3,5-dideoxy-5-(glycoloylamino)-D-glycero-D-galacto-non-2-ulopyranosonic acid, 3,5-dideoxy-5-[(hydroxyacetyl)amino]-D-glycero-D-galacto-non-2-ulopyranosonic acid, D-glycero-5-glycolylamido-3,5-dideoxy-D-galacto-non-2-ulo-pyranosonic acid and has C11H19NO10 as molecular formula.

The terms “N-acetylneuraminic acid synthase”, “N-acetylneuraminate synthase”, “sialic acid synthase”, “NeuAc synthase”, “NeuB”, “NeuB1”, “NeuNAc synthase”, “Neu(n)Ac synthase”, “NANA condensing enzyme”, “N-acetylneuraminate lyase synthase”, “N-acetylneuraminic acid condensing enzyme” as used herein are used interchangeably and refer to an enzyme capable to synthesize sialic acid from N-acetylmannosamine (ManNAc) in a reaction using phosphoenolpyruvate (PEP).

The terms “CMP-sialic acid synthase”, “N-acylneuraminate cytidylyltransferase”, “CMP-sialate synthase”, “CMP-NeuAc synthase”, “CMP-Neu(n)Ac synthase”, “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

The terms “L-glutamine-D-fructose-6-phosphate aminotransferase”, “glutamine-fructose-6-phosphate transaminase (isomerizing)”, “hexosephosphate aminotransferase”, “glucosamine-6-phosphate isomerase (glutamine-forming)”, “glutamine-fructose-6-phosphate transaminase (isomerizing)”, “D-fructose-6-phosphate am idotransferase”, “fructose-6-phosphate am inotransferase”, “glucosaminephosphate isomerase”, “glucosamine 6-phosphate synthase”, “GlcN6P synthase”, “GFA”, “glms”, “glmS” and “glmS*54” are used interchangeably and refer to an enzyme that catalyses the conversion of D-fructose-6-phosphate into D-glucosamine-6-phosphate using L-glutamine.

The terms “glucosamine-6-P deaminase”, “glucosamine-6-phosphate deaminase”, “GlcN6P deaminase”, “glucosamine-6-phosphate isomerase”, “glmD” and “nagB” are used interchangeably and refer to an enzyme that catalyses the reversible isomerization-deamination of glucosamine-6-phosphate (GlcN6P) to form fructose-6-phosphate and an ammonium ion.

The terms “phosphoglucosamine mutase” and “glmM” are used interchangeably and refer to an enzyme that catalyses the conversion of glucosamine-6-phosphate to glucosamine-1-phosphate.

Phosphoglucosamine mutase can also catalyse the formation of glucose-6-P from glucose-1-P, although at a 1400-fold lower rate.

The terms “N-acetylglucosamine-6-P deacetylase”, “N-acetylglucosamine-6-phosphate deacetylase” and “nagA” are used interchangeably and refer to an enzyme that catalyses the hydrolysis of the N-acetyl group of N-acetylglucosamine-6-phosphate (GlcNAc-6-P) to yield glucosamine-6-phosphate (GlcN6P) and acetate.

An N-acylglucosamine 2-epimerase is an enzyme that catalyses the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase, N-acyl-D-glucosamine 2-epimerase and N-acetylglucosamine epimerase.

A UDP-N-acetylglucosamine 2-epimerase is an enzyme that catalyses the reaction N-acetyl-D-glucosamine=N-acetylmannosamine. Alternative names for this enzyme comprise UDP-N-acylglucosamine 2-epimerase, UDP-GlcNAc-2-epimerase, “neuC” and UDP-N-acetyl-D-glucosamine 2-epimerase.

A bifunctional UDP-GlcNAc 2-epimerase/kinase is a bifunctional enzyme that catalyses the reaction UDP-N-acetyl-D-glucosamine=N-acetyl-D-mannosamine and the reaction N-acetyl-D-mannosamine+ATP=ADP+N-acetyl-D-mannosamine 6-phosphate.

A glucosamine 6-phosphate N-acetyltransferase is an enzyme that catalyses the transfer of an acetyl group from acetyl-CoA to D-glucosamine-6-phosphate thereby generating a free CoA and N-acetyl-D-glucosamine 6-phosphate. Alternative names comprise aminodeoxyglucosephosphate acetyltransferase, D-glucosamine-6-P N-acetyltransferase, glucosamine 6-phosphate acetylase, glucosamine 6-phosphate N-acetyltransferase, glucosamine-phosphate N-acetyltransferase, glucosamine-6-phosphate acetylase, N-acetylglucosamine-6-phosphate synthase, phosphoglucosamine acetylase, phosphoglucosamine N-acetylase phosphoglucosamine N-acetylase, phosphoglucosamine transacetylase, GNA and GNA1.

The term “N-acetylglucosamine-6-phosphate phosphatase” refers to an enzyme that dephosphorylates N-acetylglucosamine-6-phosphate (GlcNAc-6-P) hereby synthesizing N-acetylglucosamine (GlcNAc).

The term “N-acetylmannosamine-6-phosphate phosphatase” refers to an enzyme that dephosphorylates N-acetyl mannosamine-6-phosphate (ManNAc-6P) to N-acetylmannosamine (ManNAc).

The terms “N-acetylneuraminate kinase”, “ManNAc kinase”, “N-acetyl-D-mannosamine kinase” and “nanK” are used interchangeably and refer to an enzyme that phosphorylates ManNAc to synthesize N-acetylmannosamine-phosphate (ManNAc-6-P).

The terms “N-acetylmannosamine-6-phosphate 2-epimerase”, “ManNAc-6-P isomerase”, “ManNAc-6-P 2-epimerase”, N-acetylglucosamine-6P 2-epimerase and “nanE” are used interchangeably and refer to an enzyme that catalyzes the reaction ManNAc-6-P=N-acetylglucosamine-6-phosphate (GlcNAc-6-P).

The terms “phosphoacetylglucosamine mutase”, “acetylglucosamine phosphomutase”, “acetylaminodeoxyglucose phosphomutase”, “phospho-N-acetylglucosamine mutase” and “N-acetyl-D-glucosamine 1,6-phosphomutase” are used interchangeably and refer to an enzyme that catalyses the conversion of N-acetyl-glucosamine 1-phosphate into N-acetylglucosamine 6-phosphate.

The terms “N-acetylglucosamine 1-phosphate uridylyltransferase”, “N-acetylglucosamine-1-phosphate uridyltransferase”, “UDP-N-acetylglucosamine diphosphorylase”, “UDP-N-acetylglucosamine pyrophosphorylase”, “uridine diphosphoacetylglucosamine pyrophosphorylase”, “UTP:2-acetamido-2-deoxy-alpha-D-glucose-1-phosphate uridylyltransferase”, “UDP-GlcNAc pyrophosphorylase”, “GlmU uridylyltransferase”, “Acetylglucosamine 1-phosphate uridylyltransferase”, “UDP-acetylglucosamine pyrophosphorylase”, “uridine diphosphate-N-acetylglucosamine pyrophosphorylase”, “uridine diphosphoacetylglucosamine phosphorylase”, and “acetylglucosamine 1-phosphate uridylyltransferase” are used interchangeably and refer to an enzyme that catalyses the conversion of N-acetylglucosamine 1-phosphate (GlcNAc-1-P) into UDP-N-acetylglucosamine (UDP-GlcNAc) by the transfer of uridine 5-monophosphate (from uridine 5-triphosphate (UTP)).

The term glucosamine-1-phosphate acetyltransferase refers to an enzyme that catalyses the transfer of the acetyl group from acetyl coenzyme A to glucosamine-1-phosphate (GlcN-1-P) to produce N-acetylglucosamine-1-phosphate (GlcNAc-1-P).

The term “glmU” refers to a bifunctional enzyme that has both N-acetylglucosamine-1-phosphate uridyltransferase and glucosamine-1-phosphate acetyltransferase activity and that catalyses two sequential reactions in the de novo biosynthetic pathway for UDP-GlcNAc. The C-terminal domain catalyses the transfer of acetyl group from acetyl coenzyme A to GlcN-1-P to produce GlcNAc-1-P, which is converted into UDP-GlcNAc by the transfer of uridine 5-monophosphate, a reaction catalysed by the N-terminal domain.

The terms “N-acetylneuraminate lyase”, “Neu5Ac lyase”, “N-acetylneuraminate pyruvate-lyase”, “N-acetylneuraminic acid aldolase”, “NALase”, “sialate lyase”, “sialic acid aldolase”, “sialic acid lyase” and “nanA” are used interchangeably and refer to an enzyme that degrades N-acetylneuraminate into N-acetylmannosamine (ManNAc) and pyruvate.

The terms “N-acylneuraminate-9-phosphate synthase”, “N-acylneuraminate-9-phosphate synthetase”, “NANA synthase”, “NANAS”, “NANS”, “NmeNANAS”, “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesise N-acylneuraminate.

The term “glycosyltransferase” as used herein refers to an enzyme capable to catalyse the transfer of sugar moieties from an activated donor molecule to a specific acceptor, forming glycosidic bonds. Said donor can be a precursor as defined herein. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Fucosyltransferases are glycosyltransferases that transfer a fucose residue (Fuc) from a GDP-fucose (GDP-Fuc) donor onto a glycan acceptor. Fucosyltransferases comprise alpha-1,2-fucosyltransferases, alpha-1,3-fucosyltransferases, alpha-1,4-fucosyltransferases, alpha-1,3/4-fucosyltransferases and alpha-1,6-fucosyltransferases that catalyse the transfer of a Fuc residue from GDP-Fuc onto a glycan acceptor via alpha-glycosidic bonds. Fucosyltransferases can be found but are not limited to the GT10, GT11, GT23, GT65 and GT68 CAZy families. Sialyltransferases are glycosyltransferases that transfer a sialic acid (like Neu5Ac or Neu5Gc) from a donor (like CMP-Neu5Ac or CMP-Neu5Gc) onto a glycan acceptor. Sialyltransferases comprise alpha-2,3-sialyltransferases, alpha-2,6-sialyltransferases and alpha-2,8-sialyltransferases that catalyse the transfer of a sialic acid onto a glycan acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from a UDP-galactose (UDP-Gal) donor onto a glycan acceptor. Galactosyltransferases comprise beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases and alpha-1,4-galactosyltransferases that transfer a Gal residue from UDP-Gal onto a glycan acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from a UDP-glucose (UDP-Glc) donor onto a glycan acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto a glycan acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto a glycan acceptor. Mannosyltransferases comprise alpha-1,2-mannosyltransferases, alpha-1,3-mannosyltransferases and alpha-1,6-mannosyltransferases that transfer a Man residue from GDP-Man onto a glycan acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from a UDP-N-acetylglucosamine (UDP-GlcNAc) donor onto a glycan acceptor. N-acetylglucosaminyltransferases can be found but are not limited to GT2 and GT4 CAZy families. Galactoside beta-1,3-N-acetylglucosaminyltransferases are part of N-acetylglucosaminyltransferases and transfer GlcNAc from a UDP-GlcNAc donor onto a terminal galactose unit present in a glycan acceptor via a beta-1,3-linkage. Beta-1,6-N-acetylglucosaminyltransferases are N-acetylglucosaminyltransferases that transfer GlcNAc from a UDP-GlcNAc donor onto a glycan acceptor via a beta-1,6-linkage. N-acetylgalactosaminyltransferases are glycosyltransferases that transfer an N-acetylgalactosamine group (GalNAc) from a UDP-N-acetylgalactosamine (UDP-GalNAc) donor onto a glycan acceptor. N-acetylgalactosaminyltransferases can be found but are not limited to GT7, GT12 and GT27 CAZy families. Alpha-1,3-N-acetylgalactosaminyltransferases are part of the N-acetylgalactosaminyltransferases and transfer GalNAc from a UDP-GalNAc donor to a glycan acceptor via an alpha-1,3-linkage. N-acetylmannosaminyltransferases are glycosyltransferases that transfer an N-acetylmannosamine group (ManNAc) from a UDP-N-acetylmannosamine (UDP-ManNAc) donor onto a glycan acceptor. Xylosyltransferases are glycosyltransferases that transfer a xylose residue (Xyl) from a UDP-xylose (UDP-Xyl) donor onto a glycan acceptor. Xylosyltransferases can be found but are not limited to GT14, GT61 and GT77 CAZy families. Glucuronyltransferases are glycosyltransferases that transfer a glucuronate from a UDP-glucuronate donor onto a glycan acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from a UDP-galacturonate donor onto a glycan acceptor. N-glycolylneuraminyltransferases are glycosyltransferases that transfer an N-glycolylneuraminic acid group (Neu5Gc) from a CMP-Neu5Gc donor onto a glycan acceptor. Rhamnosyltransferases are glycosyltransferases that transfer a rhamnose residue from a GDP-rhamnose donor onto a glycan acceptor. Rhamnosyltransferases can be found but are not limited to the GT1, GT2 and GT102 CAZy families. N-acetylrhamnosyltransferases are glycosyltransferases that transfer an N-acetylrhamnosamine residue from a UDP-N-acetyl-L-rhamnosamine donor onto a glycan acceptor. UDP-4-amino-4,6-dideoxy-N-acetyl-beta-L-altrosamine transaminases are glycosyltransferases that use a UDP-2-acetamido-2,6-dideoxy-L-arabino-4-hexulose in the biosynthesis of pseudaminic acid, which is a sialic acid-like sugar that is used to modify flagellin. UDP-N-acetylglucosamine enolpyruvyl transferases (murA) are glycosyltransferases that transfer an enolpyruvyl group from phosphoenolpyruvate (PEP) to UDP-N-acetylglucosamine (UDPAG) to form UDP-N-acetylglucosamine enolpyruvate. Fucosaminyltransferases are glycosyltransferases that transfer an N-acetylfucosamine residue from a dTDP-N-acetylfucosamine or a UDP-N-acetylfucosamine donor onto a glycan acceptor.

The terms “activated monosaccharide”, “nucleotide-activated sugar”, “nucleotide-sugar”, “activated sugar”, “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), CMP-N-glycolylneuraminic acid (CMP-Neu5Gc), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, GDP-fucose (GDP-Fuc), GDP-rhamnose and UDP-xylose. Nucleotide-sugars act as glycosyl donors in glycosylation reactions. Glycosylation reactions are reactions that are catalysed by glycosyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyra nose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), N-glycolylneuraminic acid, N-acetylgalactosamine (GalNAc), galactosamine (Gain), fucose (Fuc), rhamnose (Rha), glucuronic acid, gluconic acid, fructose (Fru) and polyols. With the term polyol is meant an alcohol containing multiple hydroxyl groups. For example, glycerol, sorbitol, or mannitol.

The term “sugar phosphate” as used herein refers to one of the above listed monosaccharides which is phosphorylated. Examples of sugar phosphates include but are not limited to glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these sugar phosphates are precursors or intermediates for the production of activated monosaccharide.

The term “disaccharide” as used herein refers to a saccharide polymer containing two simple sugars, i.e. monosaccharides. Such disaccharides contain monosaccharides preferably selected from the list of monosaccharides as used herein above. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc).

The term “sialylated disaccharide” as used herein refers to a disaccharide containing two monosaccharides wherein one of said monosaccharides is a sialic acid as defined herein. Examples of sialylated disaccharides comprise Neu5Ac-a2,3-Gal, Neu5Ac-a2,6-Gal and fucopyranosyl-(1-4)-N-glycolylneuraminic acid (Fuc-(1-4)-Neu5Gc).

“Oligosaccharide” as the term is used herein and as generally understood in the state of the art, refers to a saccharide polymer containing a small number, typically three to ten, but used herein three to twenty, of simple sugars, i.e. monosaccharides. Preferably the oligosaccharide as described herein contains monosaccharides selected from the list as used herein above. The oligosaccharide as used in the present invention can be a linear structure or can include branches. The linkage (e.g. glycosidic linkage, galactosidic linkage, glucosidic linkage, etc.) between two sugar units can be expressed, for example, as 1,4, 1≥4, or (1-4), used interchangeably herein. For example, the terms “Gal-b1,4-Glc”, “Gal-β1,4-Glc”, “b-Gal-(1≥4)-Glc”, “β-Gal-(1≥4)-Glc”, “Galbeta1-4-Glc”, “Gal-b(1-4)-Glc” and “Gal-β(1-4)-Glc” have the same meaning, i.e. a beta-glycosidic bond links carbon-1 of galactose (Gal) with the carbon-4 of glucose (Glc). Each monosaccharide can be in the cyclic form (e.g. pyranose or furanose form). Linkages between the individual monosaccharide units may include alpha 1≥2, alpha 1≥3, alpha 1≥4, alpha 1≥6, alpha 2>1, alpha 2≥3, alpha 2≥4, alpha 2≥6, beta 1≥2, beta 1≥3, beta 1≥4, beta 1≥6, beta 2≥1, beta 2≥3, beta 2≥4, and beta 2≥6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars and antigens of the human ABO blood group system. The term “glycan acceptor” as used herein refers to mono-, di- and oligosaccharides as defined herein. As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

A ‘fucosylated oligosaccharide’ as used herein and as generally understood in the state of the art is an oligosaccharide that is carrying a fucose-residue. Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose.

As used herein, a ‘sialylated oligosaccharide’ is to be understood as a negatively charged sialic acid containing oligosaccharide, i.e. an oligosaccharide having a sialic acid residue. It has an acidic nature. Some examples are 3-SL (3′-sialyllactose or 3′SL or Neu5Ac-a2,3-Gal-b1,4-Glc), 3′-sialyllactosamine, 6-SL (6′-sialyllactose or 6′SL or Neu5Ac-a2,6-Gal-b1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′-sialyllactose, SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc β-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc β-1,4(Neu5Acα-2,8Neu5Acα-2,8Neu5Acα2,3)Gal13-1,4Glc, GP1c Neu5Acα-2,8Neu5Acα-2,3Gal13-1,3GalNAc 13-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fuca-1,2Galβ-1,3GalNAcβ-1,4(Neu5Acα-2,3)Gal β-1,4Glc; all of which may be extended to the production of the corresponding gangliosides by reacting the above oligosaccharide moieties with ceramide or synthetizing the above oligosaccharides on a ceramide.

A ‘neutral oligosaccharide’ or a ‘non-charged oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2,3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

Mammalian milk oligosaccharides comprise oligosaccharides present in milk found in any phase during lactation including colostrum milk from humans and mammals including but not limited to cows (Bos Taurus), sheep (Ovis cries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus).

As used herein the term “Lewis-type antigens” comprise the following oligosaccharides: H1 antigen, which is Fucα1-2Galβ1-3GlcNAc, or in short 2′FLNB; Lewisa, which is the trisaccharide Galβ1-3[Fucα1-4]GlcNAc, or in short 4-FLNB; Lewisb, which is the tetrasaccharide Fucα1-2Galβ1-3[Fucα1-4]GlcNAc, or in short DiF-LNB; sialyl Lewisa which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewisx, which is the trisaccharide Galβ1-4[Fucα1-3]GlcNAc, or otherwise known as 3-Fucosyl-N-acetyl-lactosamine, in short 3-FLacNAc, Lewisy, which is the tetrasaccharide Fucα1-2Galβ1-4[Fucα1-3]GlcNAc and sialyl Lewisx which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, the term “O-antigen” refers to the repetitive glycan component of the surface lipopolysaccharide (LPS) of Gram-negative bacteria. The term “lipopolysaccharide” or “LPS” refers to glycolipids found in the outer membrane of Gram-negative bacteria which are composed of a lipid A, a core oligosaccharide and the O-antigen. The term “capsular polysaccharides” refers to long-chain polysaccharides with oligosaccharide repeat structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components consist of alternating residues of beta-1,4 linked GlcNAc and N-acetylmuramic acid. The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. Said structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

The terms “LNT II”, “LNT-II”, “LN3”, “lacto-N-triose II”, “lacto-N-triose II”, “lacto-N-triose”, “lacto-N-triose” or “GlcNAcβ1-3Galβ1-4Glc” as used in the present invention, are used interchangeably.

The terms “LNT”, “lacto-N-tetraose”, “lacto-N-tetraose” or “Gal31-3GlcNAcβ1-3Galβ1-4Glc” as used in the present invention, are used interchangeably.

The terms “LNnT”, “lacto-N-neotetraose”, “lacto-N-neotetraose”, “neo-LNT” or “Galβ1-4GlcNAcβ1-3Galβ1-4Glc” as used in the present invention, are used interchangeably.

The terms “LSTa”, “LS-Tetrasaccharide a”, “Sialyl-lacto-N-tetraose a”, “sialyllacto-N-tetraose a” or “Neu5Ac-a2,3-Gal-b1,3-GlcNAc-b1,3-Gal-b1,4-Glc” as used in the present invention, are used interchangeably.

The terms “LSTb”, “LS-Tetrasaccharide b”, “Sialyl-lacto-N-tetraose b”, “sialyllacto-N-tetraose b” or “Gal-b1,3-(Neu5Ac-a2,6)-GlcNAc-b1,3-Gal-b1,4-Glc” as used in the present invention, are used interchangeably.

The terms “LSTc”, “LS-Tetrasaccharide c”, “Sialyl-lacto-N-tetraose c”, “sialyllacto-N-tetraose c”, “sialyllacto-N-neotetraose c” or “Neu5Ac-a2,6-Gal-b1,4-GlcNAc-b1,3-Gal-b1,4-Glc” as used in the present invention, are used interchangeably.

The terms “LSTd”, “LS-Tetrasaccharide d”, “Sialyl-lacto-N-tetraose d”, “sialyllacto-N-tetraose d”, “sialyllacto-N-neotetraose d” or “Neu5Ac-a2,3-Gal-b1,4-GlcNAc-b1,3-Gal-b1,4-Glc” as used in the present invention, are used interchangeably.

The terms “cell genetically modified for the production of a sialylated di- and/or oligosaccharide” or “cell metabolically engineered for the production of a sialylated di- and/or oligosaccharide” within the context of the present disclosure refers to a cell of a microorganism which is genetically manipulated to comprise at least one sialyltransferase combined with any one or more of i) a gene encoding a glycosyltransferase necessary for the synthesis of said sialylated di- and/or oligosaccharide, ii) a biosynthetic pathway to produce a nucleotide donor suitable to be transferred by said glycosyltransferase to a carbohydrate precursor, and/or iii) a biosynthetic pathway to produce a precursor or a mechanism of internalization of a precursor from the culture medium into the cell where it is glycosylated to produce the sialylated di- and/or oligosaccharide.

The term “pathway for production of a sialylated di- and/or oligosaccharide” as used herein is a biochemical pathway consisting of the enzymes and their respective genes involved in the synthesis of a sialylated di- and/or oligosaccharide as defined herein. Said pathway for production of a sialylated di- and/or oligosaccharide comprises at least one sialyltransferase. Furthermore, said pathway for production of a sialylated di- and/or oligosaccharide can comprise but is not limited to pathways involved in the synthesis of a nucleotide-activated sugar and the transfer of said nucleotide-activated sugar to an acceptor to create a sialylated di- and/or oligosaccharide of the present invention. Examples of such pathway comprise but are not limited to a fucosylation, sialylation, galactosylation, N-acetylglucosaminylation, N-acetylgalactosylation, mannosylation, N-acetylmannosaminylation pathway. A ‘fucosylation pathway’ as used herein is a biochemical pathway comprising at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-fucokinase/GDP-fucose pyrophosphorylase combined with a fucosyltransferase leading to α 1,2; α 1,3; α 1,4 and/or α 1,6 fucosylated oligosaccharides.

A ‘sialylation pathway’ is a biochemical pathway comprising at least one of the enzymes and their respective genes chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthetase, phosphatase, N-acetylneuraminate synthase and N-acylneuraminate cytidylyltransferase combined with a sialyltransferase leading to α 2,3; α 2,6 and/or α 2,8 sialylated oligosaccharides.

A ‘galactosylation pathway’ as used herein is a biochemical pathway comprising at least one of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase combined with a galactosyltransferase leading to a galactosylated compound comprising a mono-, di-, or oligosaccharide having an alpha or beta bound galactose on any one or more of the 2, 3, 4 and 6 hydroxyl group of said mono-, di-, or oligosaccharide.

An ‘N-acetylglucosaminylation pathway’ as used herein is a biochemical pathway comprising at least one of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase combined with a glycosyltransferase leading to a GlcNAc-modified compound comprising a mono-, di-, or oligosaccharide having an alpha or beta bound N-acetylglucosamine (GlcNAc) on any one or more of the 3, 4 and 6 hydroxyl group of said mono-, di- or oligosaccharide.

An ‘N-acetylgalactosaminylation pathway’ as used herein is a biochemical pathway comprising at least one of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and UDP-GalNAc pyrophosphorylase combined with a glycosyltransferase leading to a GalNAc-modified compound comprising a mono-, di- or oligosaccharide having an alpha or beta bound N-acetylgalactosamine on said mono-, di- or oligosaccharide.

A ‘mannosylation pathway’ as used herein is a biochemical pathway comprising at least one of the enzymes and their respective genes chosen from the list comprising mannose-6-phosphate isomerase, phosphomannomutase and mannose-1-phosphate guanylyltransferase combined with a glycosyltransferase leading to a mannosylated compound comprising a mono-, di- or oligosaccharide having an alpha or beta bound mannose on said mono-, di- or oligosaccharide.

An ‘N-acetylmannosaminylation pathway’ as used herein is a biochemical pathway comprising at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridylyltransferase, glucosamine-1-phosphate acetyltransferase, glucosamine-1-phosphate acetyltransferase, UDP-GlcNAc 2-epimerase and ManNAc kinase combined with a glycosyltransferase leading to a ManNAc-modified compound comprising a mono-, di- or oligosaccharide having an alpha or beta bound N-acetylmannosamine on said mono-, di- or oligosaccharide.

The term “sialic acid transporter” as used herein refers to a protein that is capable to transport a sialic acid as defined herein across the cytoplasm and/or the cell wall, i.e. in and/or out of the cell. Said transport can be import into and/or export out of the cell.

Such sialic acid transporters used in the present invention can be porters or P—P-bond-hydrolysis-driven transporters as defined by the Transporter Classification Database that is operated and curated by the Saier Lab Bioinformatics Group available via www.tcdb.org. This Transporter Classification Database details a comprehensive IUBMB approved functional and phylogenetic classification system for transport proteins known as the Transporter Classification (TC) system. The TCDB classification searches as described here are defined based on TCDB.org as released on 17 Jun. 2019.

Porters is the collective name of uniporters, symporters, and antiporters that utilize a carrier-mediated process (Saier et al., Nucleic Acids Res. 44 (2016) D372-D379). They belong to the electrochemical potential-driven transporters and are also known as secondary carrier-type facilitators. Transport proteins are included in this class when they utilize a carrier-mediated process to catalyse uniport when a single species is transported either by facilitated diffusion or in a membrane potential-dependent process if the solute is charged; antiport when two or more species are transported in opposite directions in a tightly coupled process, not coupled to a direct form of energy other than chemiosmotic energy; and/or symport when two or more species are transported together in the same direction in a tightly coupled process, not coupled to a direct form of energy other than chemiosmotic energy of secondary carriers (Forrest et al., Biochim. Biophys. Acta 1807 (2011) 167-188). These systems are usually stereospecific. Solute:solute countertransport is a characteristic feature of secondary carriers. The dynamic association of porters and enzymes creates functional membrane transport metabolons that channel substrates typically obtained from the extracellular compartment directly into their cellular metabolism (Moraes and Reithmeier, Biochim. Biophys. Acta 1818 (2012), 2687-2706). Solutes that are transported via this porter system include but are not limited to cations, organic anions, inorganic anions, nucleosides, amino acids, polyols, phosphorylated glycolytic intermediates, osmolytes, siderophores.

Transport proteins are included in the class of P—P-bond hydrolysis-driven transporters if they hydrolyse the diphosphate bond of inorganic pyrophosphate, ATP, or another nucleoside triphosphate, to drive the active uptake and/or extrusion of a solute or solutes (Saier et al., Nucleic Acids Res. 44 (2016) D372-D379).

The transporter protein may or may not be transiently phosphorylated, but the substrate is not phosphorylated. Substrates that are transported via the class of P—P-bond hydrolysis-driven transporters include but are not limited to cations, heavy metals, beta-glucan, UDP-glucose, lipopolysaccharides, teichoic acid, sialic acid.

The term “enabled efflux” means to introduce the activity of transport of a solute over the cytoplasm membrane and/or the cell wall. Said transport may be enabled by introducing and/or increasing the expression of a transporter protein as described in the present invention. The term “enhanced efflux” means to improve the activity of transport of a solute over the cytoplasm membrane and/or the cell wall. Said transport may be enhanced by introducing and/or increasing the expression of a transporter protein as described in the present invention. “Expression” of a transporter protein is defined as “overexpression” of the gene encoding said transporter protein in the case said gene is an endogenous gene or “expression” in the case the gene encoding said transporter protein is a heterologous gene that is not present in the wild type strain.

The terms “acetyl-coenzyme A synthetase”, “acs”, “AcCoA synthetase”, “acetat-CoA ligase”, “acyl-activating enzyme” and “yfaC” are used interchangeably and refer to an enzyme that catalyses the conversion of acetate into acetyl-coezyme A (AcCoA) in an ATP-dependent reaction.

The terms “pyruvate dehydrogenase”, “pyruvate oxidase”, “PDX”, “poxB” and “pyruvate:ubiquinone-8 oxidoreductase” are used interchangeably and refer to an enzyme that catalyses the oxidative decarboxylation of pyruvate to produce acetate and CO2.

The terms “lactate dehydrogenase”, “D-lactate dehydrogenase”, “IdhA”, “hslI”, “htpH”, “D-LDH”, “fermentative lactate dehydrogenase” and “D-specific 2-hydroxyacid dehydrogenase” are used interchangeably and refer to an enzyme that catalyses the conversion of lactate into pyruvate hereby generating NADH.

As used herein, the term “cell productivity index (CPI)” refers to the mass of the product produced by the cells divided by the mass of the cells produced in the culture or cultivation.

The term “purified” refers to material that is substantially or essentially free from components which interfere with the activity of the biological molecule. For cells, saccharides, nucleic acids, and polypeptides, the term “purified” refers to material that is substantially or essentially free from components which normally accompany the material as found in its native state. Typically, purified saccharides, oligosaccharides, proteins or nucleic acids of the invention are at least about 50%, 55%, 60%, 65%, 70%, 75%, 80% or 85% pure, usually at least about 90%, 91% 92% 93% 94% 95% 96%, 97%, 98%, or 99% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy.

The terms “cultivation” and “fermentation” are used interchangeably and refer to the culture medium wherein the cell is cultivated or fermented, the cell itself, and the sialylated di- and/or oligosaccharide that is produced by the cell in whole broth, i.e. inside (intracellularly) as well as outside (extracellularly) of the cell.

The term “reactor” refers to the recipient filled with the cultivation. Examples of reactors comprise but are not limited to microfluidic devices, well plates, tubes, shake flasks, fermenters, bioreactors, process vessels, cell culture incubators, CO2 incubators.

The term “precursor” as used herein refers to substances which are taken up or synthetized by the cell for the specific production of sialylated di- and/or oligosaccharide according to the present invention. In this sense a precursor can be an acceptor as defined herein, but can also be another substance, metabolite, which is first modified within the cell as part of the biochemical synthesis route of the sialylated di- and/or oligosaccharide. The term “precursor” as used herein is also to be understood as a chemical compound that participates in a chemical or enzymatic reaction to produce another compound like e.g. an intermediate or an acceptor as defined herein, as part in the metabolic pathway of a sialylated di- and/or oligosaccharide. The term “precursor” as used herein is also to be understood as a donor that is used by a glycosyltransferase to modify an acceptor as defined herein with a sugar moiety in a glycosidic bond, as part in the metabolic pathway of a sialylated di- and/or oligosaccharide. Examples of such precursors comprise the acceptors as defined herein, and/or dihydroxyacetone, glucosamine, N-acetylglucosamine, N-acetylmannosamine, galactosamine, N-acetylgalactosamine, galactosyllactose, phosphorylated sugars like e.g. but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetylglucosamine-6-phosphate, N-acetylmannosamine-6-phosphate, N-acetylglucosamine-1-phosphate, N-acetylneuraminic acid-9-phosphate and nucleotide-activated sugars as defined herein like e.g. UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell is transformed to comprise and to express at least one nucleic acid sequence encoding a protein selected from the group consisting of lactose transporter, fucose transporter, transporter for a nucleotide-activated sugar wherein said transporter internalizes a to the medium added precursor for the synthesis of the sialylated di- and/or oligosaccharide of present invention.

The term “acceptor” as used herein refers to a mono-, di- or oligosaccharide which can be modified by a glycosyltransferase. Examples of such acceptors comprise glucose, galactose, fructose, glycerol, sialic acid, fucose, mannose, maltose, sucrose, lactose, lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof.

### DETAILED DESCRIPTION OF THE INVENTION

According to a first embodiment, the present invention provides a metabolically engineered cell of a microorganism for the production of a sialylated di- and/or oligosaccharide. Herein, a metabolically engineered cell of a microorganism comprising a pathway for the production of a sialylated di- and/or oligosaccharide is provided which synthesizes sialic acid, expresses at least one sialyltransferase, is modified to have a fully or partially knocked out or rendered less functional sialic acid catabolic pathway and is modified for overexpression of an endogenous sialic acid transporter and/or expression of an exogenous, homologous and/or heterologous sialic acid transporter. Said cell is preferably modified in the expression or activity of any one of said sialyltransferase.

According to a second embodiment, the present invention provides a method for the production of a sialylated di- and/or oligosaccharide by a metabolically engineered cell of a microorganism. The method comprises the steps of:
1) providing a cell of a microorganism as described herein, and
2) cultivating said cell under conditions permissive to produce said sialylated di- and/or oligosaccharide.

Preferably, the sialylated di- and/or oligosaccharide is separated from the cultivation as explained herein.

In the scope of the present invention, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 2.0-10.0, preferably 7+/−3.

The present invention provides different types of cells for the production of a sialylated di- and/or oligosaccharide with a metabolically engineered cell of a microorganism.

In a preferred aspect of the method and/or cell of the invention, the metabolically engineered cell is modified with gene expression modules wherein the expression from any one of said expression modules is constitutive or is tuneable.

Said expression modules are also known as transcriptional units and comprise polynucleotides for expression of recombinant genes including coding gene sequences and appropriate transcriptional and/or translational control signals that are operably linked to the coding genes. Said control signals comprise promoter sequences, untranslated regions, ribosome binding sites, terminator sequences. Said expression modules can contain elements for expression of one single recombinant gene but can also contain elements for expression of more recombinant genes or can be organized in an operon structure for integrated expression of two or more recombinant genes. Said polynucleotides may be produced by recombinant DNA technology using techniques well-known in the art. Methods which are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred aspect of the present invention, the cell is modified with one or more expression modules. The expression modules can be integrated in the genome of said cell or can be presented to said cell on a vector. Said vector can be present in the form of a plasmid, cosmid, phage, liposome, or virus, which is to be stably transformed/transfected into said metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the invention. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. Said recombinant gene is involved in the expression of a polypeptide acting in the synthesis of said sialylated di- and/or oligosaccharide; or said recombinant gene is linked to other pathways in said host cell that are not involved in the synthesis of said sialylated di- and/or oligosaccharide. Said recombinant genes encode endogenous proteins with a modified expression or activity, preferably said endogenous proteins are overexpressed; or said recombinant genes encode heterologous proteins that are heterogeneously introduced and expressed in said modified cell, preferably overexpressed. The endogenous proteins can have a modified expression in the cell which also expresses a heterologous protein.

According to one aspect of the method and/or cell of the invention, the cell is metabolically engineered to comprise a pathway for the production of a sialylated di- and/or oligosaccharide. In a preferred embodiment of the method and/or cell of the invention, the pathway for the production of a sialylated di- and/or oligosaccharide comprises a sialylation pathway. In a more preferred embodiment of the method and/or cell of the invention, the sialylation pathway comprises at least one sialyltransferase. In a preferred embodiment of the method and/or cell, the sialyltransferase is chosen from the list comprising alpha-2,3-sialyltransferase, alpha-2,6-sialyltransferase, and alpha-2,8-sialyltransferase. In another and/or additional preferred embodiment of the method and/or cell, the cell expresses more than one sialyltransferase that synthesize any one or more sialylated di- and/or oligosaccharides as defined herein. According to another more preferred embodiment of the method and/or cell of the invention, the sialylation pathway further comprises an N-acylneuraminate cytidylyltransferase.

According to the method and/or cell of the invention, the cell synthesizes sialic acid as defined herein. In a preferred embodiment of the method and/or cell of the invention, the cell comprises a pathway for production of sialic acid. In a more preferred embodiment of the method and/or cell of the invention, the pathway for production of sialic acid comprises any one or more of the enzymes chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthetase, phosphatase, N-acetylneuraminate synthase and N-acylneuraminate cytidylyltransferase.

In another and/or additional preferred embodiment of the method and/or cell of the invention, the cell uses the synthesized sialic acid for the production of a sialylated di- and/or oligosaccharide.

In a preferred embodiment, the cell comprises a sialylation pathway for production of a sialylated di- and/or oligosaccharide wherein said cell expresses at least one enzyme chosen from the list comprising an N-acylglucosamine 2-epimerase like is known e.g. from several species including Bacteroides ovatus, E. coli, Homo sapiens, Rattus norvegicus, an N-acetylneuraminate synthase like is known e.g. from Neisseria meningitidis or Campylobacter jejuni, an N-acylneuraminate cytidylyltransferase like is known e.g from N. meningitidis, and a sialyltransferase including an alpha-2,3-sialyltransferase, an alpha-2,6-sialyltransferase and/or an alpha-2,8-sialyltransferase, wherein the enzymes are as defined herein. N-acyl-D-glucosamine (GlcNAc) can be added to the cell and/or can be provided by an enzyme expressed in the cell or by the mechanism of the cell. Such cell producing GlcNAc can express a phosphatase converting GlcNAc-6-phosphate into GlcNAc, like any one or more of e.g. the E. coli HAD-like phosphatase genes comprising aphA, Cof, HisB, OtsB, SurE, Yaed, YcjU, YedP, YfbT, YidA, YigB, YihX, YniC, YgaB, YrbL, AppA, Gph, SerB, YbhA, YbiV, YbjL, Yfb, YieH, YjgL, YjjG, YrfG and YbiU, PsMupP from Pseudomonas putida, ScDOG1 from S. cerevisiae and BsAraL from Bacillus subtilis as described in WO18122225. Preferably, the cell is modified to produce GlcNAc. More preferably, the cell is modified for enhanced GlcNAc production. Said modification can be any one or more chosen from the group comprising knockout of a glucosamine-6-phosphate deaminase, an N-acetylglucosamine-6-phosphate deacetylase and/or an N-acetyl-D-glucosamine kinase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a sialylated di- and/or oligosaccharide wherein said cell expresses at least one enzyme chosen from the list comprising a UDP-N-acetylglucosamine 2-epimerase like is known e.g. from several species including C. jejuni, E. coli, N. meningitidis, Bacillus subtilis, Citrobacter rodentium, an N-acetylneuraminate synthase like is known e.g. from N. meningitidis or C. jejuni, an N-acylneuraminate cytidylyltransferase like is known e.g. from N. meningitidis, and a sialyltransferase including an alpha-2,3-sialyltransferase, an alpha-2,6-sialyltransferase and/or an alpha-2,8-sialyltransferase, wherein the enzymes are as defined herein. UDP-N-acetylglucosamine (UDP-GlcNAc) can be added to the cell and/or can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be any one or more enzymes chosen from the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

In an alternative and/or additional preferred embodiment, the cell comprises a pathway for production of a sialylated di- and/or oligosaccharide wherein said cell expresses at least one enzyme chosen from the list comprising an N-acetylmannosamine-6-phosphate 2-epimerase like is known e.g. from several species including E. coli, Haemophilus influenzae, Enterobacter sp., Streptomyces sp., an N-acylneuraminate-9-phosphate synthetase, an N-acylneuraminate-9-phosphatase like is known e.g. from Candidatus Magnetomorum sp. HK-1 or Bacteroides thetaiotaomicron, an N-acetylneuraminate synthase like is known e.g. from N. meningitidis or C. jejuni, an N-acylneuraminate cytidylyltransferase like is known e.g from N. meningitidis, and a sialyltransferase including an alpha-2,3-sialyltransferase, an alpha-2,6-sialyltransferase and/or an alpha-2,8-sialyltransferase, wherein the enzymes are as defined herein. N-acetyl-D-glucosamine 6-phosphate (GlcNAc-6P) can be added to the cell and/or can be provided by an enzyme expressed in the cell or by the metabolism of the cell. Such cell producing GlcNAc-6P can express an enzyme converting, e.g., GlcN6P, which is to be added to the cell, to GlcNAc-6P. This enzyme may be a glucosamine 6-phosphate N-acetyltransferase from several species including Saccharomyces cerevisiae, Kluyveromyces lactis, Homo sapiens. Preferably, the cell is modified to produce GlcNAc-6P. More preferably, the cell is modified for enhanced GlcNAc-6P production. Said modification can be any one or more chosen from the group comprising knockout of a glucosamine-6-phosphate deaminase, an N-acetylglucosamine-6-phosphate deacetylase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a sialylated di- and/or oligosaccharide wherein said cell expresses at least one enzyme chosen from the list comprising a bifunctional UDP-GlcNAc 2-epimerase/kinase like is known e.g. from several species including H. sapiens, R. norvegicus and Mus musculus, an N-acylneuraminate-9-phosphate synthetase, an N-acylneuraminate-9-phosphatase like is known e.g. from Candidatus Magnetomorum sp. HK-1 or Bacteroides thetaiotaomicron, an N-acetylneuraminate synthase like is known e.g. from N. meningitidis or C. jejuni, an N-acylneuraminate cytidylyltransferase like is known e.g from N. meningitidis, and a sialyltransferase including an alpha-2,3-sialyltransferase, an alpha-2,6-sialyltransferase and/or an alpha-2,8-sialyltransferase, wherein the enzymes are as defined herein. UDP-N-acetylglucosamine can be added to the cell and/or can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including H. sapiens, E. coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the host cell used herein is optionally genetically engineered to import an acceptor in the cell, by the introduction and/or overexpression of a transporter able to import the respective acceptor in the cell. Such transporter is for example a membrane protein belonging to the major facilitator superfamily (MFS), the ATP-binding cassette (ABC) transporter family or the PTS system involved in the uptake of e.g. mono-, di- and/or oligosaccharides.

Additionally, or alternatively, the host cell used herein is optionally genetically engineered to import lactose in the cell, by the introduction and/or overexpression of a lactose permease. Said lactose permease is for example encoded by the lacy gene or the lac12 gene.

According to the method and/or cell of the invention, the cell is modified to have a fully or partially knocked out or rendered less functional sialic acid catabolic pathway. In a preferred embodiment of the method and/or cell, the cell is modified to have any one or more fully or partially knocked out genes comprising N-acetylneuraminate lyase, N-acetylneuraminate epimerase, N-acetylneuraminate kinase, N-acetylglucosamine-6-P deacetylase and glucosamine-6-P deaminase. In another preferred embodiment of the method and/or cell, the cell is modified to have one or more mutation(s) in any one of the proteins comprising N-acetylneuraminate lyase, N-acetylneuraminate epimerase, N-acetylneuraminate kinase, N-acetylglucosamine-6-P deacetylase and glucosamine-6-P deaminase wherein said one or more mutation(s) render said proteins less functional or dysfunctional.

According to the method and/or cell of the invention, the cell expresses a sialic acid transporter. The sialic acid transporter is either an endogenous protein with a modified expression, preferably said endogenous protein is overexpressed; or the sialic acid transporter is an exogenous, homologous and/or heterologous protein, which can be heterologously expressed by the cell. The heterologously expressed sialic acid transporter will then be introduced and expressed, preferably overexpressed. In another embodiment, the endogenous sialic acid transporter can have a modified expression in the cell which also expresses an exogenous, homologous and/or heterologous sialic acid transporter. In another embodiment, modified expression of an endogenous sialic acid transporter comprises modified expression of other proteins that map in the same operon of said endogenous sialic acid transporter and/or share common control sequences for expression. In another embodiment, the sialic acid transporter is expressed together with conterminal proteins that share the same regulon. In another embodiment, when the sialic acid transporter is an inner membrane transporter (complex), the sialic acid transporter is expressed together with one or more outer membrane transporter(s). In an alternative embodiment, when the sialic acid transporter is an outer membrane transporter, the sialic acid transporter is expressed together with one or more inner membrane protein(s). In an alternative embodiment, the sialic acid transporter is expressed with one or more inner sialic acid transporter and/or one or more outer sialic acid transporter. According to a further aspect of the invention, the polynucleotide encoding the sialic acid transporter is adapted to the codon usage of the respective cell or expression system.

In a preferred aspect of the method and/or cell of the invention, the sialic acid transporter is selected from the group of porters and P—P-bond-hydrolysis-driven transporters.

In a preferred aspect of the method and/or cell, when the sialic acid transporter is selected from the group of porters, the sialic acid transporter is selected from the group of TCDB classes 2.A.1.12, 2.A.21.3 and 2.A.56.1.

In a preferred aspect of the method and/or cell, when the sialic acid transporter is selected from the group of porters, the sialic acid transporter is selected from the group of eggnog families 1MU0F, 1MUNB, 1MVHH, 1MWKH, 1R4VK, 1RJ8K, 1RMH7, 1RNAM, 1RPTE, 1RQFS, 1RZ3Z, 1SA02, 1TPNU, 1TPVE, 1TQCK, 1XTIW, 1XU43, 1Y733, 1Y7P0, 1YAT3, 258Y6, 25E9U, 25SBT, 25Y42, 26G88, 27G1B, 2IX6M, 2TQBC, 2TQW5, 36UQA, 3788V, 3F50Z, 3WG4Q, 3WP13, 3WTAI, 3WVF7, 3X1B1, 3XP7A, 3ZJ4T, 404JV, 41EHH, 4BDTU, 4C594, 4GX8Z, 4GY09, 4H21M, 4H9KW, 4H9WK, 4HB5K, 4IEK0, COG0477, COG0591, COG1593, COG2704, COG2814, COG3055, COG3090 and COG4666.

In another preferred aspect of the method and/or cell, when the sialic acid transporter is selected from the group of porters, the sialic acid transporter is selected from the PFAM list PF00083, PF00474, PF04290, PF06808, PF07690 and PF11874.

In another preferred aspect of the method and/or cell, when the sialic acid transporter is selected from the group of porters, the sialic acid transporter is selected from the interpro list IPR001734, IPR004681, IPR004742, IPR005828, IPR005829, IPR007387, IPR010656, IPR011701, IPR011851, IPR011853, IPR015915, IPR018212, IPR020846, IPR021814, IPR025966, IPR036259 and IPR038377.

In another preferred aspect of the method and/or cell, when the sialic acid transporter is selected from the group of porters, the sialic acid transporter is selected from NanT from Escherichia coli with SEQ ID NO 01, nanT from Yersinia enterocolitica subsp. palearctica serotype O:3 with SEQ ID NO 02, nanT from Buttiauxella brennerae ATCC 51605 with SEQ ID NO 03, nanT from E. coli KTE75 with SEQ ID NO 04, or a transporter from Mycoplasma alligatoris A21JP2 with SEQ ID NO 05, from M. capricolum subsp. capricolum with SEQ ID NO 06, from Staphylococcus aureus with SEQ ID NO 07, from Gemella sp. with SEQ ID NO 08, from Clostridioides difficile ATCC 9689 with SEQ ID NO 09, from Peptostreptococcus russellii with SEQ ID NO 10, from Bacillus obstructivus with SEQ ID NO 11, from S. aureus (strain MW2) with SEQ ID NO 12, from Staphylococcus sp. HMSC070A03 with SEQ ID NO 13, from Cetobacterium ceti with SEQ ID NO 14, from S. argensis with SEQ ID NO 15, from S. schleiferi with SEQ ID NO 16, from Alloiococcus otitis ATCC 51267 with SEQ ID NO 17, from Gracilibacillus dipsosauri with SEQ ID NO 18, from Lactobacillus versmoldensis with SEQ ID NO 19, from Lactobacillus sp. LL6 with SEQ ID NO 20, from Agrilactobacillus composti with SEQ ID NO 21, from L. crispatus with SEQ ID NO 22, from S. pasteuri with SEQ ID NO 23, from L. salivarius cp400 with SEQ ID NO 24, from L. apodemi with SEQ ID NO 25, from Selenomonas sp. oral taxon 478 with SEQ ID NO 26, from Paeniclostridium sordellii with SEQ ID NO 27, from Butyribacterium methylotrophicum with SEQ ID NO 28, from Alkalihalobacillus pseudofirmus with SEQ ID NO 29, from Streptococcus mitis (strain B6) with SEQ ID NO 30, from Aerococcus viridans with SEQ ID NO 31, from Anaerobiospirillum thomasii with SEQ ID NO 32, from Clostridium haemolyticum NCTC 8350 with SEQ ID NO 33, from C. thermobutyricum with SEQ ID NO 34 or SEQ ID NO 35, from Romboutsia ilealis with SEQ ID NO 36, from Megamonas funiformis YIT 11815 with SEQ ID NO 37, from Lysinibacillus sphaericus with SEQ ID NO 38, from uncultured Clostridium sp. with SEQ ID NO 39, from C. hiranonis with SEQ ID NO 40, from C. niameyense with SEQ ID NO 41, from Candidatus Arthromitus sp. SFB-turkey with SEQ ID NO 42, siaT_5 from Suttonella ornithocola with SEQ ID NO 43, a transporter from Pasteurella multocida subsp. multocida OH4807 with SEQ ID NO 44, from P. skyensis with SEQ ID NO 45, from [Haemophilus] felis with SEQ ID NO 46, from Gammaproteobacteria bacterium with SEQ ID NO 47, from [Pasteurella] mairii with SEQ ID NO 48, from Suttonella indologenes with SEQ ID NO 49, from Fusobacterium canifelinum with SEQ ID NO 50, from F. varium with SEQ ID NO 51, from F. necrophorum with SEQ ID NO 52, from Cardiobacterium hominis with SEQ ID NO 53, from Aggregatibacter actinomycetemcomitans RhAA1 with SEQ ID NO 54, or from P. bettyae CCUG 2042 with SEQ ID NO 55, or functional homolog, variant or derivative of any one of the SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 having at least 80% overall sequence identity to the full-length sequence of any one of said polypeptides with SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 respectively and having sialic acid transporter activity.

In a further preferred aspect of the method and/or cell, when the sialic acid transporter is selected from the group of porters, the sialic acid transporter is selected from the porters NanT from Escherichia coli with SEQ ID NO 01, nanT from Yersinia enterocolitica subsp. palearctica serotype O:3 with SEQ ID NO 02, nanT from Buttiauxella brennerae ATCC 51605 with SEQ ID NO 03, nanT from E. coli KTE75 with SEQ ID NO 04, from L. salivarius cp400 with SEQ ID NO 24, or siaT_5 from Suttonella ornithocola with SEQ ID NO 43, or functional homolog, variant or derivative of any one of the SEQ ID NOs 01, 02, 03, 04, 24, or 43 having at least 80% overall sequence identity to the full-length sequence of any one of said preferred polypeptides with SEQ ID NOs 01, 02, 03, 04, 24, or 43, respectively and having sialic acid transporter activity.

In a preferred aspect of the method and/or cell, when the sialic acid transporter is selected from the group of P—P-bond-hydrolysis-driven transporters, the sialic acid transporter is selected from the TCDB class 3.A. 1.5.

In a preferred aspect of the method and/or cell, when the sialic acid transporter is selected from the group of P—P-bond-hydrolysis-driven transporters, the sialic acid transporter is selected from the group of eggnog families 1MU8Z, 1MUZH, 1NU4K, 1R4 KB, 1RNFB, 1RS1R, 1SKPD, 1SMBI, 1Y72G, 1Y72H, 1Y7EZ, 1Y7FQ, COG0444, COG0601, COG0747, COG1173 and COG4608.

In another preferred aspect of the method and/or cell, when the sialic acid transporter is selected from the group of P—P-bond-hydrolysis-driven transporters, the sialic acid transporter is selected from the PFAM list PF00005, PF00496, PF00528 and PF08352.

In another preferred aspect of the method and/or cell, when the sialic acid transporter is selected from the group of P—P-bond-hydrolysis-driven transporters, the sialic acid transporter is selected from the interpro list IPR000515, IPR000914, IPR003439, IPR003593, IPR006311, IPR013563, IPR017871, IPR027417, IPR030678, IPR035906 and IPR039424.

In another preferred aspect of the method and/or cell, when the sialic acid transporter is selected from the group of P—P-bond-hydrolysis-driven transporters, the sialic acid transporter is selected from the ABC transporter from Haemophilus paracuniculus with SEQ ID NO 56, 57, 58 or 59, or functional homolog, variant or derivative of any one of the SEQ ID NOs 56, 57, 58 or 59 having at least 80% overall sequence identity to the full-length sequence of any one of the polypeptides with SEQ ID NOs 56, 57, 58 or 59, respectively and having sialic acid transporter activity.

Said TCDB classes are classified as defined on TCDB.org as released on 17 Jun. 2019. Said eggnog families are classified as defined on eggnogdb 5.0.0 as released on November 2018. Said PFAM list is classified as defined on Pfam 32.0 as released on September 2018. Said interpro lists are as defined by InterPro 75.0 as released on 4 Jul. 2019.

As used herein, a protein having an amino acid sequence having at least 80% sequence identity to the full-length sequence of any of the enlisted sialic acid transporters, is to be understood as that the sequence has 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90% sequence identity to the full-length of the amino acid sequence of the respective sialic acid transporter and having sialic acid transporter activity. The amino acid sequence of such sialic acid transporter can be a sequence chosen from SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59 of the attached sequence listing, or an amino acid sequence that has least 80% sequence identity, 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90% sequence identity to the full-length amino acid sequence of any one of SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, respectively, and having sialic acid transporter activity.

According to another preferred aspect of the method and/or cell of the invention, the sialic acid transporter is a transporter protein involved in the influx and/or efflux of sialic acid across the cytoplasmic membrane, i.e. in and/or out of the cell.

According to another preferred aspect of the method and/or cell of the invention, the cell expresses more than one sialic acid transporter.

In a more preferred alternative embodiment, when said sialic acid transporter is the ABC transporter from Haemophilus paracuniculus with SEQ ID NO 56, said ABC transporter is expressed together with the ABC transporters from H. paracuniculus with SEQ ID NOs 57, 58 and 59.

In a more preferred alternative embodiment, when said sialic acid transporter is the ABC transporter from Haemophilus paracuniculus with SEQ ID NO 57, said ABC transporter is expressed together with the ABC transporters from H. paracuniculus with SEQ ID NOs 56, 58 and 59.

In a more preferred alternative embodiment, when said sialic acid transporter is the ABC transporter from Haemophilus paracuniculus with SEQ ID NO 58, said ABC transporter is expressed together with the ABC transporters from H. paracuniculus with SEQ ID NOs 56, 57 and 59.

In a more preferred alternative embodiment, when said sialic acid transporter is the ABC transporter from Haemophilus paracuniculus with SEQ ID NO 59, said ABC transporter is expressed together with the ABC transporters from H. paracuniculus with SEQ ID NOs 56, 57 and 58.

In a further aspect of the method and/or cell of the present invention, the host cell expresses a membrane protein that is a transporter protein involved in transport of compounds across the outer membrane of the cell wall, it means in and/or out of the cell. Preferably the cell is transformed to comprise at least one nucleic acid sequence encoding a protein selected from the group comprising a lactose transporter, a glucose transporter, a galactose transporter or a transporter for a nucleotide-activated sugar like for example a transporter for UDP-GlcNAc.

In another and/or additional further aspect of the method and/or cell of the present invention, the host cell expresses a membrane protein that is a transporter protein involved in transport of the sialylated di- and/or oligosaccharide across the outer membrane of the cell wall, it means out of the cell. Preferably the cell is transformed to comprise at least one nucleic acid sequence encoding a membrane transporter protein selected from the group comprising a siderophore exporter, a major facilitator superfamily (MFS) transporter, an ATP-binding cassette (ABC) transporter or a sugar efflux transporter.

According to a preferred aspect of the method and/or cell of the invention, >95% of the sialic acid that is synthesized by the cell is used for the production of a sialylated di- and/or oligosaccharide.

According to another preferred aspect of the method and/or cell of the present invention, the cell is capable to synthesize N-acetylmannosamine (ManNAc), N-acetyl mannosamine-6-phosphate (ManNAc-6-phosphate) and/or phosphoenolpyruvate (PEP).

In a preferred embodiment, the cell comprises a pathway for production of a sialylated di- and/or oligosaccharide comprising a pathway for production of ManNAc. ManNAc can be provided by an enzyme expressed in the cell or by the mechanism of the cell. Such cell producing ManNAc can express an N-acylglucosamine 2-epimerase like is known e.g. from several species including Bacteroides ovatus, E. coli, Homo sapiens, Rattus norvegicus that converts GlcNAc into ManNAc. Alternatively, and/or additionally, the cell producing ManNAc can express a UDP-N-acetylglucosamine 2-epimerase like is known e.g. from several species including Campylobacter jejuni, E. coli, Neisseria meningitidis, Bacillus subtilis, Citrobacter rodentium that converts UDP-GlcNAc into ManNAc. GlcNAc and/or UDP-GlcNAc can be added to the cell and/or provided by an enzyme expressed in the cell or by the mechanism of the cell as described herein.

In a more preferred embodiment, the cell is modified for enhanced ManNAc production. Said modification can be any one or more chosen from the group comprising knock-out of N-acetylmannosamine kinase, over-expression of N-acetylneuraminate lyase.

In another preferred embodiment, the cell comprises a pathway for production of a sialylated di- and/or oligosaccharide comprising a pathway for production of ManNAc-6-phosphate. ManNAc-6-phosphate can be provided by an enzyme expressed in the cell or by the mechanism of the cell. Such cell producing ManNAc-6-phosphate can express a bifunctional UDP-GlcNAc 2-epimerase/kinase like is known e.g. from several species including Homo sapiens, Rattus norvegicus and Mus musculus that converts UDP-GlcNAc into ManNAc-6-phosphate. Alternatively, and/or additionally, the cell producing ManNAc-6-phosphate can express an N-acetylmannosamine-6-phosphate 2-epimerase that converts GlcNAc-6-phosphate into ManNAc-6-phosphate. UDP-GlcNAc and/or GlcNAc-6-phosphate can be added to the cell and/or provided by an enzyme expressed in the cell or by the mechanism of the cell as described herein. In a more preferred embodiment, the cell is modified for enhanced ManNAc-6-phosphate production. Said modification can be any one or more chosen from the group comprising over-expression of N-acetylglucosamine-6-phosphate deacetylase, over-expression of N-acetyl-D-glucosamine kinase, over-expression of phosphoglucosamine mutase, over-expression of N-acetylglucosamine-1-phosphate uridyltransferase/glucosamine-1-phosphate acetyltransferase.

In another preferred embodiment, the cell comprises a pathway for production of a sialylated di- and/or oligosaccharide comprising a pathway for production of phosphoenolpyruvate (PEP).

In another preferred embodiment, the cell comprises a pathway for production of a sialylated di- and/or oligosaccharide comprising any one or more modifications for enhanced production and/or supply of PEP. In a preferred embodiment and as a means for enhanced production and/or supply of PEP, one or more PEP-dependent, sugar-transporting phosphotransferase system(s) is/are disrupted such as but not limited to: 1) the N-acetyl-D-glucosamine Npi-phosphotransferase (EC 2.7.1.193), which is for instance encoded by the nagE gene (or the cluster nagABCD) in E. coli or Bacillus species, 2) ManXYZ which encodes the Enzyme II Man complex (mannose PTS permease, protein-Npi-phosphohistidine-D-mannose phosphotransferase) that imports exogenous hexoses (mannose, glucose, glucosamine, fructose, 2-deoxyglucose, mannosamine, N-acetylglucosamine, etc.) and releases the phosphate esters into the cell cytoplasm, 3) the glucose-specific PTS transporter (for instance encoded by PtsG/Crr) which takes up glucose and forms glucose-6-phosphate in the cytoplasm, 4) the sucrose-specific PTS transporter which takes up sucrose and forms sucrose-6-phosphate in the cytoplasm, 5) the fructose-specific PTS transporter (for instance encoded by the genes fruA and fruB and the kinase fruK which takes up fructose and forms in a first step fructose-1-phosphate and in a second step fructose1,6 bisphosphate, 6) the lactose PTS transporter (for instance encoded by lacE in Lactococcus casei) which takes up lactose and forms lactose-6-phosphate, 7) the galactitol-specific PTS enzyme which takes up galactitol and/or sorbitol and forms galactitol-1-phosphate or sorbitol-6-phosphate respectively, 8) the mannitol-specific PTS enzyme which takes up mannitol and/or sorbitol and forms mannitol-1-phosphate or sorbitol-6-phosphate respectively, and 9) the trehalose-specific PTS enzyme which takes up trehalose and forms trehalose-6-phosphate.

In another and/or additional preferred embodiment and as a means for enhanced production and/or supply of PEP, the full PTS system is disrupted by disrupting the PtsIH/Crr gene cluster. PtsI (Enzyme I) is a cytoplasmic protein that serves as the gateway for the phosphoenolpyruvate:sugar phosphotransferase system (PTS^{sugar)} of E. coli K-12. PtsI is one of two (PtsI and PtsH) sugar non-specific protein constituents of the PTS^{sugar} which along with a sugar-specific inner membrane permease effects a phosphotransfer cascade that results in the coupled phosphorylation and transport of a variety of carbohydrate substrates. HPr (histidine containing protein) is one of two sugar-non-specific protein constituents of the PTS^{sugar}. It accepts a phosphoryl group from phosphorylated Enzyme I (PtsI-P) and then transfers it to the EIIA domain of any one of the many sugar-specific enzymes (collectively known as Enzymes II) of the PTS^{sugar}. Crr or EIIA^{Glc} is phosphorylated by PEP in a reaction requiring PtsH and PtsI.

In another and/or additional preferred embodiment, the cell is further modified to compensate for the deletion of a PTS system of a carbon source by the introduction and/or overexpression of the corresponding permease. These are e.g. permeases or ABC transporters that comprise but are not limited to transporters that specifically import lactose such as e.g. the transporter encoded by the LacYgene from E. coli, sucrose such as e.g. the transporter encoded by the cscB gene from E. coli, glucose such as e.g. the transporter encoded by the galP gene from E. coli, fructose such as e.g. the transporter encoded by the fruI gene from Streptococcus mutans, or the Sorbitol/mannitol ABC transporter such as the transporter encoded by the cluster SmoEFGK of Rhodobacter sphaeroides, the trehalose/sucrose/maltose transporter such as the transporter encoded by the gene cluster ThuEFGK of Sinorhizobium meliloti and the N-acetylglucosamine/galactose/glucose transporter such as the transporter encoded by NagP of Shewanella oneidensis. Examples of combinations of PTS deletions with overexpression of alternative transporters are: 1) the deletion of the glucose PTS system, e.g. ptsG gene, combined with the introduction and/or overexpression of a glucose permease (e.g. galP of glcP), 2) the deletion of the fructose PTS system, e.g. one or more of the fruB, fruA, fruK genes, combined with the introduction and/or overexpression of fructose permease, e.g. fruI, 3) the deletion of the lactose PTS system, combined with the introduction and/or overexpression of lactose permease, e.g. LacY, and/or 4) the deletion of the sucrose PTS system, combined with the introduction and/or overexpression of a sucrose permease, e.g. cscB.

In a further preferred embodiment, the cell is modified to compensate for the deletion of a PTS system of a carbon source by the introduction of carbohydrate kinases, such as glucokinase (EC 2.7.1.1, EC 2.7.1.2, EC 2.7.1.63), galactokinase (EC 2.7.1.6), and/or fructokinase (EC 2.7.1.3, EC 2.7.1.4). Examples of combinations of PTS deletions with overexpression of alternative transporters and a kinase are: 1) the deletion of the glucose PTS system, e.g. ptsG gene, combined with the introduction and/or overexpression of a glucose permease (e.g. galP of glcP), combined with the introduction and/or overexpression of a glucokinase (e.g. glk), and/or 2) the deletion of the fructose PTS system, e.g. one or more of the fruB, fruA, fruK genes, combined with the introduction and/or overexpression of fructose permease, e.g. fruI, combined with the introduction and/or overexpression of a fructokinase (e.g. frk or mak).

In another and/or additional preferred embodiment and as a means for enhanced production and/or supply of PEP, the cell is modified by the introduction of or modification in any one or more of the list comprising phosphoenolpyruvate synthase activity (EC: 2.7.9.2 encoded for instance in E. coli by ppsA), phosphoenolpyruvate carboxykinase activity (EC 4.1.1.32 or EC 4.1.1.49 encoded for instance in Corynebacterium glutamicum by PCK or in E. coli by pckA, resp.), phosphoenolpyruvate carboxylase activity (EC 4.1.1.31 encoded for instance in E. coli by ppc), oxaloacetate decarboxylase activity (EC 4.1.1.112 encoded for instance in E. coli by eda), pyruvate kinase activity (EC 2.7.1.40 encoded for instance in E. coli by pykA and pykF), pyruvate carboxylase activity (EC 6.4.1.1 encoded for instance in B. subtilis by pyc) and malate dehydrogenase activity (EC 1.1.1.38 or EC 1.1.1.40 encoded for instance in E. coli by maeA or maeB, resp.).

In a more preferred embodiment, the cell is modified to overexpress any one or more of the polypeptides comprising ppsA from E. coli (UniProt ID P23538), PCK from C. glutamicum (UniProt ID Q6F5A5), pcka from E. coli (UniProt ID P22259), eda from E. coli (UniProt ID P0A955), maeA from E. coli (UniProt ID P26616) and maeB from E. coli (UniProt ID P76558).

In another and/or additional preferred embodiment, the cell is modified to express any one or more of a functional homolog, variant or derivative of any one of UniProt IDs P23538, Q6F5A5, P22259, P0A955, P26616 or P76558 having at least 80% overall sequence identity to the full-length of any one of said polypeptide with UniProt IDs P23538, Q6F5A5, P22259, P0A955, P26616 or P76558, and having phosphoenolpyruvate synthase activity, phosphoenolpyruvate carboxykinase activity, oxaloacetate decarboxylase activity, or malate dehydrogenase activity, respectively.

In another and/or additional preferred embodiment and as a means for enhanced production and/or supply of PEP, the cell is modified by a reduced activity of phosphoenolpyruvate carboxylase activity, and/or pyruvate kinase activity, preferably a deletion of the genes encoding for phosphoenolpyruvate carboxylase, the pyruvate carboxylase activity and/or pyruvate kinase.

In an exemplary embodiment, the cell is genetically engineered by different adaptations such as the overexpression of phosphoenolpyruvate synthase combined with the deletion of a pyruvate kinase gene, the overexpression of phosphoenolpyruvate synthase combined with the deletion of a phosphoenolpyruvate carboxylase gene, the overexpression of phosphoenolpyruvate synthase combined with the deletion of a pyruvate carboxylase gene, the overexpression of phosphoenolpyruvate carboxykinase combined with the deletion of a pyruvate kinase gene, the overexpression of phosphoenolpyruvate carboxykinase combined with the deletion of a phosphoenolpyruvate carboxylase gene, the overexpression of phosphoenolpyruvate carboxykinase combined with the deletion of a pyruvate carboxylase gene, the overexpression of oxaloacetate decarboxylase combined with the deletion of a pyruvate kinase gene, the overexpression of oxaloacetate decarboxylase combined with the deletion of a phosphoenolpyruvate carboxylase gene, the overexpression of oxaloacetate decarboxylase combined with the deletion of a pyruvate carboxylase gene, the overexpression of malate dehydrogenase combined with the deletion of a pyruvate kinase gene, the overexpression of malate dehydrogenase combined with the deletion of a phosphoenolpyruvate carboxylase gene and/or the overexpression of malate dehydrogenase combined with the deletion of a pyruvate carboxylase gene.

In another exemplary embodiment, the cell is genetically engineered by different adaptations such as the overexpression of phosphoenolpyruvate synthase combined with the overexpression of a phosphoenolpyruvate carboxykinase, the overexpression of phosphoenolpyruvate synthase combined with the overexpression of an oxaloacetate decarboxylase, the overexpression of phosphoenolpyruvate synthase combined with the overexpression of a malate dehydrogenase, the overexpression of phosphoenolpyruvate carboxykinase combined with the overexpression of an oxaloacetate decarboxylase, the overexpression of phosphoenolpyruvate carboxykinase combined with the overexpression of a malate dehydrogenase, the overexpression of oxaloacetate decarboxylase combined with the overexpression of a malate dehydrogenase, the overexpression of phosphoenolpyruvate synthase combined with the overexpression of a phosphoenolpyruvate carboxykinase and the overexpression of an oxaloacetate decarboxylase, the overexpression of phosphoenolpyruvate synthase combined with the overexpression of a phosphoenolpyruvate carboxykinase and the overexpression of a malate dehydrogenase, the overexpression of phosphoenolpyruvate synthase combined with the overexpression of a phosphoenolpyruvate carboxykinase and the overexpression of an oxaloacetate decarboxylase and the overexpression of a malate dehydrogenase, the overexpression of phosphoenolpyruvate carboxykinase combined with the overexpression of an oxaloacetate decarboxylase and the overexpression of a malate dehydrogenase and/or the overexpression of phosphoenolpyruvate synthase combined with the overexpression of an oxaloacetate decarboxylase and the overexpression of a malate dehydrogenase.

In another exemplary embodiment, the cell is genetically engineered by different adaptations such as the overexpression of phosphoenolpyruvate synthase combined with the overexpression of a phosphoenolpyruvate carboxykinase combined with the deletion of a pyruvate kinase gene, the overexpression of phosphoenolpyruvate synthase combined with the overexpression of an oxaloacetate decarboxylase combined with the deletion of a pyruvate kinase gene, the overexpression of phosphoenolpyruvate synthase combined with the overexpression of a malate dehydrogenase combined with the deletion of a pyruvate kinase gene, the overexpression of phosphoenolpyruvate carboxykinase combined with the overexpression of an oxaloacetate decarboxylase combined with the deletion of a pyruvate kinase gene, the overexpression of phosphoenolpyruvate carboxykinase combined with the overexpression of a malate dehydrogenase combined with the deletion of a pyruvate kinase gene, the overexpression of an oxaloacetate decarboxylase combined with the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene, the overexpression of phosphoenolpyruvate synthase combined with the overexpression of a phosphoenolpyruvate carboxykinase and the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene, the overexpression of a phosphoenolpyruvate carboxykinase combined with the overexpression of an oxaloacetate decarboxylase and the overexpression of a malate dehydrogenase combined with the deletion of a pyruvate kinase gene, the overexpression of phosphoenolpyruvate synthase combined the overexpression of oxaloacetate decarboxylase and the overexpression of a malate dehydrogenase combined with the deletion of a pyruvate kinase gene.

In another exemplary embodiment, the cell is genetically engineered by different adaptations such as the overexpression of phosphoenolpyruvate synthase combined with the overexpression of a phosphoenolpyruvate carboxykinase combined with the deletion of a phosphoenolpyruvate carboxylase gene, the overexpression of phosphoenolpyruvate synthase combined with the overexpression of an oxaloacetate decarboxylase combined with the deletion of a phosphoenolpyruvate carboxylase gene, the overexpression of phosphoenolpyruvate synthase combined with the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase combined with the deletion of a phosphoenolpyruvate carboxylase gene, the overexpression of a phosphoenolpyruvate carboxykinase combined with the overexpression of a malate dehydrogenase combined with the deletion of a phosphoenolpyruvate carboxylase gene, the overexpression of an oxaloacetate decarboxylase combined with the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a phosphoenolpyruvate carboxylase gene, the overexpression of phosphoenolpyruvate synthase combined the overexpression of an oxaloacetate decarboxylase and the overexpression of a malate dehydrogenase combined with the deletion of a phosphoenolpyruvate carboxylase gene.

In another exemplary embodiment, the cell is genetically engineered by different adaptations such as the overexpression of phosphoenolpyruvate synthase combined with the overexpression of a phosphoenolpyruvate carboxykinase combined with the deletion of a pyruvate carboxylase gene, the overexpression of phosphoenolpyruvate synthase combined with the overexpression of an oxaloacetate decarboxylase combined with the deletion of a pyruvate carboxylase gene, the overexpression of phosphoenolpyruvate synthase combined with the overexpression of a malate dehydrogenase combined with the deletion of a pyruvate carboxylase gene, the overexpression of a phosphoenolpyruvate carboxykinase combined with the overexpression of an oxaloacetate decarboxylase combined with the deletion of a pyruvate carboxylase gene, the overexpression of a phosphoenolpyruvate carboxykinase combined with the overexpression of a malate dehydrogenase combined with the deletion of a pyruvate carboxylase gene, the overexpression of an oxaloacetate decarboxylase combined with the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate carboxylase gene, the overexpression of phosphoenolpyruvate synthase combined with the overexpression of an oxaloacetate decarboxylase and the overexpression of a malate dehydrogenase combined with the deletion of a pyruvate carboxylase gene.

In another exemplary embodiment, the cell is genetically engineered by different adaptations such as the overexpression of phosphoenolpyruvate synthase combined with the overexpression of a phosphoenolpyruvate carboxykinase combined with the deletion of a pyruvate kinase gene and a phosphoenolpyruvate carboxylase gene, the overexpression of phosphoenolpyruvate synthase combined with the overexpression of an oxaloacetate decarboxylase combined with the deletion of a pyruvate kinase gene and a phosphoenolpyruvate carboxylase gene, the overexpression of phosphoenolpyruvate synthase combined with the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a phosphoenolpyruvate carboxylase gene, the overexpression of a phosphoenolpyruvate carboxykinase combined with the overexpression of a malate dehydrogenase combined with the deletion of a pyruvate kinase gene and a phosphoenolpyruvate carboxylase gene, the overexpression of an oxaloacetate decarboxylase combined with the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate synthase combined with the overexpression of an oxaloacetate decarboxylase and the overexpression of a malate dehydrogenase combined with the deletion of a pyruvate kinase gene and a phosphoenolpyruvate carboxylase gene.

In another exemplary embodiment, the cell is genetically engineered by different adaptations such as the overexpression of phosphoenolpyruvate synthase combined with the overexpression of a phosphoenolpyruvate carboxykinase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a malate dehydrogenase combined with the deletion of a pyruvate kinase gene and a pyruvate carboxylase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a pyruvate carboxylase gene and a phosphoenolpyruvate carboxylase gene, the overexpression of a phosphoenolpyruvate carboxykinase combined with the overexpression of a malate dehydrogenase combined with the deletion of a pyruvate kinase gene and a pyruvate carboxylase gene and a phosphoenolpyruvate carboxylase gene, the overexpression of an oxaloacetate decarboxylase combined with the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and pyruvate carboxylase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene. According to another aspect of the method and/or cell of the invention, the cell is further capable to synthesize any one or more nucleotide-activated sugars. In a preferred embodiment of the method and/or cell of the invention, the cell is capable to synthesize one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-fucose (GDP-Fuc), GDP-rhamnose and UDP-xylose. In a more preferred embodiment of the method and/or cell of the invention, the nucleotide-activated sugar is synthesized in the cell by at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine epimerase, UDP-N-acetylglucosamine 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, N-acetyl mannosamine-6-phosphate 2-epimerase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphatase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-GalNAc pyrophosphorylase.

The host cell used herein is optionally genetically engineered to express the de novo synthesis of UDP-GlcNAc. UDP-GlcNAc can be provided by an enzyme expressed in the cell or by the metabolism of the cell. Such cell producing a UDP-GlcNAc can express enzymes converting, e.g. GlcNAc, which is to be added to the cell, to UDP-GlcNAc. These enzymes may be any one or more of the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the host cell used herein is optionally genetically engineered to express the de novo synthesis of CMP-Neu5Ac. CMP-Neu5Ac can be provided by an enzyme expressed in the cell or by the metabolism of the cell. Such cell producing CMP-Neu5Ac can express an enzyme converting, e.g., sialic acid to CMP-Neu5Ac. This enzyme may be a CMP-sialic acid synthase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. Said modification can be any one or more chosen from the group comprising knock-out of an N-acetylglucosamine-6-phosphate deacetylase, knock-out of a glucosamine-6-phosphate deaminase, over-expression of a CMP-sialic acid synthase, and over-expression of an N-acyl-D-glucosamine 2-epimerase encoding gene.

Additionally, or alternatively, the host cell used herein is optionally genetically engineered to express the de novo synthesis of CMP-Neu5Gc. CMP-Neu5Gc can be synthesized directly from CMP-Neu5Ac via a hydroxylation reaction performed by a vertebrate CMP-Neu5Ac hydroxylase (CMAH) enzyme. Preferably, the cell is modified to produce CMP-Neu5Gc. More preferably, the cell is modified for enhanced CMP-Neu5Gc production.

Additionally, or alternatively, the host cell used herein is optionally genetically engineered to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. Said modification can be any one or more chosen from the group comprising knock-out of a UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the host cell used herein is optionally genetically engineered to express the de novo synthesis of UDP-Gal. UDP-Gal can be provided by an enzyme expressed in the cell or by the metabolism of the cell. Such cell producing UDP-Gal can express an enzyme converting, e.g. UDP-glucose, to UDP-Gal. This enzyme may be, e.g., the UDP-glucose 4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. Said modification can be any one or more chosen from the group comprising knock-out of a bifunctional 5′-nucleotidase/UDP-sugar hydrolase encoding gene, knock-out of a galactose-1-phosphate uridylyltransferase encoding gene and over-expression of a UDP-glucose 4-epimerase encoding gene.

Additionally, or alternatively, the host cell used herein is optionally genetically engineered to express the de novo synthesis of UDP-GalNAc. UDP-GalNAc can be synthesized from UDP-GlcNAc by the action of a single-step reaction using a UDP-N-acetylglucosamine 4-epimerase like e.g. wbgU from Plesiomonas shigelloides, gne from Yersinia enterocolitica or wbpP from Pseudomonas aeruginosa serotype 06. Preferably, the cell is modified to produce UDP-GalNAc. More preferably, the cell is modified for enhanced UDP-GalNAc production.

Additionally, or alternatively, the host cell used herein is optionally genetically engineered to express the de novo synthesis of UDP-ManNAc. UDP-ManNAc can be synthesized directly from UDP-GlcNAc via an epimerization reaction performed by a UDP-GlcNAc 2-epimerase (like e.g. cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

According to another aspect of the method and/or cell of the invention, the cell expresses at least one glycosyltransferase chosen from the list comprising fucosyltransferases, galactosyltransferases, glucosyltransferases, mannosyltransferases, N-acetylglucosaminyltransferases, N-acetylgalactosaminyltransferases, N-acetyl mannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a preferred embodiment of the method and/or cell of the invention, the fucosyltransferase is chosen from the list comprising alpha-1,2-fucosyltransferase, alpha-1,3-fucosyltransferase, alpha1-,3/4-fucosyltransferase, alpha-1,4-fucosyltransferase and alpha-1,6-fucosyltransferase.

In an alternative and/or additional embodiment of the method and/or cell of the invention, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.

In an alternative and/or additional embodiment of the method and/or cell of the invention, the glucosyltransferase is chosen from the list comprising alpha-glucosyltransferase, beta-1,2-glucosyltransferase, beta-1,3-glucosyltransferase and beta-1,4-glucosyltransferase.

In an alternative and/or additional embodiment of the method and/or cell of the invention, the mannosyltransferase is chosen from the list comprising alpha-1,2-mannosyltransferase, alpha-1,3-mannosyltransferase and alpha-1,6-mannosyltransferase.

In an alternative and/or additional embodiment of the method and/or cell of the invention, the N acetylglucosaminyltransferase is chosen from the list comprising galactoside beta-1,3-N-acetylglucosaminyltransferase and beta-1,6-N-acetylglucosaminyltransferase.

In an alternative and/or additional embodiment of the method and/or cell of the invention, the N-acetylgalactosaminyltransferase is chosen from the list comprising alpha-1,3-N-acetylgalactosaminyltransferase.

In a further aspect of the method and/or cell of the invention, the cell is modified in the expression or activity of at least one of said glycosyltransferases. In a preferred embodiment, said glycosyltransferase is an endogenous protein of the cell with a modified expression or activity, preferably said endogenous glycosyltransferase is overexpressed; alternatively said glycosyltransferase is a heterologous protein that is heterogeneously introduced and expressed in said cell, preferably overexpressed. Said endogenous glycosyltransferase can have a modified expression in the cell which also expresses a heterologous glycosyltransferase.

According to another and/or alternative preferred aspect of the method and/or cell of the invention, the cell comprises a fucosylation pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-fucokinase/GDP-fucose pyrophosphorylase, fucosyltransferase.

According to another and/or alternative preferred aspect of the method and/or cell of the invention, the cell comprises a galactosylation pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase, galactosyltransferase.

According to another and/or alternative preferred aspect of the method and/or cell of the invention, the cell comprises an N-acetylglucosaminylation pathway comprising at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridyltransferase/glucosamine-1-phosphate acetyltransferase, N-acetylglucosaminyltransferase.

According to another preferred aspect of the method and/or cell of the invention, the cell comprises a modification for reduced production of acetate. Said modification can be any one or more chosen from the group comprising overexpression of an acetyl-coenzyme A synthetase, a full or partial knock-out or rendered less functional pyruvate dehydrogenase and a full or partial knock-out or rendered less functional lactate dehydrogenase.

In a further aspect of the method and/or cell of the invention, the cell is modified in the expression or activity of at least one acetyl-coenzyme A synthetase like e.g. acs from E. coli, S. cerevisiae, H. sapiens, M. musculus. In a preferred embodiment, said acetyl-coenzyme A synthetase is an endogenous protein of the cell with a modified expression or activity, preferably said endogenous acetyl-coenzyme A synthetase is overexpressed; alternatively, said acetyl-coenzyme A synthetase is a heterologous protein that is heterogeneously introduced and expressed in said cell, preferably overexpressed. Said endogenous acetyl-coenzyme A synthetase can have a modified expression in the cell which also expresses a heterologous can have a modified expression in the cell which also expresses a heterologous. In a more preferred embodiment, the cell is modified in the expression or activity of the acetyl-coenzyme A synthetase acs from E. coli (UniProt ID P27550). In another and/or additional preferred embodiment, the cell is modified in the expression or activity of a functional homolog, variant or derivative of UniProt ID P27550 having at least 80% overall sequence identity to the full-length of said polypeptide with UniProt ID P27550 and having acetyl-coenzyme A synthetase activity.

In an alternative and/or additional further aspect of the method and/or cell of the invention, the cell is modified in the expression or activity of at least one pyruvate dehydrogenase like e.g. from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity.

In an alternative and/or additional further aspect of the method and/or cell of the invention, the cell is modified in the expression or activity of at least one lactate dehydrogenase like e.g. from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the IdhA encoding gene resulting in a cell lacking lactate dehydrogenase activity.

According to another preferred aspect of the method and/or cell of the invention, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IcIR, Ion protease, glucose-specific translocating phosphotransferase enzyme IIBC component ptsG, glucose-specific translocating phosphotransferase (PTS) enzyme IIBC component maIX, enzyme IIA, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

According to another preferred aspect of the method and/or cell of the invention, the cell comprises a catabolic pathway for selected mono-, di- or oligosaccharides which is at least partially inactivated, the mono-, di-, or oligosaccharides being involved in and/or required for the synthesis of a sialylated di- and/or oligosaccharide.

According to another preferred aspect of the method and/or cell of the invention, the cell is using a precursor for the synthesis of said sialylated di- and/or oligosaccharide. Herein, the precursor is fed to the cell from the cultivation medium. In another preferred embodiment, the cell is producing a precursor for the synthesis of said sialylated di- and/or oligosaccharide.

In another preferred aspect of the method of present invention, the cell uses at least one precursor for the synthesis of a sialylated di- and/or oligosaccharide of present invention. In a more preferred embodiment, the cell uses two or more precursors for the synthesis of a sialylated di- and/or oligosaccharide of present invention.

According to another preferred aspect of the method and/or cell of the invention, the cell produces 90 g/L or more of a sialylated di- and/or oligosaccharide in the whole broth and/or supernatant. In a more preferred embodiment, the sialylated di- and/or oligosaccharide produced in the whole broth and/or supernatant has a purity of at least 80% measured on the total amount of sialylated di- and/or oligosaccharide and its precursor produced by the cell in the whole broth and/or supernatant, respectively.

According to another aspect of the method and/or cell of the invention, the sialylated di- and/or oligosaccharide is chosen from the list comprising a milk oligosaccharide, O-antigen, the oligosaccharide repeats present in capsular polysaccharides, peptidoglycan, amino-sugars and Lewis-type antigen oligosaccharides. In a more preferred embodiment, the milk oligosaccharide is a mammalian milk oligosaccharide. In an even more preferred embodiment, the milk oligosaccharide is a human milk oligosaccharide.

Another aspect of the invention provides for a method and a cell wherein a sialylated di- and/or oligosaccharide is produced in and/or by a microorganism chosen from the list consisting of a bacterium, fungus or yeast. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably the present invention specifically relates to a mutated and/or transformed Escherichia coli strain as indicated above wherein said E. coli strain is a K12 strain. More specifically, the present invention relates to a mutated and/or transformed Escherichia coli strain as indicated above wherein said K12 strain is E. coli MG1655. The latter bacterium belonging to the phylum Firmicutes belongs preferably to the Bacilli, preferably from the species Bacillus. The latter fungus belongs preferably to the genus Rhizopus, Dictyostelium, Penicillium, Mucor or Aspergillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Zygosaccharomyces, Pichia, Komagataella, Hansenula, Yarrowia, Starmerella, Kluyveromyces or Debaromyces.

Another aspect provides for a cell to be stably cultured in a medium, wherein said medium can be any type of growth medium comprising minimal medium, complex medium or growth medium enriched in certain compounds like for example but not limited to vitamins, trace elements, amino acids.

The microorganism or cell as used herein is capable to grow on a monosaccharide, disaccharide, oligosaccharide, polysaccharide, polyol, glycerol, a complex medium or a mixture thereof like e.g. a mixed feedstock, preferably a mixed monosaccharide feedstock like e.g. hydrolysed sucrose as the main carbon source. With the term main is meant the most important carbon source for the microorganism or cell for the production of the sialylated di- and/or oligosaccharide of interest, biomass formation, carbon dioxide and/or by-products formation (such as acids and/or alcohols, such as acetate, lactate, and/or ethanol), i.e. 20, 30, 40, 50, 60, 70, 75, 80, 85, 90, 95, 98, 99% of all the required carbon is derived from the above-indicated carbon source. With the term complex medium is meant a medium for which the exact constitution is not determined. Examples are molasses, corn steep liquor, peptone, tryptone or yeast extract. In one embodiment of the invention, said carbon source is the sole carbon source for said organism, i.e. 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of the sialylated di- and/or oligosaccharide.

According to another aspect of the method and/or cell of the invention, the cell is capable to synthesize a mixture of oligosaccharides comprising at least one sialylated oligosaccharide. In an alternative and/or additional aspect, the cell is capable to synthesize a mixture of di- and oligosaccharides comprising at least one sialylated di- an/or oligosaccharide; alternatively, the cell is capable to synthesize a mixture of sialic acid, di- and/or oligosaccharides.

In a further preferred aspect, the method for the production of a sialylated di- and/or oligosaccharide as defined herein comprises at least one of the following steps:
i) Use of a culture medium comprising at least one precursor and/or acceptor for the production of said sialylated di- and/or oligosaccharide, and/or
ii) Adding to the culture medium at least one precursor and/or acceptor feed for the production of said sialylated di- and/or oligosaccharide.

In another and/or additional further preferred aspect, the method for the production of a sialylated di- and/or oligosaccharide as described herein comprises at least one of the following steps:
i) Use of a culture medium comprising at least one precursor and/or acceptor;
ii) Adding to the culture medium in a reactor at least one precursor and/or acceptor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed;
iii) Adding to the culture medium in a reactor at least one precursor and/or acceptor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed and wherein preferably, the pH of said precursor and/or acceptor feed is set between 2.0 and 10.0 and wherein preferably, the temperature of said precursor and/or acceptor feed is kept between 20° C. and 80° C.;
iv) Adding at least one precursor and/or acceptor feed in a continuous manner to the culture medium over the course of 1 day, 2 days, 3 days, 4 days, 5 days by means of a feeding solution;
v) Adding at least one precursor and/or acceptor feed in a continuous manner to the culture medium over the course of 1 day, 2 days, 3 days, 4 days, 5 days by means of a feeding solution and wherein preferably, the pH of said precursor and/or acceptor feed is set between 2.0 and 10.0, preferably between 3 and 7, and wherein preferably, the temperature of said precursor and/or acceptor feed is kept between 20° C. and 80° C.;

said method resulting in a sialylated di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In another and/or additional further preferred aspect, the method for the production of a sialylated di- and/or oligosaccharide as described herein comprises at least one of the following steps:
i) Use of a culture medium comprising at least one precursor and/or acceptor;
ii) Adding to the culture medium in a reactor at least one precursor and/or acceptor in one pulse or in a discontinuous (pulsed) manner wherein the total reactor volume ranges from 250 mL (millilitre) to 10.000 m³ (cubic meter), preferably so that the final volume of the culture medium is not more than three-fold, preferably not more than two-fold, more preferably less than 2-fold of the volume of the culture medium before the addition of said precursor and/or acceptor feed pulse(s);
iii) Adding to the culture medium in a reactor at least one precursor and/or acceptor in one pulse or in a discontinuous (pulsed) manner wherein the total reactor volume ranges from 250 mL (millilitre) to 10.000 m³ (cubic meter), preferably so that the final volume of the culture medium is not more than three-fold, preferably not more than two-fold, more preferably less than 2-fold of the volume of the culture medium before the addition of said precursor and/or acceptor feed pulse(s) and wherein preferably, the pH of said feed pulse(s) is set between 2.0 and 10.0, preferably between 3 and 7, and wherein preferably, the temperature of said feed pulse(s) is kept between 20° C. and 80° C.;
iv) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
v) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of said precursor and/or acceptor feed is set between 2.0 and 10.0, preferably between 3 and 7, and wherein preferably, the temperature of said precursor and/or acceptor feed is kept between 20° C. and 80° C.;

said method resulting in a sialylated di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In another and/or additional further preferred aspect, the method for the production of a sialylated di- and/or oligosaccharide as described herein comprises at least one of the following steps:
i) Use of a culture medium comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter);
ii) Use of a culture medium comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter);
iii) Adding to the culture medium in a reactor a precursor feed comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter), preferably in a continuous manner, and preferably so that the final volume of the culture medium is not more than three fold, preferably not more than two-fold, more preferably less than 2-fold of the volume of the culture medium before the addition of said precursor feed;
iv) Adding to the culture medium in a reactor an acceptor feed comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said acceptor feed;
v) Adding to the culture medium in a reactor a precursor feed comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter), preferably in a continuous manner, and preferably so that the final volume of the culture medium is not more than three fold, preferably not more than two-fold, more preferably less than 2-fold of the volume of the culture medium before the addition of said precursor feed and wherein preferably, the pH of said precursor feed is set between 2.0 and 10.0 and wherein preferably, the temperature of said precursor feed is kept between 20° C. and 80° C.;
vi) Adding to the culture medium in a reactor an acceptor feed comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter), preferably in a continuous manner, and preferably so that the final volume of the culture medium is not more than three fold, preferably not more than two-fold, more preferably less than 2-fold of the volume of the culture medium before the addition of said acceptor feed and wherein preferably, the pH of said acceptor feed is set between 2.0 and 10.0 and wherein preferably, the temperature of said acceptor feed is kept between 20° C. and 80° C.;
vii) Adding a precursor and/or acceptor feed in a continuous manner to the culture medium over the course of 1 day, 2 days, 3 days, 4 days, 5 days by means of a feeding solution;
viii) Adding a precursor feed in a continuous manner to the culture medium over the course of 1 day, 2 days, 3 days, 4 days, 5 days by means of a precursor feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said precursor feeding solution is set between 2.0 and 10.0 and wherein preferably, the temperature of said precursor feeding solution is kept between 20° C. and 80° C.;
ix) Adding an acceptor feed in a continuous manner to the culture medium over the course of 1 day, 2 days, 3 days, 4 days, 5 days by means of an acceptor feeding solution and wherein the concentration of said acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said acceptor feeding solution is set between 2.0 and 10.0 and wherein preferably, the temperature of said acceptor feeding solution is kept between 20° C. and 80° C.;
said method resulting in a sialylated di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In a further, more preferred aspect, the method for the production of a sialylated di- and/or oligosaccharide as described herein comprises at least one of the following steps:
i) Use of a culture medium comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter);
ii) Use of a culture medium comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter);
iii) Adding to the culture medium in a reactor at least one precursor in one pulse or in a discontinuous (pulsed) manner comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter), and preferably so that the final volume of the culture medium is not more than three-fold, preferably not more than two-fold, more preferably less than 2-fold of the volume of the culture medium before the addition of said precursor feed pulse(s); iv) Adding to the culture medium in a reactor at least one acceptor in one pulse or in a discontinuous (pulsed) manner comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter), and preferably so that the final volume of the culture medium is not more than three fold, preferably not more than two-fold, more preferably less than 2-fold of the volume of the culture medium before the addition of said acceptor feed pulse(s);
v) Adding to the culture medium in a reactor at least one precursor in one pulse or in a discontinuous (pulsed) manner comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter), and preferably so that the final volume of the culture medium is not more than three-fold, preferably not more than two-fold, more preferably less than 2-fold of the volume of the culture medium before the addition of said precursor feed pulse(s) and wherein preferably, the pH of said precursor feed pulse(s) is set between 2.0 and 10.0 and wherein preferably, the temperature of said precursor feed pulse(s) is kept between 20° C. and 80° C.;
vi) Adding to the culture medium in a reactor at least one acceptor in one pulse or in a discontinuous (pulsed) manner comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter), and preferably so that the final volume of the culture medium is not more than three-fold, preferably not more than two-fold, more preferably less than 2-fold of the volume of the culture medium before the addition of said acceptor feed pulse(s) and wherein preferably, the pH of said acceptor feed pulse(s) is set between 2.0 and 10.0 and wherein preferably, the temperature of said acceptor feed pulse(s) is kept between 20° C. and 80° C.;
vii) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a discontinuous (pulsed) feeding solution;
viii) Adding a precursor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a discontinuous (pulsed) precursor feeding solution and wherein the concentration of said discontinuous (pulsed) precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said discontinuous (pulsed) precursor feeding solution is set between 2.0 and 10.0 and wherein preferably, the temperature of said discontinuous (pulsed) precursor feeding solution is kept between 20° C. and 80° C.;
ix) Adding an acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a discontinuous (pulsed) acceptor feeding solution and wherein the concentration of said discontinuous (pulsed) acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said discontinuous (pulsed) acceptor feeding solution is set between 2.0 and 10.0 and wherein preferably, the temperature of said discontinuous (pulsed) acceptor feeding solution is kept between 20° C. and 80° C.;
said method resulting in a sialylated di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In a further, more preferred aspect, the method for the production of a sialylated di- and/or oligosaccharide as described herein comprises at least one of the following steps:
i) Use of a culture medium comprising at least 50, more preferably at least 75, more preferably at least 100, more preferably at least 120, more preferably at least 150 grams of lactose per litre of initial reactor volume wherein the total reactor volume ranges from 250 mL to 10.000 m³ (cubic meter);
ii) Adding to the culture medium in a reactor a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
iii) Adding to the culture medium in a reactor a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably, the pH of said lactose feed is set between 2.0 and 10.0, preferably between 3 and 7, and wherein preferably, the temperature of said lactose feed is kept between 20° C. and 80° C.;
iv) Adding a lactose feed in a continuous manner to the culture medium over the course of 1 day, 2 days, 3 days, 4 days, 5 days by means of a feeding solution;
v) Adding a lactose feed in a continuous manner to the culture medium over the course of 1 day, 2 days, 3 days, 4 days, 5 days by means of a lactose feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said lactose feeding solution is set between 2.0 and 10.0, preferably between 3 and 7, and wherein preferably the temperature of said lactose feeding solution is kept between 20° C. and 80° C.;

said method resulting in a sialylated oligosaccharide produced from said lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

Preferably the lactose feed is accomplished by adding lactose from the beginning of the cultivation in a concentration of at least 5 mM, preferably in a concentration of 30, 40, 50, 60, 70, 80, 90, 100, 150 mM, more preferably in a concentration >300 mM.

In another aspect the lactose feed is accomplished by adding lactose to the culture or cultivation medium in a concentration, such that throughout the production phase of the cultivation a lactose concentration of at least 5 mM, preferably 10 mM or 30 mM is obtained.

In a further embodiment of the methods described herein the host cells are cultivated for at least about 60, 80, 100, or about 120 hours or in a continuous manner.

In a preferred embodiment, a carbon and energy source, preferably glucose, glycerol, fructose, maltose, arabinose, maltodextrines, malto-oligosaccharides, maltotriose, sorbitol, xylose, rhamnose, sucrose, galactose, mannose, methanol, ethanol, trehalose, starch, cellulose, hemi-cellulose, polyols, corn-steep liquor, high-fructose syrup, succinate, malate, acetate, citrate, lactate and pyruvate, is also added, preferably continuously to the culture medium, preferably with the precursor and/or acceptor.

In another and/or additional preferred embodiment, a carbon source is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per litre of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon source, preferably glucose or sucrose, to the culture medium before the precursor and/or acceptor, preferably the lactose, is added to the culture medium in a second phase.

In an alternative preferable embodiment, in the method as described herein, precursor and/or acceptor, preferably the lactose, is added already in the first phase of exponential growth together with the carbon-based substrate.

In another preferred embodiment of the method of present invention, the culture medium contains at least one molecule selected from the group comprising lactose, galactose, sialic acid, fucose, GlcNAc, GalNAc, lacto-N-biose (LNB), N-acetyllactosamine (LacNAc).

According to the present invention, the methods as described herein preferably comprises a step of separating said sialylated di- and/or oligosaccharide from said cultivation.

The terms “separating from said cultivation” means harvesting, collecting, or retrieving said sialylated di- and/or oligosaccharide from the cell and/or the medium of its growth.

The sialylated di- and/or oligosaccharide can be separated in a conventional manner from the aqueous culture medium, in which the cell was grown. In case said sialylated di- and/or oligosaccharide is still present in the cells producing the sialylated di- and/or oligosaccharide, conventional manners to free or to extract said sialylated di- and/or oligosaccharide out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium and/or cell extract together and separately can then be further used for separating said sialylated di- and/or oligosaccharide.

This preferably involves clarifying said sialylated di- and/or oligosaccharide to remove suspended particulates and contaminants, particularly cells, cell components, insoluble metabolites and debris produced by culturing the genetically engineered cell. In this step, said sialylated di- and/or oligosaccharide can be clarified in a conventional manner. Preferably, said sialylated di- and/or oligosaccharide is clarified by centrifugation, flocculation, decantation and/or filtration. Another step of separating said sialylated di- and/or oligosaccharide preferably involves removing substantially all the proteins, peptides, amino acids, RNA, DNA, endotoxins and glycolipids that could interfere with the subsequent separation step, from said sialylated di- and/or oligosaccharide, preferably after it has been clarified. In this step, proteins and related impurities can be removed from said sialylated di- and/or oligosaccharide in a conventional manner. Preferably, proteins, salts, by-products, colour, endotoxins and other related impurities are removed from said sialylated di- and/or oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g. using slab-polyacrylamide or sodium dodecyl sulphate-polyacrylamide gel electrophoresis (PAGE)), affinity chromatography (using affinity ligands including e.g. DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography or electrodialysis. With the exception of size exclusion chromatography, proteins and related impurities are retained by a chromatography medium or a selected membrane.

In a further preferred embodiment, the methods as described herein also provide for a further purification of the sialylated di- and/or oligosaccharide of present invention. A further purification of said sialylated di- and/or oligosaccharide may be accomplished, for example, by use of (activated) charcoal or carbon, nanofiltration, ultrafiltration, electrophoresis, enzymatic treatment or ion exchange, temperature adjustment, pH adjustment or pH adjustment with an alkaline or acidic solution to remove any remaining DNA, protein, LPS, endotoxins, or other impurity. Alcohols, such as ethanol, and aqueous alcohol mixtures can also be used. Another purification step is accomplished by crystallization, evaporation or precipitation of said sialylated di- and/or oligosaccharide. Another purification step is to dry, e.g. spray dry, lyophilize, spray freeze dry, freeze spray dry, band dry, belt dry, vacuum band dry, vacuum belt dry, drum dry, roller dry, vacuum drum dry or vacuum roller dry the produced sialylated di- and/or oligosaccharide.

In an exemplary embodiment, the separation and purification of the sialylated di- and/or oligosaccharide is made in a process, comprising the following steps in any order:
a) contacting the cultivation or a clarified version thereof with a nanofiltration membrane with a molecular weight cut-off (MWCO) of 600-3500 Da ensuring the retention of the produced sialylated di- and/or oligosaccharide and allowing at least a part of the proteins, salts, by-products, colour and other related impurities to pass,
b) conducting a diafiltration process on the retentate from step a), using said membrane, with an aqueous solution of an inorganic electrolyte, followed by optional diafiltration with pure water to remove excess of the electrolyte,
c) and collecting the retentate enriched in said sialylated di- and/or oligosaccharide in the form of a salt from the cation of said electrolyte.

In an alternative exemplary embodiment, the separation and purification of said sialylated di- and/or oligosaccharide is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of said sialylated di- and/or oligosaccharide is made in a process, comprising treating the cultivation or a clarified version thereof with a strong cation exchange resin in H+-form in a step and with a weak anion exchange resin in free base form, in another step, wherein said steps can be performed in any order.

In an alternative exemplary embodiment, the separation and purification of said sialylated di- and/or oligosaccharide is made in the following way. The cultivation comprising the produced sialylated di- and/or oligosaccharide, biomass, medium components and contaminants, preferably wherein the purity of the produced sialylated di- and/or oligosaccharide in the cultivation is <80 percent, is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,

wherein a purified solution comprising the produced sialylated di- and/or oligosaccharide at a purity of greater than or equal to 80 percent is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In an alternative exemplary embodiment, the separation and purification of the sialylated di- and/or oligosaccharide is made in a process, comprising the following steps in any order: enzymatic treatment of the cultivation; removal of the biomass from the cultivation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, the present invention provides the produced sialylated di- and/or oligosaccharide which is dried to powder, wherein the dried powder contains <15 percent-wt. of water, preferably <10 percent-wt. of water, more preferably <7 percent-wt. of water, most preferably <5 percent-wt. of water.

In another specific embodiment, the present invention provides the produced sialylated di- and/or oligosaccharide which is spray-dried to powder, wherein the spray-dried powder contains <15 percent-wt. of water, preferably <10 percent-wt. of water, more preferably <7 percent-wt. of water, most preferably <5 percent-wt. of water.

Another aspect of the present invention provides the use of a sialic acid transporter selected from SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59 in the fermentative production of a sialylated di- and/or oligosaccharide. In another preferred embodiment, the present invention provides the use of a functional homolog, variant or derivative of any one of the polypeptides with SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59 having at least 80% overall sequence identity to the full-length sequence of any one of the polypeptides with SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59, respectively, and having sialic acid transporter activity in the fermentative production of a sialylated di- and/or oligosaccharide.

Another aspect of the present invention provides the use of a cell as defined herein, in a method for the production of a sialylated di- and/or oligosaccharide. A further aspect of the present invention provides the use of a method as defined herein for the production of a sialylated di- and/or oligosaccharide.

Furthermore, the invention also relates to the sialylated di- and/or oligosaccharide obtained by the methods according to the invention, as well as to the use of a polynucleotide, the vector, host cells, microorganisms or the polypeptide as described above for the production of said sialylated di- and/or oligosaccharide. Said sialylated di- and/or oligosaccharide may be used for the manufacture of a preparation, as food additive, prebiotic, symbiotic, for the supplementation of baby food, adult food, infant animal feed, adult animal feed, or as either therapeutically or pharmaceutically active compound or in cosmetic applications. With the novel methods, the sialylated di- and/or oligosaccharide can easily and effectively be provided, without the need for complicated, time and cost consuming synthetic processes.

For identification of the sialylated di- and/or oligosaccharide produced in the cell as described herein, the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the sialylated di- and/or oligosaccharide methods such as e.g. acid-catalysed hydrolysis, HPLC (high performance liquid chromatography) or GLC (gas-liquid chromatography) (after conversion to alditol acetates) may be used. To determine the glycosidic linkages, the sialylated di- and/or oligosaccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the sialylated di- and/or oligosaccharide is subjected to enzymatic analysis, e.g. it is contacted with an enzyme that is specific for a particular type of linkage, e.g., beta-galactosidase, or alpha-glucosidase, etc., and NMR may be used to analyse the products.

The separated and preferably also purified sialylated di- and/or oligosaccharide as described herein is incorporated into a food (e.g., human food or feed), dietary supplement, pharmaceutical ingredient, cosmetic ingredient or medicine. In some embodiments, the sialylated di- and/or oligosaccharide is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the sialylated di- and/or oligosaccharide being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, a sialylated di- and/or oligosaccharide produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavourings.

In some embodiments, the sialylated oligosaccharide is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, a sialylated oligosaccharide produced and/or purified by a process in this specification is included in infant formula to provide nutritional benefits similar to those provided by the oligosaccharides in human breast milk. In some embodiments, the sialylated oligosaccharide is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the sialylated oligosaccharide in the infant formula is approximately the same concentration as the concentration of the sialylated oligosaccharide generally present in human breast milk.

In some embodiments, the sialylated oligosaccharide is incorporated into a feed preparation, wherein said feed is chosen from the list comprising pet food, animal milk replacer, veterinary product, veterinary feed supplement, nutrition supplement, post weaning feed, or creep feed.

As will be shown in the examples herein, the newly identified sialic acid transporters have proven to be useful in the fermentative production of sialylated di- and/or oligosaccharides. The method and the cell of the invention preferably provide at least one of the following further surprising advantages when using sialic acid transporters as defined herein:
High and/or better titres of the sialylated di- and/or oligosaccharide (g/L),
High and/or better production rate r (g sialylated di- and/or oligosaccharide/L/h),
High and/or better cell performance index CPI (g sialylated di- and/or oligosaccharide/g X),
High and/or better specific productivity Up (g sialylated di- and/or oligosaccharide/g X/h),
High and/or better yield on sucrose Ys (g sialylated di- and/or oligosaccharide/g sucrose),
High and/or better sucrose uptake/conversion rate Qs (g sucrose/g X/h),
High and/or better lactose conversion/consumption rate rs (g lactose/h),
High and/or enhanced excretion or secretion of the sialylated di- and/or oligosaccharide, and/or
High and/or enhanced growth speed of the production host,

when compared to a production host for a sialylated di- and/or oligosaccharide with an identical genetic background but lacking the expression of the homologous and/or heterologous sialic acid transporter and/or (over)expression of the endogenous sialic acid transporter.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features which are still to be explained below can be used not only in the respectively specified combinations, but also in other combinations or on their own, without departing from the scope of the present invention.

Moreover, the present invention relates to the following specific embodiments:
1. A metabolically engineered cell of a microorganism for production of a sialylated di- and/or oligosaccharide, said cell comprising a pathway for production of said sialylated di- and/or oligosaccharide, characterized in that said cell:
   synthesizes sialic acid,
   expresses at least one sialyltransferase, preferably is modified in the expression or activity of at least one sialyltransferase,
   is modified to have a fully or partially knocked out or rendered less functional sialic acid catabolic pathway, and
   is modified for overexpression of an endogenous sialic acid transporter and/or expression of an exogenous, homologous and/or heterologous sialic acid transporter,
   preferably, said cell is modified in the expression or activity of any one of said sialyltransferase.
2. Cell according to embodiment 1, wherein said pathway for production of said sialylated di- and/or oligosaccharide comprises a sialylation pathway.
3. Cell according to embodiment 2, wherein said sialylation pathway comprises a sialyltransferase combined with at least one enzyme chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthetase, phosphatase, N-acetylneuraminate synthase and N-acylneuraminate cytidylyltransferase,
   preferably, wherein said cell is modified in the expression or activity of any one or more of said enzymes.
4. Cell according to any one of the previous embodiments, wherein said sialyltransferase is chosen from the list comprising alpha-2,3-sialyltransferase, alpha-2,6-sialyltransferase, and alpha-2,8-sialyltransferase.
5. Cell according to any one of the previous embodiments, wherein said sialic acid transporter is selected from the group of porters and P—P-bond-hydrolysis-driven transporters, wherein
   a) when said sialic acid transporter is selected from the group of porters, said sialic acid transporter is selected from
      the group of TCDB classes 2.A.1.12, 2.A.21.3 and 2.A.56.1, or
      the group of eggnog families 1MU0F, 1MUNB, 1MVHH, 1MWKH, 1R4VK, 1RJ8K, 1RMH7, 1RNAM, 1RPTE, 1RQFS, 1RZ3Z, 1SA02, 1TPNU, 1TPVE, 1TQCK, 1XTIW, 1XU43, 1Y733, 1Y7P0, 1YAT3, 258Y6, 25E9U, 25SBT, 25Y42, 26G88, 27G1B, 2IX6M, 2TQBC, 2TQW5, 36UQA, 3788V, 3F50Z, 3WG4Q, 3WP13, 3WTAI, 3WVF7, 3X1B1, 3XP7A, 3ZJ4T, 404JV, 41EHH, 4BDTU, 4C594, 4GX8Z, 4GY09, 4H21M, 4H9KW, 4H9WK, 4HB5K, 4IEK0, COG0477, COG0591, COG1593, COG2704, COG2814, COG3055, COG3090 and COG4666, or
      the PFAM list PF00083, PF00474, PF04290, PF06808, PF07690 and PF11874, or
      the interpro list IPR001734, IPR004681, IPR004742, IPR005828, IPR005829, IPR007387, IPR010656, IPR011701, IPR011851, IPR011853, IPR015915, IPR018212, IPR020846, IPR021814, IPR025966, IPR036259 and IPR038377, or
      NanT from Escherichia coli with SEQ ID NO 01, nanT from Yersinia enterocolitica subsp. palearctica serotype O:3 with SEQ ID NO 02, nanT from Buttiauxella brennerae ATCC 51605 with SEQ ID NO 03, nanT from E. coli KTE75 with SEQ ID NO 04, or a transporter from Mycoplasma alligatoris A21JP2 with SEQ ID NO 05, from M. capricolum subsp. capricolum with SEQ ID NO 06, from Staphylococcus aureus with SEQ ID NO 07, from Gemella sp. with SEQ ID NO 08, from Clostridioides difficile ATCC 9689 with SEQ ID NO 09, from Peptostreptococcus russellii with SEQ ID NO 10, from Bacillus obstructivus with SEQ ID NO 11, from S. aureus (strain MW2) with SEQ ID NO 12, from Staphylococcus sp. HMSC070A03 with SEQ ID NO 13, from Cetobacterium ceti with SEQ ID NO 14, from S. argensis with SEQ ID NO 15, from S. schleiferi with SEQ ID NO 16, from Alloiococcus otitis ATCC 51267 with SEQ ID NO 17, from Gracilibacillus dipsosauri with SEQ ID NO 18, from Lactobacillus versmoldensis with SEQ ID NO 19, from Lactobacillus sp. LL6 with SEQ ID NO 20, from Agrilactobacillus composti with SEQ ID NO 21, from L. crispatus with SEQ ID NO 22, from S. pasteuri with SEQ ID NO 23, from L. salivarius cp400 with SEQ ID NO 24, from L. apodemi with SEQ ID NO 25, from Selenomonas sp. oral taxon 478 with SEQ ID NO 26, from Paeniclostridium sordellii with SEQ ID NO 27, from Butyribacterium methylotrophicum with SEQ ID NO 28, from Alkalihalobacillus pseudofirmus with SEQ ID NO 29, from Streptococcus mitis (strain B6) with SEQ ID NO 30, from Aerococcus viridans with SEQ ID NO 31, from Anaerobiospirillum thomasii with SEQ ID NO 32, from Clostridium haemolyticum NCTC 8350 with SEQ ID NO 33, from C. thermobutyricum with SEQ ID NO 34 or SEQ ID NO 35, from Romboutsia ilealis with SEQ ID NO 36, from Megamonas funiformis YIT 11815 with SEQ ID NO 37, from Lysinibacillus sphaericus with SEQ ID NO 38, from uncultured Clostridium sp. with SEQ ID NO 39, from C. hiranonis with SEQ ID NO 40, from C. niameyense with SEQ ID NO 41, from Candidatus Arthromitus sp. SFB-turkey with SEQ ID NO 42, siaT_5 from Suttonella ornithocola with SEQ ID NO 43, a transporter from Pasteurella multocida subsp. multocida OH4807 with SEQ ID NO 44, from P. skyensis with SEQ ID NO 45, from [Haemophilus] felis with SEQ ID NO 46, from Gammaproteobacteria bacterium with SEQ ID NO 47, from [Pasteurella] mairii with SEQ ID NO 48, from Suttonella indologenes with SEQ ID NO 49, from Fusobacterium canifelinum with SEQ ID NO 50, from F. varium with SEQ ID NO 51, from F. necrophorum with SEQ ID NO 52, from Cardiobacterium hominis with SEQ ID NO 53, from Aggregatibacter actinomycetemcomitans RhAA1 with SEQ ID NO 54, or from P. bettyae CCUG 2042 with SEQ ID NO 55, or functional homolog, variant or derivative of any one of the SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 having at least 80% overall sequence identity to the full-length sequence of any one of said polypeptides with SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 respectively and having sialic acid transporter activity;
   b) when said sialic acid transporter is selected from the group of P—P-bond-hydrolysis-driven transporters, said sialic acid transporter is selected from
      the TCDB class 3.A.1.5, or
      the group of eggnog families 1MU8Z, 1MUZH, 1NU4K, 1R4 KB, 1RNFB, 1RS1R, 1SKPD, 1SMBI, 1Y72G, 1Y72H, 1Y7EZ, 1Y7FQ, COG0444, COG0601, COG0747, COG1173 and COG4608, or,
      the PFAM list PF00005, PF00496, PF00528 and PF08352, or
      the interpro list IPR000515, IPR000914, IPR003439, IPR003593, IPR006311, IPR013563, IPR017871, IPR027417, IPR030678, IPR035906 and IPR039424, or
      ABC transporter from Haemophilus paracuniculus with SEQ ID NO 56, 57, 58 or 59, or functional homolog, variant or derivative of any one of the SEQ ID NOs 56, 57, 58 or 59 having at least 80% overall sequence identity to the full-length sequence of any one of the polypeptides with SEQ ID NOs 56, 57, 58 or 59, respectively and having sialic acid transporter activity;
   wherein the TCDB classes are as defined by TCDB.org as released on 17 Jun. 2019, the eggnog families are as defined by eggnogdb 5.0.0 as released on November 2018, the PFAM lists are as defined by Pfam 32.0 as released on September 2018, the interpro lists are as defined by InterPro 75.0 as released on 4 Jul. 2019.
6. Cell according to any one of the previous embodiments, wherein said sialic acid transporter is selected from the group of transporters consisting of:
   a) the porters NanT from Escherichia coli with SEQ ID NO 01, nanT from Yersinia enterocolitica subsp. palearctica serotype O:3 with SEQ ID NO 02, nanT from Buttiauxella brennerae ATCC 51605 with SEQ ID NO 03, nanT from E. coli KTE75 with SEQ ID NO 04, or a transporter from Mycoplasma alligatoris A21JP2 with SEQ ID NO 05, from M. capricolum subsp. capricolum with SEQ ID NO 06, from Staphylococcus aureus with SEQ ID NO 07, from Gemella sp. with SEQ ID NO 08, from Clostridioides difficile ATCC 9689 with SEQ ID NO 09, from Peptostreptococcus russellii with SEQ ID NO 10, from Bacillus obstructivus with SEQ ID NO 11, from S. aureus (strain MW2) with SEQ ID NO 12, from Staphylococcus sp. HMSC070A03 with SEQ ID NO 13, from Cetobacterium ceti with SEQ ID NO 14, from S. argensis with SEQ ID NO 15, from S. schleiferi with SEQ ID NO 16, from Alloiococcus otitis ATCC 51267 with SEQ ID NO 17, from Gracilibacillus dipsosauri with SEQ ID NO 18, from Lactobacillus versmoldensis with SEQ ID NO 19, from Lactobacillus sp. LL6 with SEQ ID NO 20, from Agrilactobacillus composti with SEQ ID NO 21, from L. crispatus with SEQ ID NO 22, from S. pasteuri with SEQ ID NO 23, from L. salivarius cp400 with SEQ ID NO 24, from L. apodemi with SEQ ID NO 25, from Selenomonas sp. oral taxon 478 with SEQ ID NO 26, from Paeniclostridium sordellii with SEQ ID NO 27, from Butyribacterium methylotrophicum with SEQ ID NO 28, from Alkalihalobacillus pseudofirmus with SEQ ID NO 29, from Streptococcus mitis (strain B6) with SEQ ID NO 30, from Aerococcus viridans with SEQ ID NO 31, from Anaerobiospirillum thomasii with SEQ ID NO 32, from Clostridium haemolyticum NCTC 8350 with SEQ ID NO 33, from C. thermobutyricum with SEQ ID NO 34 or SEQ ID NO 35, from Romboutsia ilealis with SEQ ID NO 36, from Megamonas funiformis YIT 11815 with SEQ ID NO 37, from Lysinibacillus sphaericus with SEQ ID NO 38, from uncultured Clostridium sp. with SEQ ID NO 39, from C. hiranonis with SEQ ID NO 40, from C. niameyense with SEQ ID NO 41, from Candidatus Arthromitus sp. SFB-turkey with SEQ ID NO 42, siaT_5 from Suttonella ornithocola with SEQ ID NO 43, a transporter from Pasteurella multocida subsp. multocida OH4807 with SEQ ID NO 44, from P. skyensis with SEQ ID NO 45, from [Haemophilus] felis with SEQ ID NO 46, from Gammaproteobacteria bacterium with SEQ ID NO 47, from [Pasteurella] mairii with SEQ ID NO 48, from Suttonella indologenes with SEQ ID NO 49, from Fusobacterium canifelinum with SEQ ID NO 50, from F. varium with SEQ ID NO 51, from F. necrophorum with SEQ ID NO 52, from Cardiobacterium hominis with SEQ ID NO 53, from Aggregatibacter actinomycetemcomitans RhAA1 with SEQ ID NO 54, or from P. bettyae CCUG 2042 with SEQ ID NO 55 or functional homolog, variant or derivative of any one of the SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 having at least 80% overall sequence identity to the full-length sequence of any one of said polypeptides with SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 respectively and having sialic acid transporter activity;
      preferably, the porters NanT from Escherichia coli with SEQ ID NO 01, nanT from Yersinia enterocolitica subsp. palearctica serotype O:3 with SEQ ID NO 02, nanT from Buttiauxella brennerae ATCC 51605 with SEQ ID NO 03, nanT from E. coli KTE75 with SEQ ID NO 04, from L. salivarius cp400 with SEQ ID NO 24, or siaT_5 from Suttonella ornithocola with SEQ ID NO 43, or functional homolog, variant or derivative of any one of the SEQ ID NOs 01, 02, 03, 04, 24, or 43 having at least 80% overall sequence identity to the full-length sequence of any one of said preferred polypeptides with SEQ ID NOs 01, 02, 03, 04, 24, or 43, respectively and having sialic acid transporter activity; and
   b) the P—P-bond-hydrolysis-driven transporters from Haemophilus paracuniculus with SEQ ID NO 56, 57, 58 or 59, or functional homolog, variant or derivative of any one of the SEQ ID NOs 56, 57, 58 or 59 having at least 80% overall sequence identity to the full-length sequence of any one of the polypeptides with SEQ ID NOs 56, 57, 58 or 59, respectively and having sialic acid transporter activity.
7. Cell according to any one of the previous embodiments, wherein said sialic acid transporter is a transporter protein involved in the influx and/or efflux of sialic acid across the cytoplasmic membrane, preferably in and/or out of the cell.
8. Cell according to any one of the previous embodiments, wherein any one of said sialic acid transporter and/or enzyme involved in said pathway for production of said sialylated di- and/or oligosaccharide is presented to the cell in one or more gene expression modules wherein expression is regulated by one or more regulatory sequences.
9. Cell according to embodiment 8, wherein said expression modules are integrated in the host cell's genome and/or presented to the cell on a vector comprising plasmid, cosmid, phage, liposome or virus, which is to be stably transformed into said host cell.
10. Cell according to any one of the previous embodiments, wherein said cell is capable to synthesize N-acetylmannosamine (ManNAc), N-acetylmannosamine-6-phosphate (ManNAc-6-phosphate) and/or phosphoenolpyruvate (PEP).
11. Cell according to any one of the previous embodiments, wherein said cell is modified for enhanced synthesis and/or supply of PEP.
12. Cell according to any one of the previous embodiments, wherein said cell is further capable to synthesize any one or more nucleotide-activated sugars.
13. Cell according to embodiment 12, wherein said nucleotide-activated sugar is chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-fucose (GDP-Fuc), GDP-rhamnose and UDP-xylose.
14. Cell according to any one of embodiment 12 or 13, wherein said nucleotide-activated sugar is synthesized by at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, glucosamine 6-phosphate N-acetyltransferase, N-acetylglucosamine-6-phosphate phosphatase, N-acetyl mannosamine-6-phosphate phosphatase, N-acetylmannosamine kinase, N-acetylmannosamine-6-phosphate 2-epimerase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphatase, CMP-sialic acid synthetase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-GalNAc pyrophosphorylase.
15. Cell according to any one of the previous embodiments, wherein said cell further expresses a glycosyltransferase chosen from the list comprising fucosyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
   preferably, wherein said cell is modified in the expression or activity of at least one of said glycosyltransferases,
   preferably, said fucosyltransferase is chosen from the list comprising alpha-1,2-fucosyltransferase, alpha-1,3-fucosyltransferase, alpha-1,4-fucosyltransferase, alpha1-,3/4-fucosyltransferase and alpha-1,6-fucosyltransferase,
   preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
   preferably, said glucosyltransferase is chosen from the list comprising alpha-glucosyltransferase, beta-1,2-glucosyltransferase, beta-1,3-glucosyltransferase and beta-1,4-glucosyltransferase,
   preferably, said mannosyltransferase is chosen from the list comprising alpha-1,2-mannosyltransferase, alpha-1,3-mannosyltransferase and alpha-1,6-mannosyltransferase,
   preferably, said N-acetylglucosaminyltransferase is chosen from the list comprising galactoside beta-1,3-N-acetylglucosaminyltransferase and beta-1,6-N-acetylglucosaminyltransferase,
   preferably, said N-acetylgalactosaminyltransferase is chosen from the list comprising alpha-1,3-N-acetylgalactosaminyltransferase.
16. Cell according to any one of the previous embodiments, wherein said cell comprises a fucosylation pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-fucokinase/GDP-fucose pyrophosphorylase and fucosyltransferase.
17. Cell according to any one of the previous embodiments, wherein said cell comprises a galactosylation pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase and galactosyltransferase.
18. Cell according to any one of the previous embodiments, wherein said cell comprises an N-acetylglucosaminylation pathway comprising at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase.
19. Cell according to any one of the previous embodiments, wherein said cell comprises a modification for reduced production of acetate.
20. Cell according to any one of the previous embodiments, wherein said cell further comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IcIR, Ion protease, glucose-specific translocating phosphotransferase enzyme IIBC component ptsG, glucose-specific translocating phosphotransferase (PTS) enzyme IIBC component maIX, enzyme IIA beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.
21. Cell according to any one of previous embodiments, wherein said cell comprises a catabolic pathway for selected mono-, di- or oligosaccharides which is at least partially inactivated, the mono-, di-, or oligosaccharides being involved in and/or required for the synthesis of said sialylated di- and/or oligosaccharide.
22. Cell according to any one of the previous embodiments, wherein said cell is using a precursor for the synthesis of said sialylated di- and/or oligosaccharide, said precursor being fed to the cell from the cultivation medium.
23. Cell according to any one of the previous embodiments, wherein said cell is producing a precursor for the synthesis of said sialylated di- and/or oligosaccharide.
24. Cell according to any one of the previous embodiments, wherein said cell produces 90 g/L or more of said sialylated di- and/or oligosaccharide in the whole broth and/or supernatant and/or wherein said sialylated di- and/or oligosaccharide in the whole broth and/or supernatant has a purity of at least 80% measured on the total amount of sialylated di- and/or oligosaccharide and its precursor produced by said cell in the whole broth and/or supernatant, respectively.
25. Cell according to any one of the previous embodiments, wherein said sialylated di- and/or oligosaccharide is chosen from the list comprising a milk oligosaccharide, O-antigen, an oligosaccharide repeat present in capsular polysaccharides, peptidoglycan, amino-sugars and Lewis-type antigen oligosaccharides, preferably said milk oligosaccharide is a mammalian milk oligosaccharide, more preferably said milk oligosaccharide is a human milk oligosaccharide.
26. Cell according to any one of the previous embodiments, wherein said cell is a bacterium, fungus or yeast,
   preferably said bacterium is of an Escherichia coli strain, more preferably of an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces.
27. Cell according to any one of the previous embodiments, wherein said cell is stably cultured in a medium.
28. Cell according to any one of the previous embodiments, wherein the cell is capable to synthesize a mixture of oligosaccharides comprising at least one sialylated oligosaccharide.
29. Cell according to any one of the previous embodiments, wherein the cell is capable to synthesize a mixture of di- and oligosaccharides comprising at least one sialylated di- and/or oligosaccharide.
30. Method to produce a sialylated di- and/or oligosaccharide by a cell of a microorganism, the method comprising the steps of:
   1) providing a cell according to any one of embodiments 1 to 29, and
   2) cultivating said cell under conditions permissive to produce said sialylated di- and/or oligosaccharide,
   3) preferably, separating said sialylated di- and/or oligosaccharide from said cultivation.
31. Method according to embodiment 30, wherein >95% of said sialic acid synthesized by said cell is used for the production of said sialylated di- and/or oligosaccharide.
32. Method according to any one of embodiment 30 or 31, the method further comprising at least one of the following steps:
   i) Use of a culture medium comprising at least one precursor and/or acceptor for the production of said sialylated di- and/or oligosaccharide, and/or
   ii) Adding to the culture medium at least one precursor and/or acceptor feed for the production of said sialylated di- and/or oligosaccharide.
33. Method according to any one of embodiments 30 to 32, the method further comprising at least one of the following steps:
   i) Use of a culture medium comprising at least one precursor and/or acceptor;
   ii) Adding to the culture medium in a reactor at least one precursor and/or acceptor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed;
   iii) Adding to the culture medium in a reactor at least one precursor and/or acceptor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed and wherein preferably, the pH of said precursor and/or acceptor feed is set between 2.0 and 10.0 and wherein preferably, the temperature of said precursor and/or acceptor feed is kept between 20° C. and 80° C.;
   iv) Adding at least one precursor and/or acceptor feed in a continuous manner to the culture medium over the course of 1 day, 2 days, 3 days, 4 days, 5 days by means of a feeding solution;
   v) Adding at least one precursor and/or acceptor feed in a continuous manner to the culture medium over the course of 1 day, 2 days, 3 days, 4 days, 5 days by means of a feeding solution and wherein preferably, the pH of said feeding solution, preferably said precursor and/or acceptor feed, is set between 2.0 and 10.0, preferably between 3 and 7, and wherein preferably, the temperature of said feeding solution, preferably said precursor and/or acceptor feed, is kept between 20° C. and 80° C.;
   said method resulting in a sialylated di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
34. Method according to any one of claims 30 to 32, the method further comprising at least one of the following steps:
   i) Use of a culture medium comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter);
   ii) Use of a culture medium comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter);
   iii) Adding to the culture medium in a reactor a precursor feed comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter), preferably in a continuous manner, and preferably so that the final volume of the culture medium is not more than three fold, preferably not more than two-fold, more preferably less than 2-fold of the volume of the culture medium before the addition of said precursor feed;
   iv) Adding to the culture medium in a reactor an acceptor feed comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said acceptor feed;
   v) Adding to the culture medium in a reactor a precursor feed comprising at least 50, more preferably at least 75, more preferably at least 100, more preferably at least 120, more preferably at least 150 grams of precursor per litre of initial reactor volume wherein the total reactor volume ranges from 250 mL to 10.000 m³ (cubic meter), preferably in a continuous manner, and preferably so that the final volume of the culture medium is not more than three fold, preferably not more than two-fold, more preferably less than 2-fold of the volume of the culture medium before the addition of said precursor feed and wherein preferably, the pH of said precursor feed is set between 2.0 and 10.0 and wherein preferably, the temperature of said precursor feed is kept between 20° C. and 80° C.;
   vi) Adding to the culture medium in a reactor an acceptor feed comprising at least 50, more preferably at least 75, more preferably at least 100, more preferably at least 120, more preferably at least 150 grams of acceptor per litre of initial reactor volume wherein the total reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said acceptor feed and wherein preferably, the pH of said acceptor feed is set between 2.0 and 10.0 and wherein preferably, the temperature of said acceptor feed is kept between 20° C. and 80° C.;
   vii) Adding a precursor and/or acceptor feed in a continuous manner to the culture medium over the course of 1 day, 2 days, 3 days, 4 days, 5 days by means of a feeding solution;
   viii) Adding a precursor feed in a continuous manner to the culture medium over the course of 1 day, 2 days, 3 days, 4 days, 5 days by means of a precursor feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said precursor feeding solution is set between 2.0 and 10.0 and wherein preferably, the temperature of said precursor feeding solution is kept between 20° C. and 80° C.;
   ix) Adding an acceptor feed in a continuous manner to the culture medium over the course of 1 day, 2 days, 3 days, 4 days, 5 days by means of an acceptor feeding solution and wherein the concentration of said acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably, the pH of said acceptor feeding solution is set between 2.0 and 10.0 and wherein preferably, the temperature of said acceptor feeding solution is kept between 20° C. and 80° C.;
   said method resulting in a sialylated di- and/or oligosaccharide with a concentration of at least 50 g/L,
   preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
35. Method according to any one of embodiments 30 to 32, the method further comprising at least one of the following steps:
   i) Use of a culture medium comprising at least 50, more preferably at least 75, more preferably at least 100, more preferably at least 120, more preferably at least 150 grams of lactose per litre of initial reactor volume wherein the total reactor volume ranges from 250 mL to 10.000 m³ (cubic meter);
   ii) Adding to the culture medium in a reactor a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   iii) Adding to the culture medium in a reactor a lactose feed comprising at least 50, more preferably at least 75, more preferably at least 100, more preferably at least 120, more preferably at least 150 grams of lactose per litre of initial reactor volume wherein the total reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably, the pH of said lactose feed is set between 2.0 and 10.0, preferably between 3 and 7, and wherein preferably, the temperature of said lactose feed is kept between 20° C. and 80° C.;
   iv) Adding a lactose feed in a continuous manner to the culture medium over the course of 1 day, 2 days, 3 days, 4 days, 5 days by means of a feeding solution;
   v) Adding a lactose feed in a continuous manner to the culture medium over the course of 1 day, 2 days, 3 days, 4 days, 5 days by means of a lactose feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said lactose feeding solution is set between 2.0 and 10.0, preferably between 3 and 7, and wherein preferably the temperature of said lactose feeding solution is kept between 20° C. and 80° C.;
   said method resulting in a sialylated oligosaccharide produced from said lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
36. Method according to embodiment 35, wherein the lactose feed is accomplished by adding lactose from the beginning of the cultivation in a concentration of at least 5 mM, preferably in a concentration of 30, 40, 50, 60, 70, 80, 90, 100, 150 mM, more preferably in a concentration >300 mM.
37. Method according to any one of embodiment 35 or 36, wherein said lactose feed is accomplished by adding lactose to the cultivation medium in a concentration, such, that throughout the production phase of the cultivation a lactose concentration of at least 5 mM, preferably 10 mM or 30 mM is obtained.
38. Method according to any one of embodiments 30 to 37, wherein the host cells are cultivated for at least about 60, 80, 100, or about 120 hours or in a continuous manner.
39. Method according to any one of embodiments 30 to 38, wherein said cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, wherein said carbon source is chosen from the list comprising glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.
40. Method according to any one of embodiments 30 to 39, wherein said cell uses at least one precursor for the synthesis of said sialylated di- and/or oligosaccharide, preferably said cell uses two or more precursors for the synthesis of said sialylated di- and/or oligosaccharide.
41. Method according to any one of embodiments 30 to 40, wherein the culture medium contains at least one compound selected from the group comprising lactose, galactose, sialic acid, fucose, GlcNAc, GalNAc, lacto-N-biose (LNB), N-acetyllactosamine (LacNAc).
42. Method according to any one of embodiments 30 to 41, wherein a first phase of exponential cell growth is provided by adding a carbon-based substrate, preferably glucose or sucrose, to the culture medium before the precursor, preferably lactose, is added to the culture medium in a second phase.
43. Method according to any one of embodiments 30 to 42, wherein said cell is producing at least one precursor for the synthesis of said sialylated di- and/or oligosaccharide.
44. Method according to any one of embodiments 30 to 43, wherein said precursor for the synthesis of said sialylated di- and/or oligosaccharide is completely converted into said sialylated di- and/or oligosaccharide.
45. Method according to any one of embodiments 30 to 44, wherein the sialylated di- and/or oligosaccharide is separated from the culture medium and/or the cell.
46. Method according to any one of embodiments 30 to 45, wherein said separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis, affinity chromatography, ion exchange chromatography, hydrophobic interaction chromatography and/or gel filtration, ligand exchange chromatography, electrodialysis.
47. Method according to any one of embodiments 30 to 46, wherein said method further comprises purification of said sialylated di- and/or oligosaccharide.
48. Method according to embodiment 47, wherein said purification comprises at least one of the following steps: use of activated charcoal or carbon, use of charcoal, nanofiltration, ultrafiltration, electrophoresis, enzymatic treatment or ion exchange, temperature adjustment, pH adjustment, pH adjustment with an alkaline or acidic solution, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.
49. Use of a sialic acid transporter selected from SEQ ID NOs 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59, or functional homolog, variant or derivative of any one of the SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59 having at least 80% overall sequence identity to the full-length sequence of any one of said polypeptides with SEQ ID NOs 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59, respectively, and having sialic acid transporter activity in the fermentative production of a sialylated di- and/or oligosaccharide.
50. Use of a cell according to any one of embodiments 1 to 27 for production of a sialylated di- and/or oligosaccharide.
51. Use of a cell according to embodiment 28 for production of a mixture of oligosaccharides comprising at least one sialylated oligosaccharide.
52. Use of a cell according to embodiment 29 for production of a mixture of di- and oligosaccharides comprising at least one sialylated di- and/or oligosaccharide.
53. Use of a method according to any one of embodiments 30 to 48 for production of a sialylated di- and/or oligosaccharide.

The invention will be described in more detail in the examples. The following examples will serve as further illustration and clarification of the present invention and are not intended to be limiting.

### EXAMPLES

### Example 1. Identification of Protein Domains

An HMM is a probabilistic model called profile hidden Markov models. It characterizes a set of aligned proteins into a position-specific scoring system. Amino acids are given a score at each position in the sequence alignment according to the frequency by which they occur (Eddy, S. R. 1998. Profile hidden Markov models. Bioinformatics. 14: 755-63). HMMs have wide utility, as is clear from the numerous databases that use this method for protein classification, including Pfam, InterPro, SMART, TIGRFAM, PIRSF, PANTHER, SFLD, Superfamily and Gene3D.

HMMsearch from the HMMER package 3.2.1 (http://hmmer.org/) as released on 13 Jun. 2019 can use this HMM to search sequence databases for sequence homologs. Sequence databases that can be used are for example, but not limited to the NCBI nr Protein Database (NR; https://www.ncbi.nlm.nih.gov/protein), UniProt Knowledgebase (UniProtKB, https://www.uniprot.org/help/uniprotkb) and the SWISS-PROT database (https://web.expasy.org/docs/swiss-prot_guideline.html).

Sialic acid transporters were classified based on InterPro 75.0 (https://www.ebi.ac.uk/interpro/) as released on 4 Jul. 2019 and PFAM domains using Pfam 32.0 (https://pfam.xfam.org/) as released on September 2018. The Pfam and InterPro databases are a large collection of protein families. Other protein domains like SMART (http://smart.embl-heidelberg.de/), TIGRFAM (https://www.jcvi.org/tigrfams), PIRSF (https://proteininformationresource.org/pirwww/dbinfo/pirsf.shtml), PANTHER (http://pantherdb.org/), SFLD (http://sfld.rbvi.ucsf.edu/archive/django/index.html), Superfamily (http://supfam.org/) and Gene3D (http://gene3d.biochem.ucl.ac.uk/Gene3D/), NCBI Conserved Domains (https://www.ncbi.nlm.nih.gov/Structure/cdd/wrpsb.cgi) can also be used.

Identification of the PFAM domains was done by an online search on https://pfam.xfam.org/search#tabview=tab1 as released on September 2018. The HMM for the obtained family was downloaded in ‘Curation & model’. HMMsearches with this model to the protein databases will identify new family members. Sequences comprising the InterPro hit can also be downloaded from the PFAM website.

Identification of the InterPro (super)families, domains and sites was done by using the online tools on https://www.ebi.ac.uk/interpro/or a standalone version of InterProScan (https://www.ebi.ac.uk/interpro/download.html), both based on InterPro 75.0 as released on 4 Jul. 2019. InterPro is a composite database combining the information of many databases of protein motifs and domains. The HMM of the InterPro domain and/or (super)families can be obtained from InterProScan and can be used to identify new family members in the protein databases. Sequences comprising the InterPro hit can also be downloaded from the InterPro website (‘Protein Matched’) or can be queried on the UniProt website (https://www.uniprot.org).

### Example 2. Calculation of Percentage Identity Between Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48: 443-453) to find the global (i.e. spanning the full-length sequences) alignment of two sequences that maximizes the number of matches and minimizes the number of gaps. The BLAST algorithm (Altschul et al., J. Mol. Biol. (1990) 215: 403-10) calculates the global percentage sequence identity (i.e. over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity ((i.e. spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147(1); 195-7). For the purposes of this invention, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

### Example 3. Identification of Sialic Acid Transporters

Genes in the neighborhood of IPR005264 (N-acetylneuraminate lyase) and IPR023945 (N-acetylmannosamine kinase) containing genes were extracted from Uniprot ((https://www.uniprot.org) on 4 Mar. 2021. The identifiers were imported into the EFI-GENOME NEIGHBORHOOD TOOL (https://efi.igb.illinois.edu//efi-gnt/index.php) using default parameters. Amino acid sequences were filtered on completeness (from start codon to stop codon) and clustered using CD-HIT (http://weizhongli-lab.org/cd-hit/) with a sequence identity threshold of 80%. The representative sequences are listed below. The sialic acid transporters belong to four different TCDB classes. The first group are MFS transporters of TCDB class 2.A.1.12: nanT from E. coli K12 MG1655 with SEQ ID NO 01, A0A0H3NU41 (nanT) from Yersinia enterocolitica subsp. palearctica serotype O:3 with SEQ ID NO 02, A0A1B71K14 (nanT) from Buttiauxella brennerae ATCC 51605 with SEQ ID NO 03 and L3PL77 (nanT) from E. coli KTE75 with SEQ ID NO 04. The second group belongs to TCDB class 2.A.21.3: D4XVE0 from Mycoplasma alligatoris A21JP2 with SEQ ID NO 05, A0A0C2VH96 from M. capricolum subsp. capricolum with SEQ ID NO 06, A0A0D3Q460 from Staphylococcus aureus with SEQ ID NO 07, A0A533HZC4 from Gemella sp. with SEQ ID NO 08, A0A125V716 from Clostridioides difficile ATCC 9689 with SEQ ID NO 09, A0A1H8K9Q3 from Peptostreptococcus russellii with SEQ ID NO 10, A0A1L8ZSQ8 from Bacillus obstructivus with SEQ ID NO 11, A0A0H3JVD2 from S. aureus (strain MW2) with SEQ ID NO 12, A0A1S1F7G7 from Staphylococcus sp. HMSC070A03 with SEQ ID NO 13, A0A1T4NAC5 from Cetobacterium ceti with SEQ ID NO 14, A0A2K4FCH9 from S. argensis with SEQ ID NO 15, A0A0M4UEF4 from S. schleiferi with SEQ ID NO 16, K9EWT1 from Alloiococcus otitis ATCC 51267 with SEQ ID NO 17, A0A317KV18 from Gracilibacillus dipsosauri with SEQ ID NO 18, A0A0R1S9A9 from Lactobacillus versmoldensis with SEQ ID NO 19, A0A556U9X1 from Lactobacillus sp. LL6 with SEQ ID NO 20, A0A0R1Y239 from Agrilactobacillus composti with SEQ ID NO 21, A0A2N5L0P0 from L. crispatus with SEQ ID NO 22, A0A431ZS05 from S. pasteuri with SEQ ID NO 23, LSCP400_11881 from L. salivarius cp400 with SEQ ID NO 24, A0A0R1TYU7 from L. apodemi with SEQ ID NO 25, A0A0K1FGD7 from Selenomonas sp. oral taxon 478 with SEQ ID NO 26, A0A0C7PUW9 from Paeniclostridium sordellii with SEQ ID NO 27, A0A1E7RYL9 from Butyribacterium methylotrophicum with SEQ ID NO 28, A0A1Q8TWT1 from Alkalihalobacillus pseudofirmus with SEQ ID NO 29, D3H850 from Streptococcus mitis (strain B6) with SEQ ID NO 30, A0A2X0UKU9 from Aerococcus viridans with SEQ ID NO 31, A0A2X0VQ52 from Anaerobiospirillum thomasii with SEQ ID NO 32, A0A0A01153 from Clostridium haemolyticum NCTC 8350 with SEQ ID NO 33, A0A1V4SXX4 or N9WAN6 from C. thermobutyricum with SEQ ID NO 34 or SEQ ID NO 35 respectively, A0A1V1HZG9 from Romboutsia ilealis with SEQ ID NO 36, H3KA61 from Megamonas funiformis YIT 11815 with SEQ ID NO 37, A0A3P6L5Y9 from Lysinibacillus sphaericus with SEQ ID NO 38, A0A1C5UWV6 from uncultured Clostridium sp. with SEQ ID NO 39, B6FWE2 from C. hiranonis with SEQ ID NO 40, A0A6M0RCY3 from C. niameyense with SEQ ID NO 41 and A0A1B7LK65 from Candidatus Arthromitus sp. SFB-turkey with SEQ ID NO 42. The third group belongs to TCDB class 2.A.56.1: A0A380MYE0 (siaT_5) from Suttonella ornithocola with SEQ ID NO 43, A0A0E3V465 from Pasteurella multocida subsp. multocida OH4807 with SEQ ID NO 44, A0A1H7ZQG5 from P. skyensis with SEQ ID NO 45, A0A1T0B8S6 from [Haemophilus] felis with SEQ ID NO 46, A0A2G6EKL2 from Gammaproteobacteria bacterium with SEQ ID NO 47, A0A379B306 from [Pasteurella] mairii with SEQ ID NO 48, A0A380MWW9 from Suttonella indologenes with SEQ ID NO 49, A0A3P1USS0 from Fusobacterium canifelinum with SEQ ID NO 50, A0A448MCK2 from F. varium with SEQ ID NO 51, A0A4V1QY13 from F. necrophorum with SEQ ID NO 52, C8N6B9 from Cardiobacterium hominis with SEQ ID NO 53, H0KGY6 from Aggregatibacter actinomycetemcomitans RhAA1 with SEQ ID NO 54 and 13DJD6 from P. bettyae CCUG 2042 with SEQ ID NO 55. A fourth group belongs to the ABC transporter class 3.A.1.5 and are found in Haemophilus paracuniculus: B0187_07585 with SEQ ID NO 56, B0187_07590 with SEQ ID NO 57, B0187_07595 with SEQ ID NO 58 and B0187_07600 with SEQ ID NO 59.

### Example 4. Materials and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR, Leuven, Belgium). The minimal medium used in the cultivation experiments in 96-well plates or in shake flasks contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4·7H2O, 30 g/L sucrose or 30 g/L glycerol, 1 ml/L vitamin solution, 100 μl/L molybdate solution, and 1 mL/L selenium solution. As specified in the respective examples, 20 g/L lactose, 20 g/L LacNAc and/or 20 g/L LNB were additionally added to the medium as precursor(s). The minimal medium was set to a pH of 7 with 1M KOH. Vitamin solution consisted of 3.6 g/L FeCl2·4H2O, 5 g/L CaCl2·2H2O, 1.3 g/L MnCl2·2H2O, 0.38 g/L CuCl2·2H2O, 0.5 g/L CoCl2·6H2O, 0.94 g/L ZnCl2, 0.0311 g/L H3BO4, 0.4 g/L Na2EDTA·2H2O and 1.01 g/L thiamine·HCl. The molybdate solution contained 0.967 g/L NaMoO4·2H2O. The selenium solution contained 42 g/L Seo2. The minimal medium for fermentations contained 6.75 g/L NH4Cl, 1.25 g/L (NH4)2SO4, 2.93 g/L KH2PO4 and 7.31 g/L KH2PO4, 0.5 g/L NaCl, 0.5 g/L MgSO4·7H2O, 30 g/L sucrose or 30 g/L glycerol, 1 mL/L vitamin solution, 100 μL/L molybdate solution, and 1 mL/L selenium solution with the same composition as described above. As specified in the respective examples, 20 g/L lactose, 20 g/L LacNAc and/or 20 g/L LNB were additionally added to the medium as precursor(s). Complex medium was sterilized by autoclaving (121° C., 21 min) and minimal medium by filtration (0.22 μm Sartorius). When necessary, the medium was made selective by adding an antibiotic: e.g. chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F, phi80dlacZLΔM15, Δ(lacZYA-argF) U169, deoR, recA1, endA1, hsdR17(rk⁻, mk⁺), phoA, supE44, lambda⁻, thi-1, gyrA96, relA1) bought from Invitrogen.

### Strains and Mutations

Escherichia coli K12 MG1655 [Δ⁻, F, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an OD₆₀₀ nm of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with DpnI, re-purified from an agarose gel, and suspended in elution buffer (5 mM Tris, pH 8.0). Selected mutants were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers.

In one example for sialic acid production, the mutant strain was derived from E. coli K12 MG1655 comprising knock-outs of the E. coli nanA and nanT genes and genomic knock-ins of constitutive transcriptional units containing one or more sialic acid transporters chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, a glucosamine 6-phosphate N-acetyltransferase like e.g. GNA1 from Saccharomyces cerevisiae (UniProt ID P43577), an N-acylglucosamine 2-epimerase like e.g. AGE from Bacteroides ovatus (UniProt ID A7LVG6) and an N-acetylneuraminate synthase like e.g. NeuB from Neisseria meningitidis (UniProt ID E0NCD4) or from Campylobacter jejuni (UniProt ID Q93MP9).

Alternatively and/or additionally, sialic acid production can be obtained by knock-outs of the E. coli nanA and nanTgenes and genomic knock-ins of constitutive transcriptional units containing one or more sialic acid transporters chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, a UDP-N-acetylglucosamine 2-epimerase like e.g. NeuC from Campylobacter jejuni (UniProt ID Q93MP8) and an N-acetylneuraminate synthase like e.g. NeuB from N. meningitidis (UniProt ID E0NCD4) or from C. jejuni (UniProt ID Q93MP9). Alternatively and/or additionally, sialic acid production can be obtained by knock-outs of the E. coli nanA and nanTgenes and genomic knock-ins of constitutive transcriptional units containing one or more sialic acid transporters chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, an phosphoglucosamine mutase like e.g. glmM from E. coli (UniProt ID P31120), a bifunctional N-acetylglucosamine-1-phosphate uridyltransferase/glucosamine-1-phosphate acetyltransferase like e.g. glmU from E. coli (UniProt ID P0ACC7), a UDP-N-acetylglucosamine 2-epimerase like e.g. NeuC from C. jejuni (UniProt ID Q93MP8) and an N-acetylneuraminate synthase like e.g. NeuB from N. meningitidis (UniProt ID E0NCD4) or from C. jejuni (UniProt ID Q93MP9).

Alternatively and/or additionally, sialic acid production can be obtained by knock-outs of the E. coli nanA and nanTgenes and genomic knock-ins of constitutive transcriptional units containing one or more sialic acid transporters chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, a bifunctional UDP-GlcNAc 2-epimerase/N-acetylmannosamine kinase like e.g. from Mus musculus (strain C57BL/6J) (UniProt ID Q91WG8), an N-acylneuraminate-9-phosphate synthetase like e.g. from Syntrophorhabdus sp. PtaU1.Bin058 with SEQ ID NO 63 and an N-acylneuraminate-9-phosphatase like e.g. from Candidatus Magnetomorum sp. HK-1 (UniProt ID KPA15328.1) or from Bacteroides thetaiotaomicron (strain ATCC 29148) (UniProt ID Q8A712).

Alternatively and/or additionally, sialic acid production can be obtained by knock-outs of the E. coli nanA and nanTgenes and genomic knock-ins of constitutive transcriptional units containing one or more sialic acid transporters chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, a phosphoglucosamine mutase like e.g. glmM from E. coli (UniProt ID P31120), a bifunctional N-acetylglucosamine-1-phosphate uridyltransferase/glucosamine-1-phosphate acetyltransferase like e.g. glmU from E. coli (UniProt ID P0ACC7), a bifunctional UDP-GlcNAc 2-epimerase/N-acetylmannosamine kinase like e.g. from M. musculus (strain C57BL/6J) (UniProt ID Q91WG8), an N-acylneuraminate-9-phosphate synthetase like e.g. from Syntrophorhabdus sp. PtaU1.Bin058 with SEQ ID NO 63 and an N-acylneuraminate-9-phosphatase like e.g. from Candidatus Magnetomorum sp. HK-1 (UniProt ID KPA15328.1) or from Bacteroides thetaiotaomicron (strain ATCC 29148) (UniProt ID Q8A712).

Sialic acid production can further be optimized in the mutant E. coli strain with genomic knock-outs of any one or more of the E. coli genes comprising nagA, nagB, nagC, nagD, nagE, nanE, nanK, manX, manYand manZ as described in WO18122225, and/or genomic knock-outs of the E. coli genes comprising any one or more of poxB, IdhA, adhE, aldB, pflA, pflC, ybiY, ackA and/or pta and with genomic knock-ins of constitutive transcriptional units comprising any one or more of an L-glutamine-D-fructose-6-phosphate aminotransferase like e.g. the mutant glmS*54 from E. coli (differing from the wild-type E. coli glmS with UniProt ID P17169 by an A39T, an R250C and an G472S mutation) and an acetyl-CoA synthetase like e.g. acs from E. coli (UniProt ID P27550).

For sialylated oligosaccharide production, said sialic acid production strains further need to express one or more copies of an N-acylneuraminate cytidylyltransferases like e.g. NeuA from Pasteurella multocida with SEQ ID NO 61, NeuA from C. jejuni (UniProt ID Q93MP7) or NeuA from Haemophilus influenzae with SEQ ID NO 62, and one or more copies of a beta-galactoside alpha-2,3-sialyltransferase like e.g. SEQ ID NO 64 from P. multocida subsp. multocida str. Pm70 and SEQ ID NO 65 (NmeniST3) from N. meningitidis, a beta-galactoside alpha-2,6-sialyltransferase like e.g. (PdST6) from Photobacterium damselae (UniProt ID 066375) or a PdST6-like polypeptide consisting of amino acid residues 108 to 497 of UniProt ID 066375 having beta-galactoside alpha-2,6-sialyltransferase activity and P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity, and/or an alpha-2,8-sialyltransferase like e.g. from M. musculus (UniProt ID Q64689). Constitutive transcriptional units of any one or more of the N-acylneuraminate cytidylyltransferases and the sialyltransferases can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like e.g. the E. coli LacY (UniProt ID P02920). All mutant strains producing sialic acid, CMP-sialic acid and/or sialylated oligosaccharides could optionally be adapted for growth on sucrose via genomic knock-ins of constitutive transcriptional units containing a sucrose transporter like e.g. CscB from E. coli W (UniProt ID E0IXR1), a fructose kinase like e.g. Frk originating from Z. mobilis (UniProt ID Q03417) and a sucrose phosphorylase like e.g. from B. adolescentis (UniProt ID A0ZZH6).

For GFP-fucose production in the E. coli strains producing sialic acid, the mutant strains in these examples were further modified comprising knock-outs of the E. coli wcaJ and thyA genes and genomic knock-ins of constitutive transcriptional units containing a sucrose transporter like e.g. CscB from E. coli W (UniProt ID E0IXR1), a fructose kinase like e.g. Frk originating from Z. mobilis (UniProt ID Q03417) and a sucrose phosphorylase (SP) like e.g. from B. adolescentis (UniProt ID A0ZZH6). For production of fucosylated oligosaccharides, the mutant GDP-fucose production strain was additionally modified with expression plasmids comprising constitutive transcriptional units for an alpha-1,2-fucosyltransferase like e.g. HpFutC from H. pylori with SEQ ID NO 60 and/or an alpha-1,3-fucosyltransferase like e.g. HpFucT from H. pylori (UniProt ID 030511) and with a constitutive transcriptional unit for the E. coli thyA (UniProt ID P0A884) as selective marker. The constitutive transcriptional units of the fucosyltransferase genes could also be present in the mutant E. coli strain via genomic knock-ins. GDP-fucose production can further be optimized in the mutant E. coli strain by genomic knock-outs of any one or more of the E. coli genes comprising glgC, agp, pfkA, pfkB, pgi, arcA, iclR, pgi and Ion as described in WO2016075243 and WO2012007481. GDP-fucose production can additionally be optimized comprising genomic knock-ins of constitutive transcriptional units for a mannose-6-phosphate isomerase like e.g. manA from E. coli (UniProt ID P00946), a phosphomannomutase like e.g. manB from E. coli (UniProt ID P24175), a mannose-1-phosphate guanylyltransferase like e.g. manC from E. coli (UniProt ID P24174), a GDP-mannose 4,6-dehydratase like e.g. gmd from E. coli (UniProt ID P0AC88) and a GDP-L-fucose synthase like e.g. fcl from E. coli (UniProt ID P32055). GDP-fucose production can also be obtained by genomic knock-outs of the E. coli fucK and fucI genes and genomic knock-ins of constitutive transcriptional units containing a fucose permease like e.g. fucP from E. coli (UniProt ID P11551) and a bifunctional fucose kinase/fucose-1-phosphate guanylyltransferase like e.g. fkp from Bacteroides fragilis (UniProt ID SUV40286.1). If the mutant strains producing sialic acid and GDP-fucose were intended to make fucosylated lactose structures, the strains were additionally modified with genomic knock-outs of the E. coli LacZ, LacY and LacA genes and with a genomic knock-in of a constitutive transcriptional unit for a lactose permease like e.g. the E. coli LacY (UniProt ID P02920). Furthermore, if the mutant strains were also intended to make sialylated structures, the strains were additionally modified with genomic knock-ins or expression plasmids comprising constitutive transcriptional units for one or more copies of an N-acylneuraminate cytidylyltransferase like e.g. NeuA from P. multocida with SEQ ID NO 61, NeuA from C. jejuni (UniProt ID Q93MP7) or NeuA from H. influenzae with SEQ ID NO 62, and one or more copies of a beta-galactoside alpha-2,3-sialyltransferase like e.g. SEQ ID NO 64 from P. multocida subsp. multocida str. Pm70 and SEQ ID NO 65 (NmeniST3) from N. meningitidis, a beta-galactoside alpha-2,6-sialyltransferase like e.g. PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity and P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity and/or an alpha-2,8-sialyltransferase like e.g. from M. musculus (UniProt ID Q64689).

For production of LN3 (GlcNAc-b1,3-Gal-b1,4-Glc) in the E. coli strains producing sialic acid, the mutant strains in these examples were further modified comprising genomic knock-outs of the E. coli LacZ, LacY and LacA genes and genomic knock-ins of constitutive transcriptional units for a lactose permease like e.g. the E. coli LacY (UniProt ID P02920) and a galactoside beta-1,3-N-acetylglucosaminyltransferase like e.g. LgtA from N. meningitidis with SEQ ID NO 66.

For production of LN3-derived oligosaccharides like lacto-N-tetraose (LNT) and lacto-N-neotetraose (LNnT) the mutant LN3 producing strains were further modified with a constitutive transcriptional unit delivered to the strain either via genomic knock-in or from an expression plasmid for an N-acetylglucosamine beta-1,3-galactosyltransferase like e.g. WbgO from E. coli O55:H7 (UniProt ID D3QY14) to produce LNT or for an N-acetylglucosamine beta-1,4-galactosyltransferase like e.g. LgtB from N. meningitidis (UniProt ID Q51116) to produce LNnT. Optionally, multiple copies of the galactoside beta-1,3-N-acetylglucosaminyltransferase, the N-acetylglucosamine beta-1,3-galactosyltransferase and/or the N-acetylglucosamine beta-1,4-galactosyltransferase encoding genes could be added to the mutant E. coli strains. In addition, the strains can optionally be modified for enhanced UDP-galactose production with genomic knock-outs of the E. coli ushA and galT genes. The mutant E. coli strains can also optionally be adapted with a genomic knock-in of a constitutive transcriptional unit for a UDP-glucose-4-epimerase like e.g. galE from E. coli (UniProt ID P09147). Furthermore, if the mutant strains were also intended to make sialylated structures, the strains were additionally modified with genomic knock-ins or expression plasmids comprising constitutive transcriptional units for one or more copies of an N-acylneuraminate cytidylyltransferase like e.g. NeuA from P. multocida with SEQ ID NO 61, NeuA from C. jejuni (UniProt ID Q93MP7) or NeuA from H. influenzae with SEQ ID NO 62, and one or more copies of a beta-galactoside alpha-2,3-sialyltransferase like e.g. SEQ ID NO 64 from P. multocida subsp. multocida str. Pm70 and SEQ ID NO 65 (NmeniST3) from N. meningitidis, a beta-galactoside alpha-2,6-sialyltransferase like e.g. PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity and P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity and/or an alpha-2,8-sialyltransferase like e.g. from M. musculus (UniProt ID Q64689). The mutant E. coli strains can also optionally be adapted for growth on sucrose via genomic knock-ins of constitutive transcriptional units containing a sucrose transporter like e.g. CscB from E. coli W (UniProt ID E0IXR1), a fructose kinase like e.g. Frk originating from Z. mobilis (UniProt ID Q03417) and a sucrose phosphorylase like e.g. from B. adolescentis (UniProt ID A0ZZH6).

Preferably but not necessarily, the glycosyltransferases were N- and/or C-terminally fused to a solubility enhancer tag like e.g. a SUMO-tag, an MBP-tag, His, FLAG, Strep-II, Halo-tag, NusA, thioredoxin, GST and/or the Fh8-tag to enhance their solubility (Costa et al., Front. Microbiol. 2014, https://doi.org/10.3389/fmicb.2014.00063; Fox et al., Protein Sci. 2001, 10(3), 622-630; Jia and Jeaon, Open Biol. 2016, 6: 160196).

Optionally, the mutant E. coli strains are modified to create a glycominimized E. coli strain comprising genomic knock-out of any one or more of non-essential glycosyltransferase genes comprising pgaC, pgaD, rfe, rffT, rffM, bcsA, bcsB, bcsC, wcaA, wcaC, wcaE, wcal, wcaJ, wcaL, waaH, waaF, waaC, waaU, waaZ, waaJ, waaO, waaB, waaS, waaG, waaQ, wbbl, arnC, arnT, yfdH, wbbK, opgG, opgH, ycjM, gIgA, glgB, malQ, otsA and yaiP.

All constitutive promoters, UTRs and terminator sequences originated from the libraries described by Mutalik et al. (Nat. Methods 2013, No. 10, 354-360) and Cambray et al. (Nucleic Acids Res. 2013, 41(9), 5139-5148). All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 70% glycerol).

TABLE 1
Overview of polypeptides with corresponding SEQ ID NOs
or UniProt IDs described in the present invention

| | | | | Country of origin of |
|---|---|---|---|---|
| | | | | digital sequence |
| SEQ ID NO | Name / identifier | Organism | Origin | information |
| 01 | nanT (P41036) | E. coli K-12 MG1655 | Synthetic | USA |
| 02 | nanT (A0A0H3NU41) | Yersinia enterocolitica subsp. | Synthetic | Germany |
| | | palearctica serotype O: 3 | | |
| 03 | nanT (A0A1B7IK14) | Buttiauxella brennerae ATCC | Synthetic | Germany |
| | | 51605 | | |
| 04 | nanT (L3PL77) | E. coli KTE75 | Synthetic | Denmark |
| 05 | D4XVE0 | Mycoplasma alligatoris A21JP2 | Synthetic | USA |
| 06 | A0A0C2VH96 | Mycoplasma capricolum subsp. | Synthetic | Germany |
| | | capricolum | | |
| 07 | A0A0D3Q460 | Staphylococcus aureus | Synthetic | USA |
| 08 | A0A533HZC4 | Gemella sp. | Synthetic | USA |
| 09 | A0A125V7I6 | Clostridioides difficile ATCC 9689 | Synthetic | UK |
| 10 | A0A1H8K9Q3 | Peptostreptococcus russellii | Synthetic | USA |
| 11 | A0A1L8ZSQ8 | Bacillus obstructivus | Synthetic | USA |
| 12 | A0A0H3JVD2 | Staphylococcus aureus (strain | Synthetic | USA |
| | | MW2) | | |
| 13 | A0A1S1F7G7 | Staphylococcus sp. | Synthetic | Unknown |
| | | HMSC070A03 | | |
| 14 | A0A1T4NAC5 | Cetobacterium ceti | Synthetic | UK |
| 15 | A0A2K4FCH9 | Staphylococcus argensis | Synthetic | Germany |
| 16 | A0A0M4UEF4 | Staphylococcus schleiferi | Synthetic | Japan |
| 17 | K9EWT1 | Alloiococcus otitis ATCC 51267 | Synthetic | Unknown |
| 18 | A0A317KV18 | Gracilibacillus dipsosauri | Synthetic | USA |
| 19 | A0A0R1S9A9 | Lactobacillus versmoldensis | Synthetic | Germany |
| 20 | A0A556U9X1 | Lactobacillus sp. LL6 | Synthetic | USA |
| 21 | A0A0R1Y239 | Agrilactobacillus composti | Synthetic | Japan |
| 22 | A0A2N5L0P0 | Lactobacillus crispatus | Synthetic | USA |
| 23 | A0A431ZS05 | Staphylococcus pasteuri | Synthetic | Germany |
| 24 | LSCP400_11881 | Lactobacillus salivarius cp400 | Synthetic | The Netherlands |
| 25 | A0A0R1TYU7 | Lactobacillus apodemi | Synthetic | Japan |
| 26 | A0A0K1FGD7 | Selenomonas sp. oral taxon 478 | Synthetic | USA |
| 27 | A0A0C7PUW9 | Paeniclostridium sordellii | Synthetic | UK |
| 28 | A0A1E7RYL9 | Butyribacterium methylotrophicum | Synthetic | Germany |
| 29 | A0A1Q8TWT1 | Alkalihalobacillus pseudofirmus | Synthetic | USA |
| 30 | D3H850 | Streptococcus mitis (strain B6) | Synthetic | Germany |
| 31 | A0A2X0UKU9 | Aerococcus viridans | Synthetic | UK |
| 32 | A0A2X0VQ52 | Anaerobiospirillum thomasii | Synthetic | UK |
| 33 | A0A0A0II53 | Clostridium haemolyticum NCTC | Synthetic | USA |
| | | 8350 | | |
| 34 | A0A1V4SXX4 | Clostridium thermobutyricum | Synthetic | USA |
| 35 | N9WAN6 | Clostridium thermobutyricum | Synthetic | USA |
| 36 | A0A1V1HZG9 | Romboutsia ilealis | Synthetic | The Netherlands |
| 37 | H3KA61 | Megamonas funiformis YIT 11815 | Synthetic | Japan |
| 38 | A0A3P6L5Y9 | Lysinibacillus sphaericus | Synthetic | UK |
| 39 | A0A1C5UWV6 | uncultured Clostridium sp. | Synthetic | UK |
| 40 | B6FWE2 | Clostridium hiranonis | Synthetic | Japan |
| 41 | A0A6M0RCY3 | Clostridium niameyense | Synthetic | Unknown |
| 42 | A0A1B7LK65 | Candidatus Arthromitus sp. SFB- | Synthetic | USA |
| | | turkey | | |
| 43 | siaT_5 (A0A380MYE0) | Suttonella ornithocola | Synthetic | UK |
| 44 | A0A0E3V465 | Pasteurella multocida subsp. | Synthetic | Unknown |
| | | multocida OH4807 | | |
| 45 | A0A1H7ZQG5 | Pasteurella skyensis | Synthetic | UK |
| 46 | A0A1T0B8S6 | [Haemophilus] felis | Synthetic | USA |
| 47 | A0A2G6EKL2 | Gammaproteobacteria bacterium | Synthetic | USA |
| 48 | A0A379B306 | [Pasteurella] mairii | Synthetic | UK |
| 49 | A0A380MWW9 | Suttonella indologenes | Synthetic | The Netherlands |
| 50 | A0A3P1USS0 | Fusobacterium canifelinum | Synthetic | UK |
| 51 | A0A448MCK2 | Fusobacterium varium | Synthetic | USA |
| 52 | A0A4V1QY13 | Fusobacterium necrophorum | Synthetic | USA |
| 53 | C8N6B9 | Cardiobacterium hominis | Synthetic | USA |
| 54 | H0KGY6 | Aggregatibacter | Synthetic | USA |
| | | actinomycetemcomitans RhAA1 | | |
| 55 | I3DJD6 | Pasteurella bettyae CCUG 2042 | Synthetic | UK |
| 56 | B0187_07585 | Haemophilus paracuniculus | Synthetic | USA |
| 57 | B0187_07590 | Haemophilus paracuniculus | Synthetic | USA |
| 58 | B0187_07595 | Haemophilus paracuniculus | Synthetic | USA |
| 59 | B0187_07600 | Haemophilus paracuniculus | Synthetic | USA |
| 60 | HpFutC | Helicobacter pylori UA1234 | Synthetic | United Kingdom |
| 61 | NeuA | Pasteurella multocida | Synthetic | USA |
| 62 | NeuA | Haemophilus influenzae | Synthetic | USA |
| 63 | NANS | Syntrophorhabdus sp. | Synthetic | USA |
| | | PtaU1.Bin058 | | |
| 64 | alpha-2,3-sialyltransferase | Pasteurella multocida subsp. | Synthetic | Unknown |
| | | multocida str. Pm70 | | |
| 65 | alpha-2,3-sialyltransferase | Neisseria meningitidis | Synthetic | United Kingdom |
| 66 | IgtA | Neisseria meningitidis | Synthetic | United Kingdom |

| | | | | Country of origin of |
|---|---|---|---|---|
| | | | | digital sequence |
| UniProt ID | Name / identifier | Organism | Origin | information |
| P43577 | GNA1 | Saccharomyces cerevisiae | Synthetic | USA |
| A7LVG6 | AGE | Bacteroides ovatus | Synthetic | USA |
| E0NCD4 | NeuB | Neisseria meningitidis | Synthetic | United Kingdom |
| none | glmS*54 | Escherichia coli K-12 MG1655 | Synthetic | USA |
| P17169 | glmS | Escherichia coli K-12 MG1655 | Synthetic | USA |
| P31120 | glmM | Escherichia coli K-12 MG1655 | Synthetic | USA |
| P0ACC7 | glmU | Escherichia coli K-12 MG1655 | Synthetic | USA |
| Q93MP8 | NeuC | Campylobacter jejuni | Synthetic | USA |
| Q93MP7 | NeuA | Campylobacter jejuni | Synthetic | USA |
| Q91WG8 | GNE | Mus musculus (strain C57BL/6J) | Synthetic | USA |
| KPA15328.1 | NANP | Candidatus Magnetomorum sp. | Synthetic | Germany |
| | | HK-1 | | |
| Q8A712 | NANP | Bacteroides thetaiotaomicron | Synthetic | Unknown |
| O66375 | alpha-2,6-sialyltransferase | Photobacterium damselae | Synthetic | Japan |
| A8QYL1 | alpha-2,6-sialyltransferase | Photobacterium sp. JT-ISH-224 | Synthetic | Japan |
| Q64689 | alpha-2,8-sialyltransferase | Mus musculus | Synthetic | USA |
| P02920 | LacY | Escherichia coli K-12 MG1655 | Synthetic | USA |
| E0IXR1 | CscB | Escherichia coli W | Synthetic | USA |
| Q03417 | Frk | Zymomonas mobilis | Synthetic | United Kingdom |
| A0ZZH6 | BaSP | Bifidobacterium adolescentis | Synthetic | Germany |
| O30511 | HpFucT | Helicobacter pylori UA1234 | Synthetic | United Kingdom |
| P0A884 | thyA | Escherichia coli K-12 MG1655 | Synthetic | USA |
| P00946 | manA | Escherichia coli K-12 MG1655 | Synthetic | USA |
| P24175 | manB | Escherichia coli K-12 MG1655 | Synthetic | USA |
| P24174 | manC | Escherichia coli K-12 MG1655 | Synthetic | USA |
| P0AC88 | gmd | Escherichia coli K-12 MG1655 | Synthetic | USA |
| P32055 | fcl | Escherichia coli K-12 MG1655 | Synthetic | USA |
| P11551 | fucP | Escherichia coli K-12 MG1655 | Synthetic | USA |
| SUV40286.1 | fkp | Bacteroides fragilis NCTC 9343 | Synthetic | United Kingdom |
| D3QY14 | wbgO | Escherichia coli O55: H7 | Synthetic | Germany |
| Q51116 | lgtB | Neisseria meningitidis MC58 | Synthetic | United Kingdom |
| P09147 | galE | Escherichia coli K-12 MG1655 | Synthetic | USA |
| P07921 | LAC12 | Kluyveromyces lactis | Synthetic | USA |
| Q93MP9 | NeuB | Campylobacter jejuni | Synthetic | USA |
| P23538 | ppsA | Escherichia coli K-12 MG1655 | Synthetic | USA |
| Q6F5A5 | PCK | Corynebacterium glutamicum | Synthetic | Denmark |
| P22259 | pckA | Escherichia coli K-12 MG1655 | Synthetic | USA |
| P0A955 | eda | Escherichia coli K-12 MG1655 | Synthetic | USA |
| P26616 | maeA | Escherichia coli K-12 MG1655 | Synthetic | USA |
| P76558 | maeB | Escherichia coli K-12 MG1655 | Synthetic | USA |
| P27550 | acs | Escherichia coli K-12 MG1655 | Synthetic | USA |
| P0A840 | surE | Escherichia coli K-12 MG1655 | Synthetic | USA |

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96 well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor (having a 5 L working volume) was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2SO4 and 20% NH4OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Analytical Analysis

Standards such as but not limited to sucrose, lactose, N-acetyllactosamine (LacNAc, Gal-b1,4-GlcNAc), lacto-N-biose (LNB, Gal-b1,3-GlcNAc), fucosylated LacNAc (2′FLacNAc, 3-FLacNAc), sialylated LacNAc, (3′SLacNAc, 6′SLacNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analysed with in-house made standards.

Sialylated oligosaccharides were analysed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C. Neutral oligosaccharides were analysed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a % water and % acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C. Both neutral and sialylated sugars were analysed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analysed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Example 5. Materials and Methods Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

### Plasmids

In an example to produce sialic acid and CMP-sialic acid, a yeast expression plasmid can be derived from the pRS420-plasmid series (Christianson et al., 1992, Gene 110: 119-122) containing the TRP1 selection marker and constitutive transcriptional units for one or more sialic acid transporters chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, an L-glutamine-D-fructose-6-phosphate aminotransferase like e.g. the mutant glmS*54 from E. coli (differing from the wild-type E. coli glmS with UniProt ID P17169 by an A39T, an R250C and an G472S mutation), a phosphatase like e.g. the E. coli SurE (UniProt ID P0A840), an N-acylglucosamine 2-epimerase like e.g. AGE from B. ovatus (UniProt ID A7LVG6), an N-acetylneuraminate synthase like e.g. NeuB from N. meningitidis (UniProt ID E0NCD4) or from C. jejuni (UniProt ID Q93MP9) and an N-acylneuraminate cytidylyltransferase like e.g. NeuA from P. multocida with SEQ ID NO 61. Optionally, a constitutive transcriptional unit for a glucosamine 6-phosphate N-acetyltransferase like e.g. GNA1 from S. cerevisiae (UniProt ID P43577) was added as well. To produce sialylated oligosaccharides, the plasmid further comprised constitutive transcriptional units for a lactose permease like e.g. LAC12 from Kluyveromyces lactis (UniProt ID P07921), and one or more sialyltransferases like e.g. a beta-galactoside alpha-2,3-sialyltransferase like e.g. SEQ ID NO 64 from P. multocida subsp. multocida str. Pm70 and SEQ ID NO 65 (NmeniST3) from N. meningitidis, a beta-galactoside alpha-2,6-sialyltransferase like e.g. PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity and P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity and/or an alpha-2,8-sialyltransferase like e.g. from M. musculus (UniProt ID Q64689).

In an example to produce GDP-fucose, a yeast expression plasmid like p2a_2μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2μ yeast on and the Ura3 selection marker for selection and maintenance in yeast. This plasmid is further modified with constitutive transcriptional units for a lactose permease like e.g. LAC12 from K. lactis (UniProt ID P07921), a GDP-mannose 4,6-dehydratase like e.g. gmd from E. coli (UniProt ID P0AC88) and a GDP-L-fucose synthase like e.g. fcl from E. coli (UniProt ID P32055). The yeast expression plasmid p2a_2μ_Fuc2 can be used as an alternative expression plasmid of the p2a_2μ_Fuc plasmid comprising next to the ampicillin resistance gene, the bacterial ori, the 2μ yeast on and the Ura3 selection marker constitutive transcriptional units for a lactose permease like e.g. LAC12 from K. lactis (UniProt ID P07921), a fucose permease like e.g. fucP from E. coli (UniProt ID P11551) and a bifunctional fucose kinase/fucose-1-phosphate guanylyltransferase like e.g. fkp from B. fragilis (UniProt ID SUV40286.1). To further produce fucosylated oligosaccharides, the p2a_2μ_Fuc and its variant the p2a_2μ_Fuc2, additionally contained (a) constitutive transcriptional unit(s) for one or more fucosyltransferases like e.g. an alpha-1,2-fucosyltransferase like e.g. HpFutC from H. pylori with SEQ ID NO 60 and/or an alpha-1,3-fucosyltransferase like e.g. HpFucT from H. pylori (UniProt ID 030511).

In an example to produce UDP-galactose, a yeast expression plasmid can be derived from the pRS420-plasmid series (Christianson et al., 1992, Gene 110: 119-122) containing the HIS3 selection marker and a constitutive transcriptional unit for a UDP-glucose-4-epimerase like e.g. galE from E. coli (UniProt ID P09147). This plasmid was further modified with constitutive transcriptional units for a lactose permease like e.g. LAC12 from K. lactis (UniProt ID P07921), a galactoside beta-1,3-N-acetylglucosaminyltransferase like e.g. IgtA from N. meningitidis with SEQ ID NO 66 to produce LN3. To further produce LN3-derived oligosaccharides like LNT or LNnT, an N-acetylglucosamine beta-1,3-galactosyltransferase like e.g. WbgO from E. coli O55:H7 (UniProt ID D3QY14) or an N-acetylglucosamine beta-1,4-galactosyltransferase like e.g. IgtB from N. meningitidis (UniProt ID Q51116), respectively, was also added on the plasmid.

Proteins used are enlisted in Table 1.

Preferably but not necessarily, the glycosyltransferases and/or the proteins involved in nucleotide-activated sugar synthesis were N- and/or C-terminally fused to a SUMOstar tag (e.g. obtained from pYSUMOstar, Life Sensors, Malvern, PA) to enhance their solubility.

Optionally, the mutant yeast strains were modified with a genomic knock-in of a constitutive transcriptional unit encoding a chaperone protein like e.g. Hsp31, Hsp32, Hsp33, Sno4, Kar2, Ssb1, Sse1, Sse2, Ssa1, Ssa2, Ssa3, Ssa4, Ssb2, Ecm10, Ssc1, Ssg1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5: 275).

Plasmids were maintained in the host E. coli DH5alpha (F⁻, phi80dlacZdeltaM15, delta(lacZYA-argF)U169, deoR, recA1, endA1, hsdR17(rk⁻, mk⁺), phoA, supE44, lambda⁻, thi-1, gyrA96, relA1) bought from Invitrogen.

### Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

### Gene Expression Promoters

Genes were expressed using synthetic constitutive promoters, as described by e.g. Blazeck (Biotechnology and Bioengineering, Vol. 109, No. 11, 2012), Redden and Alper (Nat. Commun. 2015, 6, 7810), Liu et al. (Microb. Cell Fact. 2020, 19, 38), Xu et al. (Microb. Cell Fact. 2021, 20, 148) and Lee et al. (ACS Synth. Biol. 2015, 4(9), 975-986).

### Example 6. Production of Sialic Acid (NeuSAc) with a Modified E. coli Strain

An E. coli K-12 strain MG1655 is modified for sialic acid production as described in Example 4 comprising knock-outs of the E. coli nagA, nagB, nanA, nanT, nanE, nanK, LacZ and LacA genes and genomic knock-ins of constitutive transcriptional units containing a sialic acid transporter chosen from the list comprising SEQ ID NO 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, the L-glutamine-D-fructose-6-phosphate aminotransferase glmS*54 from E. coli (differing from the wild-type E. coli glmS with UniProt ID P17169 by an A39T, an R250C and an G472S mutation), the glucosamine 6-phosphate N-acetyltransferase GNA1 from S. cerevisiae (UniProt ID P43577), the N-acylglucosamine 2-epimerase AGE from B. ovatus (UniProt ID A7LVG6), the N-acetylneuraminate synthase NeuB from N. meningitidis (UniProt ID E0NCD4), the sucrose transporter CscB from E. coli W (UniProt ID E0IXR1), the fructose kinase Frk from Z. mobilis (UniProt ID Q03417) and the sucrose phosphorylase from B. adolescentis (UniProt ID A0ZZH6). The novel strains are evaluated in a growth experiment according to the culture conditions provided in Example 4, in which the culture medium contains 30 g/L sucrose. Each strain is grown in four biological replicates in a 96-well plate. After 72 h of incubation, the culture broth is harvested, and the sugars are analysed on UPLC.

### Example 7. Production of Sialic Acid (NeuSAc) with a Modified E. coli Strain

An E. coli K-12 strain MG1655 is modified for sialic acid production as described in Example 4 comprising knock-outs of the E. coli nagA, nagB, nanA, nanT, nanE, nanK, LacZ and LacA genes and genomic knock-ins of constitutive transcriptional units containing a sialic acid transporter chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, the L-glutamine-D-fructose-6-phosphate aminotransferase glmS*54 from E. coli (differing from the wild-type E. coli glmS with UniProt ID P17169 by an A39T, an R250C and an G472S mutation), the phosphoglucosamine mutase glmM from E. coli (UniProt ID P31120), the bifunctional N-acetylglucosamine-1-phosphate uridyltransferase/glucosamine-1-phosphate acetyltransferase glmU from E. coli (UniProt ID P0ACC7), the bifunctional UDP-GlcNAc 2-epimerase/N-acetylmannosamine kinase from M. musculus (strain C57BL/6J) (UniProt ID Q91WG8), the N-acylneuraminate-9-phosphate synthetase from Syntrophorhabdus sp. PtaU1.Bin058 with SEQ ID NO 63 and an N-acylneuraminate-9-phosphatase from Candidatus Magnetomorum sp. HK-1 (UniProt ID KPA15328.1) and/or from Bacteroides thetaiotaomicron (UniProt ID Q8A712), the sucrose transporter CscB from E. coli W (UniProt ID E0IXR1), the fructose kinase Frk from Z. mobilis (UniProt ID Q03417) and the sucrose phosphorylase from B. adolescentis (UniProt ID A0ZZH6). The novel strains are evaluated in a growth experiment according to the culture conditions provided in Example 4, in which the culture medium contains 30 g/L sucrose. Each strain is grown in four biological replicates in a 96-well plate. After 72 h of incubation, the culture broth is harvested, and the sugars are analysed on UPLC.

### Example 8. Production of Sialic Acid (NeuSAc) with a Modified E. coli Strain

An E. coli K-12 strain MG1655 is modified for sialic acid production as described in Example 4 comprising knock-outs of the E. coli nagA, nagB, nanA, nanT, nanE, nanK, LacZ and LacA genes and genomic knock-ins of constitutive transcriptional units containing a sialic acid transporter chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, the L-glutamine-D-fructose-6-phosphate aminotransferase glmS*54 from E. coli (differing from the wild-type E. coli glmS with UniProt ID P17169 by an A39T, an R250C and an G472S mutation), the phosphoglucosamine mutase glmM from E. coli (UniProt ID P31120), the bifunctional N-acetylglucosamine-1-phosphate uridyltransferase/glucosamine-1-phosphate acetyltransferase glmU from E. coli (UniProt ID P0ACC7), the UDP-N-acetylglucosamine 2-epimerase NeuC from C. jejuni (UniProt ID Q93MP8), the N-acetylneuraminate synthase NeuB from N. meningitidis (UniProt ID E0NCD4), the sucrose transporter CscB from E. coli W (UniProt ID E0IXR1), the fructose kinase Frk from Z. mobilis (UniProt ID Q03417) and the sucrose phosphorylase from B. adolescentis (UniProt ID A0ZZH6). The novel strains are evaluated in a growth experiment according to the culture conditions provided in Example 4, in which the culture medium contains 30 g/L sucrose. Each strain is grown in four biological replicates in a 96-well plate. After 72 h of incubation, the culture broth is harvested, and the sugars are analysed on UPLC.

### Example 9. Production of 6′-Sialyllactose (6′-SL) with a Modified E. coli Strain

An E. coli K-12 strain MG1655 was modified for sialic acid production as described in Example 4 comprising knock-outs of the E. coli nagA, nagB, nanA, nanT, nanE, nanK, LacZ and LacA genes and genomic knock-ins of constitutive transcriptional units containing the sialic acid transporter nanT from E. coli with SEQ ID NO 01, the L-glutamine-D-fructose-6-phosphate aminotransferase glmS*54 from E. coli (differing from the wild-type E. coli glmS with UniProt ID P17169 by an A39T, an R250C and an G472S mutation), the glucosamine 6-phosphate N-acetyltransferase GNA1 from S. cerevisiae (UniProt ID P43577), the N-acylglucosamine 2-epimerase AGE from B. ovatus (UniProt ID A7LVG6), the N-acetylneuraminate synthase NeuB from N. meningitidis (UniProt ID E0NCD4), the sucrose transporter CscB from E. coli W (UniProt ID E0IXR1), the fructose kinase Frk from Z. mobilis (UniProt ID Q03417) and the sucrose phosphorylase from B. adolescentis (UniProt ID A0ZZH6). Herein, three different strains were created that each expressed the E. coli nanT gene with SEQ ID NO 01 from a different transcriptional unit. The three different transcriptional units used for said nanT expression is given in Table 2. In a next step, the three mutant strains were further modified for 6′-SL production comprising a knock-out of the E. coli LacYgene, genomic knock-ins of constitutive transcriptional units containing the lactose permease LacY from E. coli (UniProt ID P02920), the N-acylneuraminate cytidylyltransferases from C. jejuni (UniProt ID Q93MP7) and from H. influenzae with SEQ ID NO 62 and the PdST6-like polypeptide consisting of amino acid residues 108 to 497 of UniProt ID 066375 having beta-galactoside alpha-2,6-sialyltransferase activity as well as an expression plasmid comprising constitutive transcriptional units for the N-acylneuraminate cytidylyltransferase from P. multocida with SEQ ID NO 61 and the PdST6-like polypeptide consisting of amino acid residues 108 to 497 of UniProt ID 066375 having beta-galactoside alpha-2,6-sialyltransferase activity. The three novel 6′SL strains were evaluated in a growth experiment according to the culture conditions provided in Example 4, in which the culture medium contained 30 g/L sucrose and 20 g/L lactose. A reference strain was used lacking the nanT gene with SEQ ID NO 01. Each strain was grown in four biological replicates in a 96-well plate. After 72 h of incubation, the culture broth was harvested, and the sugars were analysed on UPLC. The average production of sialic acid (Neu5Ac) and 6′SL in the mutant strains was normalized to the average sialic acid (Neu5Ac) and 6′SL titres observed in the culture broth for the reference strain. The experiment demonstrated all novel strains produced at the end of cultivation much less Neu5Ac but similar or more 6′SL in whole broth samples than the reference strain lacking nanT (Table 3). The Neu5Ac titres produced in the new strains were also less than 5% of the 6′SL titres produced. Additionally, the Neu5Ac titres produced in the new strains were also less than 5% of the sum of total sugars (Neu5Ac+6′SL) produced in these new strains. Lastly, the genomic knock-in of the nanT sialic acid transporter from E. coli with SEQ ID NO 01 did not alter the growth speed of the mutant strains compared to the reference strain (Results not shown).

TABLE 2
Transcriptional units (TUs) in modified E. coli strains to express
the sialic acid transporter nanT from E. coli with SEQ ID NO 01

| TU | Promoter* | UTR* | Terminator** |
|---|---|---|---|
| TU_01 | PROM0010 = Mutalik_P10 | UTR0044_GalE_ulaEF | TER0007_ilvGEDA |
| TU_02 | PROM0048 = Mutalik_apFAB74 | UTR0008_OmpC_BCD2 | TER0007_ilvGEDA |
| TU_03 | PROM0048 = Mutalik_apFAB74 | UTR0018_GalE_BCD18 | TER0007_ilvGEDA |

*Promoter and UTR sequences are according to Mutalik et al. (Nat. Methods 2013, No. 10, 354-360).
**Terminator sequences are according to Cambray et al. (2013) (Nucleic Acids Res. 2013, 41(9), 5139-5148).

TABLE 3
Relative production of Neu5Ac and 6′-SL (%) in whole broth
samples of a single modified E. coli strain expressing a sialic
acid transporter nanT from E. coli with SEQ ID NO 01 in
a transcriptional unit (TU) according to Table 2, when grown
for 72 hours in minimal medium with sucrose and lactose.

| Strain | TU for nanT expression | Neu5Ac (%) | 6′-SL (%) |
|---|---|---|---|
| SL1 | TU_01 | 0 | 133.4 ± 8.2 |
| SL2 | TU_02 | 30.7 ± 0.1 | 131.0 ± 3.8 |
| SL3 | TU_03 | 24.8 ± 4.2 | 101.4 ± 1.4 |
| Reference strain | none | 100 | 100 |

TABLE 4
Production of Neu5Ac compared to 6′-SL (%) or compared to the
total sum of sugars (Neu5Ac + 6′-SL) produced (%) in whole
broth samples of a single modified E. coli strain expressing
a sialic acid transporter nanT from E. coli with SEQ ID NO
01 in a transcriptional unit (TU) according to Table 2, when
grown for 72 hours in minimal medium with sucrose and lactose.

| Strain | Neu5Ac/6′-SL in % | Neu5Ac/(6′-SL + Neu5Ac) in % |
|---|---|---|
| SL1 | 0 | 0 |
| SL2 | 1.69 ± 0.48 | 1.67 ± 0.47 |
| SL3 | 1.76 ± 0.30 | 1.73 ± 0.28 |
| Reference strain | 7.20 ± 0.50 | 6.72 ± 0.43 |

### Example 10. Production of 6′-Sialyllactose (6′-SL) with a Modified E. coli Strain

In a next example, the mutant E. coli strains as described in Example 6 are further modified for 6′-SL production comprising a knock-out of the E. coli LacY gene, genomic knock-ins of constitutive transcriptional units containing the lactose permease LacY from E. coli (UniProt ID P02920), the N-acylneuraminate cytidylyltransferases from C. jejuni (UniProt ID Q93MP7) and from H. influenzae with SEQ ID NO 62 and the PdST6-like polypeptide consisting of amino acid residues 108 to 497 of UniProt ID 066375 having beta-galactoside alpha-2,6-sialyltransferase activity as well as an expression plasmid comprising constitutive transcriptional units for the N-acylneuraminate cytidylyltransferase from P. multocida with SEQ ID NO 61 and the PdST6-like polypeptide consisting of amino acid residues 108 to 497 of UniProt ID 066375 having beta-galactoside alpha-2,6-sialyltransferase activity. The novel strains are evaluated in a growth experiment according to the culture conditions provided in Example 4, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 11. Production of 3′-Sialyllactose (3′-SL) with a Modified E. coli Strain

An E. coli K-12 strain MG1655 is modified for sialic acid production as described in Example 4 comprising knock-outs of the E. coli nagA, nagB, nanA, nanT, nanE, nanK, LacZ and LacA genes and genomic knock-ins of constitutive transcriptional units containing a sialic acid transporter chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, the L-glutamine-D-fructose-6-phosphate aminotransferase glmS*54 from E. coli (differing from the wild-type E. coli glmS with UniProt ID P17169 by an A39T, an R250C and an G472S mutation), the glucosamine 6-phosphate N-acetyltransferase GNA1 from S. cerevisiae (UniProt ID P43577), the N-acylglucosamine 2-epimerase AGE from B. ovatus (UniProt ID A7LVG6), the N-acetylneuraminate synthase NeuB from N. meningitidis (UniProt ID E0NCD4), the sucrose transporter CscB from E. coli W (UniProt ID E0IXR1), the fructose kinase Frk from Z. mobilis (UniProt ID Q03417) and the sucrose phosphorylase from B. adolescentis (UniProt ID A0ZZH6). In a next step, the mutant strains are further modified for 3′-SL production comprising a knock-out of the E. coli LacY gene, genomic knock-ins of constitutive transcriptional units containing the lactose permease LacY from E. coli (UniProt ID P02920), the N-acylneuraminate cytidylyltransferases from C. jejuni (UniProt ID Q93MP7) and from H. influenzae with SEQ ID NO 62 and a beta-galactoside alpha-2,3-sialyltransferase from P. multocida subsp. multocida str. Pm70 with SEQ ID NO 64 or from N. meningitidis with SEQ ID NO 65 as well as an expression plasmid comprising constitutive transcriptional units for the N-acylneuraminate cytidylyltransferase from P. multocida with SEQ ID NO 61 and a beta-galactoside alpha-2,3-sialyltransferase from P. multocida subsp. multocida str. Pm70 with SEQ ID NO 64 or from N. meningitidis with SEQ ID NO 65. The novel strains are evaluated in a growth experiment according to the culture conditions provided in Example 4, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 12. Evaluation of a Modified E. coli Strain in Fed-Batch Fermentations

An E. coli K-12 strain MG1655 was modified for sialic acid production as described in Example 4 comprising knock-outs of the E. coli nagA, nagB, nanA, nanT, nanE, nanK, LacZ and LacA genes and genomic knock-ins of constitutive transcriptional units containing the sialic acid transporter nanT from E. coli with SEQ ID NO 01, the L-glutamine-D-fructose-6-phosphate aminotransferase glmS*54 from E. coli (differing from the wild-type E. coli glmS with UniProt ID P17169 by an A39T, an R250C and an G472S mutation), the glucosamine 6-phosphate N-acetyltransferase GNA1 from S. cerevisiae (UniProt ID P43577), the N-acylglucosamine 2-epimerase AGE from B. ovatus (UniProt ID A7LVG6), the N-acetylneuraminate synthase NeuB from C. jejuni (UniProt ID Q93MP9), the sucrose transporter CscB from E. coli W (UniProt ID E0IXR1), the fructose kinase Frk from Z. mobilis (UniProt ID Q03417) and the sucrose phosphorylase from B. adolescentis (UniProt ID A0ZZH6). The nanT sialic acid transporter from E. coli with SEQ ID NO 01 was presented to the strain in a transcriptional unit comprising promoter sequence PROM0010_Mutalik_P10 (Mutalik et al., Nat. Methods 2013, No. 10, 354-360), UTR sequence UTR0044_GalE_ulaEF (Mutalik et al., Nat. Methods 2013, No. 10, 354-360) and terminator sequence TER0007_ilvGEDA (Cambray et al., Nucleic Acids Res. 2013, 41(9), 5139-5148). In a next step, the mutant strain was further modified for 6′-SL production comprising a knock-out of the E. coli LacY gene, genomic knock-ins of constitutive transcriptional units containing the lactose permease LacY from E. coli (UniProt ID P02920), the N-acylneuraminate cytidylyltransferases from C. jejuni (UniProt ID Q93MP7) and from H. influenzae with SEQ ID NO 62 and the PdST6-like polypeptide consisting of amino acid residues 108 to 497 of UniProt ID 066375 having beta-galactoside alpha-2,6-sialyltransferase activity as well as an expression plasmid comprising constitutive transcriptional units for the N-acylneuraminate cytidylyltransferase from P. multocida with SEQ ID NO 61 and the PdST6-like polypeptide consisting of amino acid residues 108 to 497 of UniProt ID 066375 having beta-galactoside alpha-2,6-sialyltransferase activity. The mutant E. coli strain was evaluated in a fed-batch fermentation process. Fed-batch fermentations at bioreactor scale were performed as described in Example 4. A reference E. coli strain lacking nanT with SEQ ID NO 01 was also evaluated. Sucrose was used as a carbon source and lactose was added in the batch medium as a precursor. No sialic acid (Neu5Ac) was added to the fermentation process. In contrast to the cultivation experiments that are described herein and wherein only end samples were taken at the end of cultivation (i.e. 72 hours as described herein), regular broth samples were taken at several time points during the fermentation process and the production of sialic acid (Neu5Ac) and 6′-sialyllactose at each of said time points was measured using UPLC as described in Example 4. The experiment demonstrated that broth samples taken e.g. at the end of the batch phase and during fed-batch phase comprised sialic acid production together with 6′-sialyllactose and unmodified lactose. At the end of the fed-batch phase, the novel mutant strain produced much less sialic acid (Neu5Ac) and higher amounts of 6′-SL compared to the reference strain (Table 5). The mutant strain had also a lower Neu5Ac to 6′-SL ratio (Neu5Ac/6′-SL) compared to the reference strain. The mutant strain had also a lower ratio of Neu5Ac produced on the total amount of 6′-SL and Neu5Ac produced (Neu5Ac/(6′-SL+Neu5Ac)) compared to the reference strain.

TABLE 5
Parameters obtained in fed-batch fermentation

| Strain | Reference strain | Mutant strain |
|---|---|---|
| Neu5Ac (%) at end fed-batch phase | 100 | 37.00 ± 0.34 |
| 6′-SL (%) at end fed-batch phase | 100 | 102.90 ± 0.20 |
| Neu5Ac/6′-SL (%) at end fed-batch phase | 1.55 ± 1.10 | 0.54 ± 0.33 |
| Neu5Ac/(6′-SL + Neu5′Ac) (%) at end fed-batch phase | 1.52 ± 1.08 | 0.54 ± 0.33 |

### Example 13. Evaluation of a Mutant E. coli Strain in Fed-Batch Fermentations

The mutant E. coli strains as described in Example 11 are further evaluated in a fed-batch fermentation process. Fed-batch fermentations at bioreactor scale are performed as described in Example 4. A reference strain is used with identical genetic make-up but lacking a sialic acid transporter. In these examples, sucrose is used as a carbon source and lactose is added in the batch medium as a precursor. No sialic acid (Neu5Ac) is added to the fermentation process. In contrast to the cultivation experiments that are described herein and wherein only end samples are taken at the end of cultivation (i.e. 72 hours as described herein), regular broth samples are taken at several time points during the fermentation process and the production of sialic acid (Neu5Ac) and 3′-sialyllactose is measured at each of said time points using UPLC as described in Example 4.

### Example 14. Production of an Oligosaccharide Mixture Comprising 6′SL, LN3, Sialylated LN3. LNnT and LSTc with a Modified E. coli Host

An E. coli host modified for sialic acid production (Neu5Ac) and 6′-siayllactose as described in Example 9 is further modified with genomic knock-ins comprising constitutive transcriptional units of the galactoside beta-1,3-N-acetylglucosaminyltransferase LgtA from N. meningitidis with SEQ ID NO 66 for production of LN3 and of the N-acetylglucosamine beta-1,4-galactosyltransferase LgtB from N. meningitidis (UniProt ID Q51116) to produce LNnT, as described in Example 4, to produce a mixture of oligosaccharides comprising 6′SL, LN3, LNnT and LSTc (Neu5Ac-a2,6-Gal-b1,4-GlcNAc-b1,3-Gal-b1,4-Glc). The novel strain is evaluated in a growth experiment according to the culture conditions provided in Example 4, in which the culture medium contains sucrose and lactose. The strain is grown in four biological replicates in a 96-well plate. After 72 h of incubation, the culture broth is harvested, and the sugars are analysed on UPLC.

### Example 15. Production of 6′-Sialyllactose (6′-SL) with a Modified E. coli Strain

In a next example, the mutant E. coli strains as described in Examples 7 and 8 are further modified for 6′-SL production comprising a knock-out of the E. coli LacY gene, genomic knock-ins of constitutive transcriptional units containing the lactose permease LacY from E. coli (UniProt ID P02920), the N-acylneuraminate cytidylyltransferases from C. jejuni (UniProt ID Q93MP7) and from H. influenzae with SEQ ID NO 62 and the PdST6-like polypeptide consisting of amino acid residues 108 to 497 of UniProt ID 066375 having beta-galactoside alpha-2,6-sialyltransferase activity as well as an expression plasmid comprising constitutive transcriptional units for the N-acylneuraminate cytidylyltransferase from P. multocida with SEQ ID NO 61 and the PdST6-like polypeptide consisting of amino acid residues 108 to 497 of UniProt ID 066375 having beta-galactoside alpha-2,6-sialyltransferase activity. The novel strains are evaluated in a growth experiment according to the culture conditions provided in Example 4, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 16. Production of Sialic Acid (NeuSAc) with a Modified S. cerevisiae Strain

An S. cerevisiae strain is adapted for sialic acid (Neu5Ac) production as described in Example 5 with a pRS420-derived yeast expression plasmid comprising the TRP1 selection marker and constitutive transcriptional units for a sialic acid transporter chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, the mutant glmS*54 from E. coli (differing from the wild-type E. coli glmS with UniProt ID P17169 by an A39T, an R250C and an G472S mutation), the phosphatase SurE from E. coli (UniProt ID P0A840), the N-acylglucosamine 2-epimerase AGE from B. ovatus (UniProt ID A7LVG6) and the N-acetylneuraminate synthase NeuB from N. meningitidis (UniProt ID E0NCD4). The novel strains are evaluated in a growth experiment on SD CSM-Trp drop-out medium according to the culture conditions provided in Example 5. After 72 h of incubation, the culture broth is harvested, and the sugars are analysed on UPLC.

### Example 17. Production of 6′-Sialyllactose (6′-SL) with a Modified S. cerevisiae Strain

An S. cerevisiae strain is adapted for sialic acid (Neu5Ac) and 6′-SL production as described in Example 5 with a pRS420-derived yeast expression plasmid comprising the TRP1 selection marker and constitutive transcriptional units for a sialic acid transporter chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 or 59, the mutant glmS*54 from E. coli (differing from the wild-type E. coli glmS with UniProt ID P17169 by an A39T, an R250C and an G472S mutation), the phosphatase SurE from E. coli (UniProt ID P0A840), the N-acylglucosamine 2-epimerase AGE from B. ovatus (UniProt ID A7LVG6), the N-acetylneuraminate synthase NeuB from N. meningitidis (UniProt ID E0NCD4), the N-acylneuraminate cytidylyltransferase NeuA from P. multocida with SEQ ID NO 61, the PdST6-like polypeptide consisting of amino acid residues 108 to 497 of UniProt ID 066375 having beta-galactoside alpha-2,6-sialyltransferase activity and the lactose permease LAC12 from K. lactis (UniProt ID P07921). The novel strains are evaluated in a growth experiment on SD CSM-Trp drop-out medium according to the culture conditions provided in Example 5. After 72 h of incubation, the culture broth is harvested, and the sugars are analysed on UPLC.

### Example 18. Production of an Oligosaccharide Mixture Comprising 6′SL, LN3, Sialylated LN3. LNnT and LSTc with a Modified S. cerevisiae Host

The mutant S. cerevisiae strains described in Example 17 are further modified with a second pRS420-derived yeast expression plasmid comprising the HIS3 selection marker and constitutive transcriptional units for the UDP-glucose-4-epimerase galE from E. coli (UniProt ID P09147), the galactoside beta-1,3-N-acetylglucosaminyltransferase IgtA from N. meningitidis with SEQ ID NO 66 and the N-acetylglucosamine beta-1,4-galactosyltransferase IgtB from N. meningitidis (UniProt ID Q51116) to produce a mixture of oligosaccharides comprising 6′SL, LN3, LNnT and LSTc (Neu5Ac-a2,6-Gal-b1,4-GlcNAc-b1,3-Gal-b1,4-Glc). The novel strains are evaluated in a growth experiment on SD CSM-Trp-His drop-out medium comprising lactose as precursor according to the culture conditions provided in Example 5. After 72 h of incubation, the culture broth is harvested, and the sugars are analysed on UPLC.

1.-53. (canceled)

54. A metabolically engineered cell of a microorganism for production of a sialylated di- and/or oligosaccharide, the cell comprising a pathway for production of the sialylated di- and/or oligosaccharide, wherein the cell:
synthesizes sialic acid,
expresses at least one sialyltransferase, optionally is modified in the expression or activity of at least one sialyltransferase,
is modified to have a fully or partially knocked out or rendered less functional sialic acid catabolic pathway,
is modified for overexpression of an endogenous sialic acid transporter and/or expression of an exogenous sialic acid transporter, and
optionally, is modified in the expression or activity of at least one sialyltransferase.

55. The cell of claim 54, wherein the pathway for production of the sialylated di- and/or oligosaccharide comprises a sialylation pathway.

56. The cell of claim 55, wherein the sialylation pathway comprises a sialyltransferase combined with at least one enzyme selected from the group consisting of N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthetase, phosphatase, N-acetylneuraminate synthase and N-acylneuraminate cytidylyltransferase, and, optionally, wherein the cell is modified in the expression or activity of at least one of the enzymes.

57. The cell of claim 54, wherein the sialyltransferase is selected from the group consisting of alpha-2,3-sialyltransferase, alpha-2,6-sialyltransferase, and alpha-2,8-sialyltransferase.

58. The cell of claim 54, wherein the sialic acid transporter is selected from the group of porters and P—P-bond-hydrolysis-driven transporters, wherein
a) when the sialic acid transporter is selected from the group of porters, the sialic acid transporter is selected from
the group of eggnog families 1MU0F, 1MUNB, 1MVHH, 1MWKH, 1R4VK, 1RJ8K, 1RMH7, 1RNAM, 1RPTE, 1RQFS, 1RZ3Z, 1SA02, 1TPNU, 1TPVE, 1TQCK, 1XTIW, 1XU43, 1Y733, 1Y7P0, 1YAT3, 258Y6, 25E9U, 25SBT, 25Y42, 26G88, 27G1B, 2IX6M, 2TQBC, 2TQW5, 36UQA, 3788V, 3F50Z, 3WG4Q, 3WP13, 3WTAI, 3WVF7, 3X1B1, 3XP7A, 3ZJ4T, 404JV, 41EHH, 4BDTU, 4C594, 4GX8Z, 4GY09, 4H21M, 4H9KW, 4H9WK, 4HB5K, 4IEK0, COG0477, COG0591, COG1593, COG2704, COG2814, COG3055, COG3090, and COG4666,
the PFAM list PF00083, PF00474, PF04290, PF06808, PF07690, and PF11874,
the interpro list IPR001734, IPR004681, IPR004742, IPR005828, IPR005829, IPR007387, IPR010656, IPR011701, IPR011851, IPR011853, IPR015915, IPR018212, IPR020846, IPR021814, IPR025966, IPR036259, and IPR038377, or
NanT from Escherichia coli with SEQ ID NO: 1, nanT from Yersinia enterocolitica subsp. palearctica serotype O:3 with SEQ ID NO: 2, nanT from Buttiauxella brennerae ATCC 51605 with SEQ ID NO: 3, nanT from E. coli KTE75 with SEQ ID NO: 4, or a transporter from Mycoplasma alligatoris A21JP2 with SEQ ID NO: 5, from M. capricolum subsp. capricolum with SEQ ID NO: 6, from Staphylococcus aureus with SEQ ID NO: 7, from Gemella sp. with SEQ ID NO: 8, from Clostridioides difficile ATCC 9689 with SEQ ID NO: 9, from Peptostreptococcus russellii with SEQ ID NO: 10, from Bacillus obstructivus with SEQ ID NO: 11, from S. aureus (strain MW2) with SEQ ID NO: 12, from Staphylococcus sp. HMSC070A03 with SEQ ID NO: 13, from Cetobacterium ceti with SEQ ID NO: 14, from S. argensis with SEQ ID NO: 15, from S. schleiferi with SEQ ID NO: 16, from Alloiococcus otitis ATCC 51267 with SEQ ID NO: 17, from Gracilibacillus dipsosauri with SEQ ID NO: 18, from Lactobacillus versmoldensis with SEQ ID NO: 19, from Lactobacillus sp. LL6 with SEQ ID NO: 20, from Agrilactobacillus composti with SEQ ID NO: 21, from L. crispatus with SEQ ID NO: 22, from S. pasteuri with SEQ ID NO: 23, from L. salivarius cp400 with SEQ ID NO: 24, from L. apodemi with SEQ ID NO: 25, from Selenomonas sp. oral taxon 478 with SEQ ID NO: 26, from Paeniclostridium sordellii with SEQ ID NO: 27, from Butyribacterium methylotrophicum with SEQ ID NO: 28, from Alkalihalobacillus pseudofirmus with SEQ ID NO: 29, from Streptococcus mitis (strain B6) with SEQ ID NO: 30, from Aerococcus viridans with SEQ ID NO: 31, from Anaerobiospirillum thomasii with SEQ ID NO: 32, from Clostridium haemolyticum NCTC 8350 with SEQ ID NO: 33, from C. thermobutyricum with SEQ ID NO: 34 or SEQ ID NO: 35, from Romboutsia ilealis with SEQ ID NO: 36, from Megamonas funiformis YIT 11815 with SEQ ID NO: 37, from Lysinibacillus sphaericus with SEQ ID NO: 38, from uncultured Clostridium sp. with SEQ ID NO: 39, from C. hiranonis with SEQ ID NO: 40, from C. niameyense with SEQ ID NO: 41, from Candidatus Arthromitus sp. SFB-turkey with SEQ ID NO: 42, siaT_5 from Suttonella ornithocola with SEQ ID NO: 43, a transporter from Pasteurella multocida subsp. multocida OH4807 with SEQ ID NO: 44, from P. skyensis with SEQ ID NO: 45, from Haemophilus felis with SEQ ID NO: 46, from Gammaproteobacteria bacterium with SEQ ID NO: 47, from Pasteurella mairii with SEQ ID NO: 48, from Suttonella indologenes with SEQ ID NO: 49, from Fusobacterium canifelinum with SEQ ID NO: 50, from F. varium with SEQ ID NO: 51, from F. necrophorum with SEQ ID NO: 52, from Cardiobacterium hominis with SEQ ID NO: 53, from Aggregatibacter actinomycetemcomitans RhAA1 with SEQ ID NO: 54, or from P. bettyae CCUG 2042 with SEQ ID NO: 55, or functional homolog, variant or derivative of any one of the SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 having at least 80% overall sequence identity to the full-length sequence of any one of the polypeptides with SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 respectively and having sialic acid transporter activity;
b) when the sialic acid transporter is a P—P-bond-hydrolysis-driven transporter, the sialic acid transporter is selected from
the group of eggnog families 1MU8Z, 1MUZH, 1NU4K, 1R4 KB, 1RNFB, 1RS1R, 1SKPD, 1SMBI, 1Y72G, 1Y72H, 1Y7EZ, 1Y7FQ, COG0444, COG0601, COG0747, COG1173 and COG4608,
the PFAM list PF00005, PF00496, PF00528, and PF08352,
the interpro list IPR000515, IPR000914, IPR003439, IPR003593, IPR006311, IPR013563, IPR017871, IPR027417, IPR030678, IPR035906 and IPR039424, or
ABC transporter from Haemophilus paracuniculus with SEQ ID NO: 56, 57, 58 or 59, or functional homolog, variant or derivative of any one of the SEQ ID NOs 56, 57, 58 or 59 having at least 80% overall sequence identity to the full-length sequence of any one of the polypeptides with SEQ ID NOs 56, 57, 58 or 59, respectively and having sialic acid transporter activity;
wherein the eggnog families are as defined by eggnogdb 5.0.0 as released on November 2018, the PFAM lists are as defined by Pfam 32.0 as released on September 2018, the interpro lists are as defined by InterPro 75.0 as released on 4 July 2019.

59. The cell of claim 54, wherein the sialic acid transporter is selected from the group of transporters consisting of: a) the porters NanT from Escherichia coli with SEQ ID NO: 1, nanT from Yersinia enterocolitica subsp. palearctica serotype O:3 with SEQ ID NO: 2, nanT from Buttiauxella brennerae ATCC 51605 with SEQ ID NO: 3, nanT from E. coli KTE75 with SEQ ID NO: 4, or a transporter from Mycoplasma alligatoris A21JP2 with SEQ ID NO: 5, from M. capricolum subsp. capricolum with SEQ ID NO: 6, from Staphylococcus aureus with SEQ ID NO: 7, from Gemella sp. with SEQ ID NO: 8, from Clostridioides difficile ATCC 9689 with SEQ ID NO: 9, from Peptostreptococcus russellii with SEQ ID NO: 10, from Bacillus obstructivus with SEQ ID NO: 11, from S. aureus (strain MW2) with SEQ ID NO: 12, from Staphylococcus sp. HMSC070A03 with SEQ ID NO: 13, from Cetobacterium ceti with SEQ ID NO: 14, from S. argensis with SEQ ID NO: 15, from S. schleiferi with SEQ ID NO: 16, from Alloiococcus otitis ATCC 51267 with SEQ ID NO: 17, from Gracilibacillus dipsosauri with SEQ ID NO: 18, from Lactobacillus versmoldensis with SEQ ID NO: 19, from Lactobacillus sp. LL6 with SEQ ID NO: 20, from Agrilactobacillus composti with SEQ ID NO: 21, from L. crispatus with SEQ ID NO: 22, from S. pasteuri with SEQ ID NO: 23, from L. salivarius cp400 with SEQ ID NO: 24, from L. apodemi with SEQ ID NO: 25, from Selenomonas sp. oral taxon 478 with SEQ ID NO: 26, from Paeniclostridium sordellii with SEQ ID NO: 27, from Butyribacterium methylotrophicum with SEQ ID NO: 28, from Alkalihalobacillus pseudofirmus with SEQ ID NO: 29, from Streptococcus mitis (strain B6) with SEQ ID NO: 30, from Aerococcus viridans with SEQ ID NO: 31, from Anaerobiospirillum thomasii with SEQ ID NO: 32, from Clostridium haemolyticum NCTC 8350 with SEQ ID NO: 33, from C. thermobutyricum with SEQ ID NO: 34 or SEQ ID NO: 35, from Romboutsia ilealis with SEQ ID NO: 36, from Megamonas funiformis YIT 11815 with SEQ ID NO: 37, from Lysinibacillus sphaericus with SEQ ID NO: 38, from uncultured Clostridium sp. with SEQ ID NO: 39, from C. hiranonis with SEQ ID NO: 40, from C. niameyense with SEQ ID NO: 41, from Candidatus Arthromitus sp. SFB-turkey with SEQ ID NO: 42, siaT_5 from Suttonella ornithocola with SEQ ID NO: 43, a transporter from Pasteurella multocida subsp. multocida OH4807 with SEQ ID NO: 44, from P. skyensis with SEQ ID NO: 45, from [Haemophilus] felis with SEQ ID NO: 46, from Gammaproteobacteria bacterium with SEQ ID NO: 47, from [Pasteurella] mairii with SEQ ID NO: 48, from Suttonella indologenes with SEQ ID NO: 49, from Fusobacterium canifelinum with SEQ ID NO: 50, from F. varium with SEQ ID NO: 51, from F. necrophorum with SEQ ID NO: 52, from Cardiobacterium hominis with SEQ ID NO: 53, from Aggregatibacter actinomycetemcomitans RhAA1 with SEQ ID NO: 54, or from P. bettyae CCUG 2042 with SEQ ID NO: 55 or functional homolog, variant or derivative of any one of the SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 having at least 80% overall sequence identity to the full-length sequence of any one of the polypeptides with SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 respectively and having sialic acid transporter activity; and
b) the P—P-bond-hydrolysis-driven transporters from Haemophilus paracuniculus with SEQ ID NO: 56, 57, 58 or 59, or functional homolog, variant or derivative of any one of the SEQ ID NOs 56, 57, 58 or 59 having at least 80% overall sequence identity to the full-length sequence of any one of the polypeptides with SEQ ID NOs 56, 57, 58 or 59, respectively and having sialic acid transporter activity.

60. The cell of claim 54, wherein the sialic acid transporter is a transporter protein involved in the influx and/or efflux of sialic acid across the cytoplasmic membrane, optionally in and/or out of the cell.

61. The cell of claim 54, wherein any one of the sialic acid transporters and/or enzymes involved in the pathway for production of the sialylated di- and/or oligosaccharide is presented to the cell in one or more gene expression modules wherein expression is regulated by one or more regulatory sequences.

62. The cell of claim 61, wherein the expression modules are integrated in the host cell's genome and/or presented to the cell on a vector comprising plasmid, cosmid, phage, liposome or virus, which is to be stably transformed into the host cell.

63. The cell of claim 54, wherein the cell is capable of synthesizing N-acetylmannosamine (ManNAc), N-acetylmannosamine-6-phosphate (ManNAc-6-phosphate) and/or phosphoenolpyruvate (PEP).

64. The cell of claim 54, wherein the cell is modified for enhanced synthesis and/or supply of phosphoenolpyruvate (PEP).

65. The cell of claim 54, wherein the cell is further capable of synthesizing at least one nucleotide-activated sugar.

66. The cell of claim 65, wherein the nucleotide-activated sugar is selected from the group consisting of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2, CMP-N-glycolylneuraminic acid (CMP-Neu5Gc), GDP-fucose (GDP-Fuc), GDP-rhamnose and UDP-xylose.

67. The cell of claim 65, wherein the nucleotide-activated sugar is synthesized by at least one enzyme selected from the group consisting of mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, N-acetylmannosamine-6-phosphate 2-epimerase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphatase, CMP-sialic acid synthetase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-GalNAc pyrophosphorylase.

68. The cell of claim 54, wherein the cell further expresses a glycosyltransferase selected from the group consisting of fucosyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases, and fucosaminyltransferases.

69. The cell of claim 54, wherein the cell comprises a fucosylation pathway comprising at least one enzyme selected from the group consisting of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-fucokinase/GDP-fucose pyrophosphorylase, and fucosyltransferase.

70. The cell of claim 54, wherein the cell comprises a galactosylation pathway comprising at least one enzyme selected from the group consisting of galactose-1-epimerase, galactokinase, glucokinase, galactose-1-phosphate uridylyltransferase, UDP-glucose 4-epimerase, glucose-1-phosphate uridylyltransferase, phosphoglucomutase, and galactosyltransferase.

71. The cell of claim 54, wherein the cell comprises an N-acetylglucosaminylation pathway comprising at least one enzyme selected from the group consisting of L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase.

72. The cell of claim 54, wherein the cell comprises a modification for reduced production of acetate.

73. The cell of claim 54, wherein the cell further comprises a lower or reduced expression and/or abolished, impaired, reduced or delayed activity of at least one of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, ion protease, glucose-specific translocating phosphotransferase enzyme IIBC component ptsG, glucose-specific translocating phosphotransferase (PTS) enzyme IIBC component malX, enzyme IIAGlc, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, and pyruvate decarboxylase.

74. The cell of claim 54, wherein the cell comprises a catabolic pathway for selected mono-, di- or oligosaccharides which is at least partially inactivated, the mono-, di-, or oligosaccharides being involved in and/or required for the synthesis of the sialylated di- and/or oligosaccharide.

75. The cell of claim 54, wherein the cell utilizes a precursor for the synthesis of the sialylated di- and/or oligosaccharide, the precursor being fed to the cell from the cultivation medium.

76. The cell of claim 54, wherein the cell produces a precursor for the synthesis of the sialylated di- and/or oligosaccharide.

77. The cell of claim 54, wherein the cell produces 90 g/L or more of the sialylated di- and/or oligosaccharide in the whole broth and/or supernatant and/or wherein the sialylated di- and/or oligosaccharide in the whole broth and/or supernatant has a purity of at least 80% measured on the total amount of sialylated di- and/or oligosaccharide and its precursor produced by the cell in the whole broth and/or supernatant, respectively.

78. The cell of claim 54, wherein the sialylated di- and/or oligosaccharide is selected from the group consisting of a milk oligosaccharide, O-antigen, an oligosaccharide repeat present in capsular polysaccharides, peptidoglycan, amino-sugars and Lewis-type antigen oligosaccharides, mammalian milk oligosaccharide, and human milk oligosaccharide.

79. The cell of claim 54, wherein the cell is a bacterium, fungus, or yeast.

80. The cell of claim 54, wherein the cell is stably cultured in a medium.

81. The cell of claim 54, wherein the cell is capable of synthesizing a mixture of oligosaccharides comprising at least one sialylated oligosaccharide.

82. The cell of claim 54, wherein the cell is capable of synthesizing a mixture of di- and oligosaccharides comprising at least one sialylated di- and/or oligosaccharide.

83. A method of producing a sialylated di- and/or oligosaccharide by a cell of a microorganism, the method comprising the steps of:
a) providing the cell of claim 54,
b) cultivating the cell under conditions permissive to produce the sialylated di- and/or oligosaccharide, and
c) optionally separating the sialylated di- and/or oligosaccharide from the cultivation.

84. The method according to claim 83, wherein >95% of the sialic acid synthesized by the cell is used for producing the sialylated di- and/or oligosaccharide.

85. The method according to claim 83, the method further comprising at least one of the following steps:
using a culture medium comprising at least one precursor and/or acceptor for the production of the sialylated di- and/or oligosaccharide, and/or
adding to the culture medium at least one precursor and/or acceptor feed for the production of the sialylated di- and/or oligosaccharide.

86. The method according to claim 83, the method further comprising at least one of the following steps:
using a culture medium comprising at least one precursor and/or acceptor;
adding to the culture medium in a reactor at least one precursor and/or acceptor feed wherein the total reactor volume ranges from 250 mL (milliliter) to 10,000 m³, optionally in a continuous manner, and optionally so that the final volume of the culture medium is not more than three-fold of the volume of the culture medium before the addition of the precursor and/or acceptor feed;
adding to the culture medium in a reactor at least one precursor and/or acceptor feed wherein the total reactor volume ranges from 250 mL (milliliter) to 10,000 m³ optionally in a continuous manner, and optionally so that the final volume of the culture medium is not more than three-fold of the volume of the culture medium before the addition of the precursor and/or acceptor feed and wherein optionally the pH of the precursor and/or acceptor feed is set between 2.0 and 10.0 and wherein optionally, the temperature of the precursor and/or acceptor feed is kept between 20° C. and 80° C.;
adding at least one precursor and/or acceptor feed in a continuous manner to the culture medium over the course of 1 day, 2 days, 3 days, 4 days, 5 days by means of a feeding solution; and
adding at least one precursor and/or acceptor feed in a continuous manner to the culture medium over the course of 1 day, 2 days, 3 days, 4 days, 5 days by means of a feeding solution and wherein optionally the pH of the precursor and/or acceptor feed is set between 2.0 and 10.0 and wherein optionally, the temperature of the feeding solution is kept between 20° C. and 80° C.;
the method resulting in a sialylated di- and/or oligosaccharide with a concentration of at least 50 g/L in the final volume of the culture medium.

87. The method according to claim 83, the method further comprising at least one of the following steps:
using a culture medium comprising at least 50 grams of precursor per liter of initial reactor volume wherein the total reactor volume ranges from 250 mL to 10,000 m³;
using a culture medium comprising at least 50 grams of acceptor per liter of initial reactor volume wherein the total reactor volume ranges from 250 mL to 10,000 m³;
adding to the culture medium in a reactor a precursor feed comprising at least 50 grams of precursor per liter of initial reactor volume wherein the total reactor volume ranges from 250 mL to 10,000 m³ optionally in a continuous manner, and optionally so that the final volume of the culture medium is not more than three-fold of the volume of the culture medium before the addition of the precursor feed;
adding to the culture medium in a reactor an acceptor feed comprising at least 50 grams of acceptor per liter of initial reactor volume wherein the total reactor volume ranges from 250 mL to 10,000 m³ optionally in a continuous manner, and optionally so that the final volume of the culture medium is not more than three-fold of the volume of the culture medium before the addition of the acceptor feed;
adding to the culture medium in a reactor a precursor feed comprising at least 50 grams of precursor per liter of initial reactor volume wherein the total reactor volume ranges from 250 mL to 10,000 m³ optionally in a continuous manner, and optionally so that the final volume of the culture medium is not more than three-fold of the volume of the culture medium before the addition of the precursor feed and wherein optionally the pH of the precursor feed is set between 2.0 and 10.0 and wherein optionally, the temperature of the precursor feed is kept between 20° C. and 80° C.;
adding to the culture medium in a reactor an acceptor feed comprising at least 50 grams of acceptor per liter of initial reactor volume wherein the total reactor volume ranges from 250 mL to 10,000 m³ optionally in a continuous manner, and optionally so that the final volume of the culture medium is not more than three-fold of the volume of the culture medium before the addition of the acceptor feed and wherein optionally the pH of the acceptor feed is set between 2.0 and 10.0 and wherein optionally, the temperature of the acceptor feed is kept between 20° C. and 80° C.;
adding a precursor and/or acceptor feed in a continuous manner to the culture medium over the course of 1 day, 2 days, 3 days, 4 days, 5 days by means of a feeding solution;
adding a precursor feed in a continuous manner to the culture medium over the course of 1 day, 2 days, 3 days, 4 days, 5 days by means of a precursor feeding solution and wherein the concentration of the precursor feeding solution is 50 g/L and wherein optionally the pH of the precursor feed is set between 2.0 and 10.0 and wherein optionally, the temperature of the precursor feed is kept between 20° C. and 80° C.; and
adding an acceptor feed in a continuous manner to the culture medium over the course of 1 day, 2 days, 3 days, 4 days, 5 days by means of an acceptor feeding solution and wherein the concentration of the acceptor feeding solution is 50 g/L and wherein optionally the pH of the acceptor feed is set between 2.0 and 10.0 and wherein optionally, the temperature of the acceptor feed is kept between 20° C. and 80° C.;
the method resulting in a sialylated di- and/or oligosaccharide with a concentration of at least 50 g/L in the final volume of the culture medium.

88. The method according to claim 83, the method further comprising at least one of the following steps:
using a culture medium comprising at least 50 grams of lactose per liter of initial reactor volume wherein the total reactor volume ranges from 250 mL to 10,000 m³;
adding to the culture medium in a reactor a lactose feed comprising at least 50 gram of lactose per liter of initial reactor volume wherein the reactor volume ranges from 250 mL to 10,000 m³ optionally in a continuous manner, and optionally so that the final volume of the culture medium is not more than three-fold of the volume of the culture medium before the addition of the lactose feed;
adding to the culture medium in a reactor a lactose feed comprising at least 50 grams of lactose per liter of initial reactor volume wherein the total reactor volume ranges from 250 mL to 10,000 m³ optionally in a continuous manner, and optionally so that the final volume of the culture medium is not more than three-fold of the volume of the culture medium before the addition of the acceptor feed and wherein optionally the pH of the lactose feed is set between 2.0 and 10.0 and wherein optionally, the temperature of the lactose feed is kept between 20° C. and 80° C.;
adding a lactose feed in a continuous manner to the culture medium over the course of 1 day, 2 days, 3 days, 4 days, 5 days by means of a feeding solution; and
adding a lactose feed in a continuous manner to the culture medium over the course of 1 day, 2 days, 3 days, 4 days, 5 days by means of a lactose feeding solution and wherein the concentration of the lactose feeding solution is 50 g/L and wherein optionally the pH of the lactose feeding solution is set between 2.0 and 10.0 and wherein optionally, the temperature of the lactose feeding solution is kept between 20° C. and 80° C.;
the method resulting in a sialylated oligosaccharide produced from the lactose with a concentration of at least 50 g/L in the final volume of the culture medium.

89. The method according to claim 88, wherein the lactose feed is accomplished by adding lactose from the beginning of the cultivation in a concentration of at least 5 mM.

90. The method according to claim 88, wherein the lactose feed is accomplished by adding lactose to the cultivation medium in a concentration, such, that throughout the production phase of the cultivation a lactose concentration of at least 5 mM is obtained.

91. The method according to claim 83, wherein the host cells are cultivated for at least about 60, 80, 100, or about 120 hours or in a continuous manner.

92. The method according to claim 83, wherein the cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract.

93. The method according to claim 83, wherein the cell uses at least one precursor for the synthesis of the sialylated di- and/or oligosaccharide.

94. The method according to claim 83, wherein the culture medium contains at least one compound selected from the group consisting of lactose, galactose, sialic acid, fucose, GlcNAc, GalNAc, lacto-N-biose (LNB), and N-acetyllactosamine (LacNAc).

95. The method according to claim 83, wherein a first phase of exponential cell growth is provided by adding a carbon-based substrate to the culture medium before the precursor is added to the culture medium in a second phase.

96. The method according to claim 83, wherein the cell produces at least one precursor for the synthesis of the sialylated di- and/or oligosaccharide.

97. The method according to claim 85, wherein the precursor for the synthesis of the sialylated di- and/or oligosaccharide is completely converted into the sialylated di- and/or oligosaccharide.

98. The method according to claim 83, wherein the sialylated di- and/or oligosaccharide is separated from the culture medium and/or the cell.

99. The method according to claim 83, wherein the separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis, affinity chromatography, ion exchange chromatography, hydrophobic interaction chromatography and/or gel filtration, ligand exchange chromatography, electrodialysis.

100. The method according to claim 83, wherein the method further comprises purification of the sialylated di- and/or oligosaccharide.

101. The method according to claim 100, wherein the purification comprises at least one of the following steps: using activated charcoal or carbon, using charcoal, nanofiltration, ultrafiltration, electrophoresis, enzymatic treatment or ion exchange, temperature adjustment, pH adjustment, pH adjustment with an alkaline or acidic solution, using alcohols, using aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.

102. A method for the fermentative production of a sialylated di- and/or oligosaccharide, wherein the improvement comprises:
utilizing a sialic acid transporter selected from SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59, or a functional homolog, variant or derivative of any one of the sialic acid transporters selected from SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59 having at least 80% overall sequence identity to the full-length sequence of any one of the polypeptide of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 47, 58 or 59, respectively, and wherein the functional homolog, variant or derivative has sialic acid transporter activity in the fermentative production of a sialylated di- and/or oligosaccharide.

103. A method of using the cell of claim 54 to produce a sialylated di- and/or oligosaccharide, the method comprising:
cultivating the cell.

104. The method according to claim 103, wherein a mixture of oligosaccharides comprising at least one sialylated oligosaccharide is produced.

105. The method according to claim 103, wherein a mixture of di- and oligosaccharides comprising at least one sialylated di- and/or oligosaccharide is produced.
